(19) United States
(12) Patent Application Publication
(10) Pub. No.: US 2026/0256855 A1
(43) Pub. Date: Sep. 3, 2026

(54) EXTRACELLULAR VESICLES FOR USE IN THERAPY

(71) Applicants: HUN-REN TÁMOGATOTT KUTATÓCSOPORTOK IRODÁJA, Budapest (HU); SEMMELWEIS EGYETEM, Budapest (HU)

(72) Inventors: Ádám VANNAY, Érd (HU); Beáta HRICZÓNÉ SZEBENI, Budapest (HU); Apor VERES-SZÉKELY, Budapest (HU); Domonkos PAP, Budapest (HU); Zoltán VARGA, Gárdony (HU)

(21) Appl. No.: 18/875,337

(22) PCT Filed: Jun. 14, 2023
(86) PCT No.: PCT/HU2023/050035

§ 371 Date: Dec. 16, 2024

(30) Foreign Application Priority Data
Jun. 14, 2022 (HU) P2200214

(51) Int. Cl. A61K 36/05; A61K 35/748; A61P 29/00

(52) U.S. Cl. CPC A61K 36/05; A61K 35/748; A61P 29/00

(57) ABSTRACT

The invention relates to algal extracellular vesicles (EVs) for use in therapy and in restoring or maintaining health of a subject. In particular the invention relates to uses of algal EVs in therapy, wherein the EVs themselves have favourable effects for the health of the subject to whom they are administered. In particular the algal EVs are for use in therapy in conditions or disorders associated with cell proliferation, and/or cell migration and/or non-physiological ECM production and deposition. The disorders also include in particular inflammation, neoplastic disorders, fibroproliferative diseases and the like. The invention also relates to methods of treatment of the subject as well as pharmaceutical and nutraceutical compositions. The invention also relates to cosmetic methods wherein algal EVs are applied to the affected skin.

### FIELD OF THE INVENTION

The invention relates to algal extracellular vesicles (EVs) for use in therapy and in restoring or maintaining health of a subject.

In particular the invention relates to uses of algal EVs in therapy, wherein the EVs themselves have favourable effects for the health of the subject to whom they are administered.

In particular the algal EVs are for use in therapy in conditions or disorders associated with cell proliferation, and/or cell migration and/or non-physiological ECM production and deposition. The disorders also include in particular inflammation, neoplastic disorders, fibroproliferative diseases and the like.

The invention also relates to methods of treatment of the subject as well as pharmaceutical and nutraceutical compositions. The invention also relates to cosmetic methods wherein algal EVs are applied to the affected skin.

### BACKGROUND OF THE INVENTION

The use of algae derived EVs as a drug delivery construct in cancer treatment is contemplated by Kuruvinashetti et al. (Algal Extracellular Vesicles for Therapeutic Applications. 2020 IEEE 20th International Conference on Nanotechnology (IEEE-NANO) July 29-31 2020, Virtual Conference), though without any experimental evidence or direct reference thereto.

CN110448696A describes a method for the preparation and use of marine algae (Dunaliella salina) derived EVs for use as a drug delivery construct. CN111543637A, CN111567798A and WO2021032794A1 also describe the use of (brown) algae derived EVs as a drug delivery construct. WO2011090731 describe microalgae producing a heterologous protein and EVs derived from such algae as the delivery construct for the heterologous protein. CN113209140 and CN113462645 describe the use of EVs derived from Phaeophyceae (multicellular brown algae) for activating NK cells by increasing e.g. the expression of TNFα. However, a closer look at the method described in these patent applications reveals that the first step of preparing the EVs is to obtain fucoidan from these seaweeds. In other embodiments, powdered fucoidan is used to prepare the EVs. Fucoidan is a sulfated polysaccharide well studied for its biological activities and possible therapeutic applications (Luthuli et al. Therapeutic Effects of Fucoidan: A Review on Recent Studies. Mar Drugs. 2019; 17 (9): 487. Published 2019 Aug. 21. doi: 10.3390/md17090487). It appears therefore that neither CN113209140, nor CN113462645 provide evidence of actual isolation of EVs or teach specifically the use of algae derived EVs as active agents in therapy. If such use were taught in either CN113209140 or CN113462645, that would have been a use as a delivery construct of fucoidan, similar to that described in the applications mentioned above. It is to be noted that fucoidan is typical of brown algae and not to monocellular or green algae.

Algae are widely used in food and food supplement and several health benefits are attributed to them. However, the numerous compounds making up algal cells may have different, even opposite effects or effects of certain compounds (or extracts) may be masked by other compounds (or extracts).

The inventors have found that well-defined algal extracellular vesicles are useful for use in therapy.

### BRIEF DESCRIPTION OF THE INVENTION

1. The invention relates to algal extracellular vesicles (EVs) for use in therapy, preferably in therapy of a disorder. Preferably, the invention relates to algal EVs for use in therapy of a disorder in a subject. The subject is an animal. In particular the subject is vertebrate animal, e.g. a fish, an amphibian or a reptile, preferably a mammal or a bird, highly preferably a mammal, in particular a human subject.

Algae as used herein are photosynthetic prokaryotic or eukaryotic organisms, that do not belong to Embryophyta (land plants).

Algae as used herein are preferably organisms that live in water (freshwater or seawater).

Prokaryotic algae as used herein are preferably prokaryotic photoautotroph organisms.

In an other embodiment prokaryotic algae used herein are heterotroph organisms.

Prokaryotic algae as used herein are organisms that preferably belong to Cyanobacteriota, preferably to Cyanophyceae.

Eucaryotic algae as used herein are preferably eukaryotic photoautotroph organisms, that do not belong to Embryophyta.

Eucaryotic algae as used herein are preferably photosythetic and/or heterotrophs organisms that do not have the ground tissues characteristic of plants (parenchyma, collenchyma and sclerenchyma), nor specialized reproductive organs.

Eucaryotic algae as used herein are organisms that are preferably selected from the following groups: Chlorophyta, Streptophyta, Rhodophyta (red alga), Ochrophyta (brown alga) and Cyanobacteriota phylum.

Preferably algae used herein are as defined in the detailed description.

In a highly preferred embodiment the algal EV is derived from the following selected species Chlorella sorokiniana, Parachlorella kessleri, Chlorella vulgaris, Chlorella pyrenoidosa (Auxenochlorella pyrenoidosa), Chlamydomonas reinhardtii, Hormidiospora verrucosa, Haematococcus lacustris, Tetraselmis chui, Zygnema peliosporum, Klebsormidium nitens, Spirogyra sp., Vischeria polyphem, Ascophyllum nodosum, Palmaria palmata, Microcystis aeruginosa, Nostoc linckia, Spirulina platensis (Arthrospira platensis), Spirulina maxima (Limnospira maxima), Arthronema africanum, Synechococcus sp., Aphanizomenon flos-aquae or other species of the same genus.

Algal EVs according to the present invention are preferably derived from eucaryotic algae.

Algal EVs according to the present invention are preferably derived from procaryotic algae.

Algal EVs according to the present invention are preferably derived from micro algae existing individually or in chains or groups.

Algal EVs according to the present invention are preferably derived from macro algae existing individually or in chains or groups.

Algal EVs according to the present invention are derived from freshwater algae.

Algae according to the present invention are derived from saltwater and/or marine algae.

Preferably the Algal EVs according to the present invention are derived from eucaryotic freshwater algae, in a preferred variant being different from brown algae and/or different from red algae.

In a particularly preferred embodiment the algae are green algae, preferably freshwater green algae.

In a particularly preferred embodiment the algae are green algae, preferably saltwater green algae.

In a particularly preferred embodiment the algae are food-quality algae, preferably dried algae.

In a particularly preferred embodiment the EVs are provided in the form of a medical device, pharmaceutical, cosmetic or nutraceutical composition comprising one or more pharmaceutically or nutraceutically acceptable excipients.

In a particularly preferred embodiment the EVs are provided in the form of a pharmaceutical or nutraceutical composition comprising one or more pharmaceutically or nutraceutically acceptable excipients.

In a preferred embodiment EV types from multiple sources are combined.

In a preferred embodiment EVs used as active agents are combined with medicaments or other active agents against the same disease. 2. Preferably the algal EVs are for use in the therapy of a disorder associated with cell proliferation, in particular excessive cell proliferation, and/or cell migration, in particular pathological cell migration and/or ECM-formation impairment, in particular impairment of regulation of ECM-formation, more particularly non-physiological (abnormal, non-healthy) ECM production and/or physiological ECM production, or deposition, in particular to regulate ECM production and/or deposition. Cell proliferation, migration and ECM production and/or deposition are processes which are often related and in particular are inter-related in a number of pathological processes (see FIG. 108). Preferably the disorder is manifested due to cell proliferation, and/or migration and/or ECM formation impairment, e.g. ECM production and/or deposition, in particular excessive cell proliferation, and/or pathological cell migration and/or non-physiological ECM production/deposition. In a particular embodiment algal EVs are for use in the therapy of a fibrotic, in particular a fibroploriferative disorder preferably as defined in paragraph 4. In particular the fibrotic, in particular a fibroploriferative disorder is related to cell migration and/or cell proliferation and/or ECM-formation impairment.

In a particular embodiment algal EVs are for use in the therapy of a neoplastic, preferably tumorogenic disorder preferably as defined in paragraph 5. In particular the neoplastic disorder is related to cell migration and/or cell proliferation and/or ECM-formation impairment.

In a particular embodiment algal EVs are for use in the therapy of an immunological disorder preferably as defined in paragraph 6. In particular the immunological disorder is related to cell migration and/or cell proliferation and/or ECM-formation impairment.3. The invention preferably relates to algal EVs for use in therapy according to any of paragraphs 1 or 2 wherein the disorder is a proliferation/migration disorder.

A proliferation/migration disorder is a disorder wherein both cell proliferation and migration play an important part in the condition (in particular a non-healthy condition) to develop (e.g. in disease etiology and/or manifestation) together with non-physiological ECM production or deposition in certain stage(s) of the condition.

Typically such proliferation/migration disorders are progressive fibrosis or tissue scarring, neoplastic conditions or cancers, in particular solid tumours, inflammation and conditions and stages leading or potentially leading to their manifestation.

In a particular embodiment the proliferation/migration disorders are progressive fibrosis or tissue scarring, neoplastic conditions or cancers.

In a particular embodiment the algal EVs for use according to the invention has antiproliferative effect.

4. The invention relates to algal EVs for use in therapy according to any of the above paragraphs, e.g. according to paragraph 3 wherein the disorder e.g. the proliferation/migration disorder is fibrosis comprising progressive fibrosis or is a fibroproliferative disease, in particular a fibroproliferative disease involving progressive fibrosis. In a particular embodiment involving fibrosis of the skin, or fibrosis in the gastrointestinal organs, in particular the intestine, or of the peritoneum or of abdominal organs, or of the kidney or of the lung or of the heart.

In a particular embodiment fibrosis is the fibrosis of the lung. In a particular embodiment fibrosis is the fibrosis of the peritoneum.

The fibrosis (e.g. progressive) as defined herein may occur actually at any part of the subject's body where fibrosis, in particular progressive fibrosis may occur.

In an embodiment the EVs inhibit ECM production.

In a further embodiment fibrosis is considered herein as a dynamic process of ECM production or degradation. An abnormal process (like injury on the one hand and progressive fibrosis on the other) shifts the equilibrium of this dynamic process to the extreme or removes from equilibrium. In a variant the ECM production/degradation becomes irregulated. In a preferred embodiment the EVs or the compositions of the invention improve the regulation of ECM production, preferably to maintain the dynamic nature thereof or maintain the normal equilibrium.

5. The algal EVs for use in therapy according to any of the above paragraphs, e.g. according to paragraph 3 wherein the disorder e.g. the proliferation/migration disorder is a neoplastic disease, preferably cancer, preferably tumourgenesis, in particular a solid tumour.

6. The algal EVs for use in therapy according to paragraph 3 wherein the proliferation/migration disorder is inflammation, in particular acute or chronic inflammation, and/or may be categorized as migratory/proliferative inflammation.

Preferably the disorder is an inflammatory disorder, in particular an autoimmune disorder. In an embodiment the inflammation is present in a chronic disorder. In an embodiment the inflammation is present in an autoimmune disorder. Preferably the algal EVs are for use as an anti-inflammatory agent.

Inflammation can be induced by a number of agents or causes and, as to its origin, may be of toxicological origin, e.g. induced by a chemical agent, mechanical origin like injury, oxidative stress origin, may be of biological origin like in the case of autoimmune diseases, or infection etc.

In an embodiment the EVs inhibit migration in inflammation, or immune cell migration. In an embodiment the EVs inhibit immune cell recruitment.

In an embodiment the EVs inhibit proliferation in inflammation or proliferative inflammation.

In further preferred embodiment inflammation is selected from:
fibrosis associated inflammation, in particular progressive fibrosis associated inflammation,
cancer associated inflammation,
inflammation related to tissue degeneration, e.g. arthritic inflammation
autoimmune disease associated inflammation.

7. In an embodiment the disorder according to paragraphs of any of 1 to 6 is an advanced glycation end product (AGE)-associated disease as defined herein.

Advanced glycation end products (AGEs) are proteins, lipids or nucleic acids that become glycated as a result of exposure to sugars and are involved in the development or worsening, of oxidative stress and inflammation related diseases such as metabolic syndrome, diabetes, inflammatory bowel disease, atherosclerosis, chronic kidney disease, Alzheimer's disease. For example AGE associated diseases are selected from the group consisting of these diseases and associated conditions. As an example, the AGE-associated disease is diabetic ulcus, which is characterized by disintegrated ECM, which may also be treated with EV-s, which can also increase ECM production/deposition.

Preferably the EVs are used as an anti-AGE agent. Preferably the EVs are used as inhibitor of AGE formation.

Preferably the EVs are used as inhibitor of the activation of AGE receptors.

8. The algal EVs for use in therapy of a disorder preferably in the vertebrate subject according to any of paragraphs 1 to 6 wherein the disorder is a disorder of the
skin, (preferred)
gastrointestinal organs, in particular the intestine, (preferred) (colon)
abdominal organs (liver),
cardiovascular, in particular the heart
peritoneum,
urinary system in particular the kidney,
respiratory system in particular the lung.

In a particular embodiment the EVs penetrate into the cells of the animal, in particular vertebrate (e.g. as defined in paragraph 1) subject, preferably cells of any of the organs as defined herein.

wherein preferably proliferation and migration of fibroblasts contribute to the manifestation of the disease.

EVs can pass (or get through) barriers of the body, like blood-brain barrier, the blood-eye-barrier, epithelial layer, endothelial layers etc. Thus fibrosis, inflammation or cancer may be present in the brain or in the eye etc. 9. The algal EVs for use in therapy according to any one of paragraphs 1 to 8 wherein the EVs are for use in inhibiting the activity of a signalling pathway selected from the group consisting of
PDE
PDGF receptor alpha and/or PDGF receptor beta signaling pathways, preferably PDGF-induced signaling, preferably PDGF-BB induced signalling pathway, wherein if PDGF-BB induced signalling pathway is mentioned herein it can be understood this broader way,
TGFβ, preferably TGFbeta-1 induced signalling pathways, wherein if TGFbeta-1 or TGFbeta induced signalling pathway is mentioned herein it can be understood this broader way,
EGF induced signalling pathway.

In a particular embodiment
the EVs are for use in inhibiting cell migration, preferably EGF induced cell migration,
the EVs are for use in inhibiting cell proliferation, preferably PDGF-BB induced cell proliferation, and/or
the EVs are for use in inhibiting ECM production, preferably TGFβ induced ECM production/deposition.
the EVs are for use in inhibiting a condition selected from cell migration, cell proliferation and/or inflammation and/or ECM production, preferably a growth factor mix induced or in particular PDE induced condition.

In particular embodiments the EVs are for use in inhibiting PDE, EGF, PDGF-BB and/or TGFB induced cell migration.

In particular embodiments the EVs are for use in inhibiting PDE, EGF, PDGF-BB and/or TGFB induced cell proliferation.

In particular embodiments the EVs are for use in inhibiting PDE, EGF, PDGF-BB and/or TGFB induced inflammation.

In particular embodiments the EVs are for use in inhibiting PDE, EGF, PDGF-BB and/or TGFβ induced ECM production/deposition.

In particular embodiments the EVs are for use in inhibiting cell migration, cell proliferation, inflammation and/or ECM production induced by an agent comprising multiple growth factors or a mixture of growth factors, such as a mixture of EGF, PDGF-BB and/or TGFB, like PDE.

In particular embodiments:

The algal EVs for use in therapy of disorders caused by cell migration wherein cell migration is induced by PDE EGF signalling pathway activation. Preferably, administration of algal EVs reduce cell migration.

The algal EVs for use in therapy of disorders caused by cell proliferation wherein cell proliferation is induced by PDE PDGF-BB signalling pathway activation is activated to induce cell proliferation and wherein administration of algal EVs reduce cell proliferation.

The algal EVs for use in therapy of disorders caused by ECM-production wherein ECM-production is induced by PDE TGFB signalling pathway activation. Preferably, administration of algal EVs reduce/modify ECM production. Preferably, administration of algal EVs regulate ECM production. Preferably, administration of algal EVs increase physiological ECM production and reduce non-physiological ECM production.

Signalling pathway activation involves activation of a part of said pathway (partial activation) provided that said activation results in activation of receptors of PDGF-BB and/or TGF-β and/or EGF (i.e. as used herein PDGF-BB and/or TGF-β and/or EGF receptors).

The algal EVs for use in therapy according to any one of paragraphs 1 to 9 wherein said EVs are for use in inhibiting the activity of a signalling pathway selected from the group consisting of
a signalling pathway induced by multiple growth factors or a mixture of growth factors like PDE
PDGF-BB induced signalling pathway,
TGFβ induced signalling pathways,
EGF induced signalling pathway, wherein in said disorder one or more of the PDGF-BB and/or TGF-β and/or EGF receptors are activated.

Alternative wording, EVs are for use in inhibiting activity of said pathways in particular activation of any or more of said receptors.

The invention relates to the method as defined herein wherein the level or phosphorylation or localization or activation of one or more the following factors and or signalling molecule (preferably at least 2, 3, 5, 7, 10 or 15 is/are altered, i.e. different from a normal range defined for a population of healthy people, e.g. a population having a common trait relevant under the conditions:

| | |
|---|---|
| AKT1 | AKT serine/threonine kinase 1 |
| EGF | epidermal growth factor - level |
| FOS | Fos proto-oncogene, AP-1 transcription factor subunit |
| GRB2 | growth factor receptor bound protein 2 |
| HRAS | HRas proto-oncogene, GTPase |
| JAK1 | Janus kinase 1 |
| JUN | Jun proto-oncogene, AP-1 transcription factor subunit |
| MAP2K1 | mitogen-activated protein kinase kinase 1 - phosphorilation |
| MAP2K2 | mitogen-activated protein kinase kinase 2 |
| MAPK1 | mitogen-activated protein kinase 1 |
| MAPK3 | mitogen-activated protein kinase 3 |
| MTOR | mechanistic target of rapamycin kinase |
| STAT1 | signal transducer and activator of transcription 1 |
| STAT3 | signal transducer and activator of transcription 3 |

In alternative embodiments the level of the factors are listed in any one of tables 4 to 6.

10. The invention relates to the algal EVs for use in therapy according to any one of paragraphs 1 to 9 wherein the disorder is selected from the groups consisting of
a fibroproliferative disorder,
a neoplastic disorder,
an inflammatory disorder.

11. The invention relates to the algal EVs for use in therapy according to any one of paragraphs 1 to 10 wherein the EVs are derived from green, brown red or Cyanobacteriota algae, preferably from green algae (e.g. Chlorophyta or Streptophyta), brown algae (e.g. Ochrophyta), red algae (e.g. Rhodophyta)—blue algae (e.g. Cyanobacteriota).

The algae are preferably microalga or macroalga.

In a particular embodiment the algae are for use in therapy according to any one of the preceding paragraphs, wherein the EVs are derived from Chlorophyta, Rhodophyta, Ochrophyta and Streptophyta phylum preferably from Chlorella sorokiniana, Parachlorella kessleri, Chlorella vulgaris, Chlorella pyrenoidosa (Auxenochlorella pyrenoidosa), Chlamydomonas reinhardtii, Hormidiospora verrucosa, Haematococcus lacustris, Tetraselmis chui, Zygnema peliosporum, Klebsormidium nitens, Spirogyra sp., Vischeria polyphem, Ascophyllum nodosum, Palmaria palmata

In a particular embodiment the algae are for use in therapy according to any one of the preceding paragraphs, wherein the EVs are derived from Cyanobacteriota phylum, preferably from Microcystis aeruginosa, Nostoc linckia, Spirulina platensis (Arthrospira platensis), Spirulina maxima (Limnospira maxima), Arthronema africanum, Synechococcus sp., Aphanizomenon flos-aquae.

In particular the EVs are preferably derived from green algae. The EVs are preferably derived from sweet and saltwater phylums including Chlorophyta, Streptophyta, Rhodophyta, Ochrophyta and Cyanobacteriota. In a highly preferred embodiment the alga is selected from Chlorella sorokiniana, Parachlorella kessleri, Chlorella vulgaris, Chlorella pyrenoidosa (Auxenochlorella pyrenoidosa), Chlamydomonas reinhardtii, Hormidiospora verrucosa, Haematococcus lacustris, Tetraselmis chui, Zygnema peliosporum, Klebsormidium nitens, Spirogyra sp., Vischeria polyphem, Ascophyllum nodosum, Palmaria palmata, Microcystis aeruginosa, Nostoc linckia, Spirulina platensis (Arthrospira platensis), Spirulina maxima (Limnospira maxima), Arthronema africanum, Synechococcus sp., Aphanizomenon flos-aquae.

In a preferred embodiment the alga is selected from as given in the Definitions.

In a preferred embodiment the alga is selected from as given in the DETAILED DESCRIPTION, in particular in chapter Algae useful in the invention.

12. The invention relates to the algal EVs for use in therapy according to any one of paragraphs 1 to 11 wherein said algal EVs are prepared by a process comprising the steps of
providing isolated alga cells; in particular an alga cell culture, preferably wherein alga cells are propagated in a medium, or dried, preferably rehydrated freeze-dried or spray-dried alga cells,
obtaining EVs from said alga cells
preferably formulating said EVs into an EV preparation.

Isolation is understood herein as a process wherein the natural environment of the EVs are changed artificially, i.e. by human action which is a technical step. Preferably the EV population is also changed by selecting EVs according to their size and removing cellular material, and/or debris, and or other bioactive molecules.

The invention preferably relates to the method of the preparation of the algal EVs or compositions comprising said EVs.

The EVs and/or EV compositions (or preparations) prepared by the technology according to the invention can be used in any one of the indications (or therapies or methods of treatment or in the treatment of disorders) as specified above, e.g. in any one or a combination of paragraphs 1, 2, 3, 4, 5, 6, 7, 8, 9, 10 and/or 11.

Preferably EVs from any one of algae as defined in any one of or both of paragraphs 1 and 11 can be used.

In a particular embodiment the algae from which the EV-s are isolated are dried, preferably rehydrated freeze-dried or spray-dried alga cells.

In a particular embodiment the EVs are freeze-dried or spray-dried.

13. In particular the invention relates to the algal EVs for use in therapy according to paragraph 12, wherein said algal EVs are obtained
from supernatant of a culture of the alga cells, by isolating EVs from the culture supernatant,
from dried alga cells by rehydration of said alga cells with a medium, removing, in particular pelleting the cells and cell debris and isolating EVs from the supernatant medium,
from suspension of alga cells, in particular of cultured alga cells or dried and rehydrated alga cells, in particular rehydrated pelleted alga cells, preferably applying by a mechanical or physical effect.

14. In particular the invention relates to the algal EVs for use in therapy according to paragraph 12 or 13 wherein said algal EVs are obtained by a method comprising the steps of
removing alga cells and/or cellular components comprising debris thereof by a technique selected from the group consisting of ultracentrifugation, centrifugation, filtration, tangential flow filtration (TFF), size exclusion chromatography (SEC), dialysis of the supernatant of algal cells, wherein preferably the cellular supernatant is also removed.

15. In particular the invention relates to the algal EVs for use in therapy according to any one of paragraphs 12 to 14 wherein obtaining the EVs from the alga cells comprises filtration, preferably ultrafiltration (UF).

16. In particular the invention relates to the algal EVs for use in therapy according to any one of paragraphs 12 to 15 wherein obtaining the EVs from the alga cells comprises size exclusion chromatography (SEC), preferably SEC on a SEC chromatography matrix, preferably having a pore size of 10 to 1000 nm, preferably 10 to 500 nm, or 35 to 500 nm, or 35 to 350 nm, in particular in particular 70-1000 nm, or 70 to 500 nm. Highly preferably the medium pore size is 60-80 nm including of about 70 nm.

Optionally the method comprises preparing the SEC column, optionally by liposomes. Preferably the size (average diameter) of the liposomes is as defined as the pore size for the EVs above.

In a further preferred embodiment the SEC column is prepared by EVs. Preferably the EVs are identical to or similar to those to be prepared on the SEC column.

17. In particular the invention relates to the algal EVs for use in therapy according to any one of paragraphs 12 to 16 wherein
separating the EVs from the alga cells comprises concentration of the EVs. Preferably concentration is carried out by filtration, eg. UF. In particular with a cut-off allowing proteins and nucleic acids to flow through into the filtrated. The cut-off is e.g. 20 to 500 kDa, 20 to 400 kDa, 20 to 300 kDa, or 50 to 400 kDa, 50 to 300 kDa. Alternatively, the cut-off is e.g. 20 to 200 kDa, preferably 50 to 200 kDa or 50 to 150 kDa, highly preferably about 100 kDa. In an embodiment multiple membranes with different cut-offs are applied.

In a particular embodiment cell are removed by a cell filter, e.g. a bacterial filter e.g. of about 0.2 micrometer pore size.

18. In particular the invention relates to the algal EVs for use in therapy according to any one of paragraphs 1 to 17 wherein the size intensity distribution of said algal EVs is characterized by an intensity distribution in the order of 10 or 102 to 103 nm, or 10 to 102 nm, in particular 1.8×10² to 1.8×10³ nm intensity distribution.

Highly preferably the majority of the size (or diameter) EVs is 70 to 500 nm. In various embodiments the diameter of the EVs is 10 to 1000 nm, preferably 10 to 500 nm, or 35 to 500 nm, or 35 to 350 nm, in particular 70-1000 nm. Alternatively, at least 70%, 80%, 90% or at least 95% or at least 98% of the EVs have the diameter in the range given herein.

19. In particular the invention relates to the algal EVs for use in therapy according to any one of paragraphs 1 to 18 wherein
the EVs are used as an active agent for use in a condition as defined in any one or a combination of paragraphs 1, 2, 3, 4, 5, 6, 7, 8, 9, 10 and/or 11, whereas
the EVs comprise a further active agent for use in the treatment of the same disorder as defined in any of paragraphs 1 to 10; or
wherein in said therapy the EVs are administered together with a further active agent for use in the treatment of the same disorder as defined in any of paragraphs 1 to 10.

Preferably the EVs are defined in any one of the previous paragraphs, in particular in paragraphs 1 and/or 11 and or by methods as defined in any of the paragraphs 12 to 18.

Preferably the disorder as defined in any of paragraphs 1 to 10 or 11,
wherein said further active agent is an anti-fibrotic agent,
wherein said further active agent is an anti-neoplastic agent,
wherein said further active agent is an anti-inflammatory agent.

In a particular embodiment the EVs or a combination or a composition thereof comprises, as a further active agent, a compound selected from vitamins, trace elements, roborants, etc.

In a particular embodiment said algae are genetically modified algae to produce an protein or nucleic acid active agent.

20. The algal EVs for use in therapy according to any one of paragraphs 1 to 19 wherein in said therapy the EVs are administered
orally, preferably in the form of a composition for oral administration,
topically, preferably in the form of a composition for topical administration,
intraperitoneally, preferably in the form of a composition for intraperitoneal administration,
intranasally, preferably in the form of a composition for intranasal administration,
subcutan/intracutan preferably in the form of a composition for subcutan administration,
intramuscular, preferably in the form of a composition for intramuscular administration, or
intravenous, preferably in the form of a composition for intravenous administration.
In a particular embodiment administration is intratumoral.
In a particular embodiment administration is intraocular.

In a particular embodiment administration is oral administration.

In a particular embodiment administration is intraperitoneal administration.

In a particular embodiment administration is intravenous administration.

In a particular embodiment administration is intracutan administration.

21. The invention also relates to (in a further aspect) a composition for use in the treatment of a disorder as defined in any of the previous paragraphs, in particular in paragraphs 1 to 10 said composition comprising algal EVs as an active agent and/or a biologically acceptable carrier for a mammalian subject.

Preferably the EVs are defined in any one of paragraphs 1 and/or 11.

22. The invention also relates to the composition according to paragraph 21 comprising algal EVs as defined in any of paragraphs 11 to 20.

In a particular embodiment the number of algal EVs in a dose unit of the composition is at least 10³ preferably at least 10⁵, or at least 10⁶ or at least 10⁷ or at least 10⁸ or at least 10⁹ or at least 10¹⁰. In an embodiment the number of algal EVs in a dose unit of the composition is at most 10²⁰ preferably at most 10¹⁵, or at most 10¹² or at most 10¹¹. In particular the number of algal EVs in a dose unit of the composition is at least or at least 3×10⁶ or at least 3×10⁷ or at least 3×10⁸ or at least 3×10⁹. Preferably, the number of algal EVs in the composition is at least 3×10⁷ or at least 3×10⁸ or at least 3×1×10⁹ or at least 3×10¹⁰.

In an embodiment the above data relate to concentration and 1 ml of dose unit.

In a particular example a dose for the treatment of an animal is at least 3×10⁷ EVs or 3×10⁸ or 3×10⁹ EVs per treatment, e.g. in a mice as a model animal. In mice the proposed dose corresponds to about at least 1.5×10⁹ EVs or at least 1.5×10¹⁰ EVs per kilogram body weight (kgbw). In larger animals like in humans the dose may be smaller per kgbw, e.g. 1.5×10⁸ EVs or at least 1.5×10⁹ EVs per kgbw.

In a particular embodiment the dose range in a dose unit of the composition in case of mammals e.g. in humans is at least 10⁵ and at most 10⁸ EVs per dose or at least 10⁶ and at most 10⁹ EVs per dose or at least 10⁷ and at most 10¹⁰ EVs per dose or at least 10⁸ and at most 10¹¹ EVs per dose. In an embodiment this is a daily dose.

23. The invention also relates to the composition according to any of paragraphs 21 or 22 said composition being a pharmaceutical composition said composition comprising a pharmaceutically acceptable carrier.

24. The invention also relates to the composition according to any of paragraphs 21 to 23, said composition being formulated
for oral administration,
for topical administration,
for intraperitoneal administration,
for intranasal administration,
for subcutan administration,
for intramuscular administration, or
for intravenous administration.
for intratumoral administration.
for intraocular administration;
in a particular embodiment
   for oral administration and/or
   for intraperitoneal administration and/or
   for intravenous administration and/or
   for topical administration and/or
   for intracutan administration,
   for administration as defined in paragraph 20.

25. The invention also relates to the composition according to any of paragraphs 21 to 24, said composition also comprising a further active agent for use in the treatment of a disorder as defined in any of paragraphs 1 to 10. Preferably the EVs are defined in any one of the previous paragraphs, in particular in any one of paragraphs 1 and/or 11 or by a process according to any of claims 12 to 18.

Preferably the disorder as defined in any of paragraphs 1 to 10 or 11,
wherein said further active agent is an anti-fibrotic agent more preferably nintedanib;
wherein said further active agent is an anti-neoplastic agent more preferably axitinib or ponatinib;
wherein said further active agent is an anti-inflammatory agent; and/or
wherein said further active agent is an anti-AGE agent.

26. The invention also relates to the composition according to any of paragraphs 21 or 22 said composition being a nutraceutical composition, said composition comprising a nutraceutically acceptable carrier,
preferably selected from the group consisting of
food (dietary) supplement, a fortified food, a medicinal food, a functional food, a dietary formula, or a composition with a health paragraph.

Thus, nutraceutical is defined herein to include any composition including food which has a health-related purpose or use and/or is offered for such purpose or use.

27. The invention also relates to the composition according to any of paragraphs 21 to 26 said composition being a dermatological cosmetic composition said composition comprising a dermatologically tolerable excipient.

28. The invention also relates to the composition according to paragraph 27 wherein said composition is a cosmetic composition said composition comprising a dermatologically tolerable excipient.

29. The invention also relates to the composition according to any of paragraphs 27 to 28, said composition further comprising an agent against ECM deposition, preferably non-physiological ECM production or deposition.

30. The invention also relates to the composition according to any of paragraphs 21 to 29, said composition being formulated for storage, preferably said carrier comprising a medium for storage of the algal EVs.

Preferably the medium comprises stabilizers.

Preferably the medium comprises a substance to preserve EV integrity, e.g. a salt or sugar.

Preferably the EVs are concentrated.

Optionally the EVs are lyophilized or freeze-dried or spray-dried, preferably spray-dried.

31. The invention also relates to uses of EVs as defined herein in the manufacture of a composition for use in therapy, preferably in therapy of a disorder, preferably for use in therapy of a disorder in a subject, or in a method of treatment, each as defined herein. In particular, the disorder is any disorder as defined in any of the previous paragraphs, in particular in paragraphs 1 to 10 or 11. In particular, the composition is as defined in any of paragraphs 21 to 30, in particular a pharmaceutical composition or a nutraceutical composition as defined herein. In particular the algal EVs are as defined in any of the previous paragraphs, in particular in any one of paragraphs 1 and/or 11 or by a process according to any of paragraphs 12 to 18.

32. In a further aspect the invention relates to a method for treatment of a mammalian subject having a disorder as defined in any of paragraphs 1 to 11, said method comprising administration of said algal EVs to a mammalian subject.

33. The invention also relates to the method for treatment of the mammalian subject according to paragraph 32 said method comprising
oral administration of a composition of any of paragraphs 21 to 26 to said mammalian subject,
topical administration of a composition of any of paragraphs 27 to 29 to said mammalian subject.
intraperitoneal administration of a composition of any of paragraphs 21 to 23 to said mammalian subject.
intranasal administration of a composition of any of paragraphs 21 to 23 to said mammalian subject.
intramuscular administration of a composition of any of paragraphs 21 to 23 to said mammalian subject.
intravenous administration of a composition of any of paragraphs 21 to 23 to said mammalian subject.

34. The invention also relates to the method for treatment of the mammalian subject according to paragraph 32 or 33 said method comprising a regular administration of said composition, preferably administration in every month or week or two days, or preferably a daily administration.

35. The invention also relates to the method for treatment of the mammalian subject according to paragraph 32 or 33 said method comprising
an administration lasting for one week or 2 weeks, preferably at least one months or 2 months of said composition.

36. In a further aspect the invention relates to a cosmetic method for treatment of a mammalian to regulate/inhibit/induce extracellular matrix (ECM) production said method comprising administration of a cosmetic composition comprising algal EVs as an active agent.

In a particular embodiment ECM production/deposition is induced, e.g. when ulcer is treated.

In a particular embodiment ECM production/deposition is inhibited, e.g. when keloid is treated.

The cosmetic composition is a composition as defined herein comprising a dermatologically tolerable or useful excipient.

Preferably said subject is in need of inhibition of skin tissue scarring or extracellular matrix (ECM) production.

### Definitions

“Progressive fibrosis” in short is characterized by a process when ECM remodeling is shifted towards accumulation of ECM producing cells, like fibroblasts or myofibroblasts, and/or towards non-physiological, e.g. excessive deposition of ECM components leading to impairment or destruction of tissue architecture and/or to gradual decline of organ function, which is a kind of non-physiological ECM production/deposition.

Progressive fibrosis may lead to the formation of permanent scar tissue, may cause tissue or organ failure and might lead to death. In “progressive fibrosis” ECM components and ECM producing cells, in particular fibrillar ECM components like type I and III collagen and fibronectin, as well as the cells producing them continue to accumulate even beyond the homeostatic/regenerative phase of ECM remodeling.

The process in which an excessive amount of ECM replaces normal parenchyma or the ECM which is typical to the tissue affected by progressive fibrosis may also be considered “progressive fibrosis”. This process is characterized by overproliferation of ECM producing cells, e.g. fibroblasts, and excessive, unregulated or dysregulated deposition of ECM components and/or abnormal repair processes in different tissues upon injury.

Collagen accumulation can be measured by Sirius Red assay . . . .

“Fibroproliferative disorder” is a disorder which is characterized by inter alia the presence of progressive fibrosis, in particular wherein at least partially ECM remodeling is shifted towards accumulation of ECM producing cells, like fibroblasts, and/or towards excessive deposition of ECM components leading to impairment or destruction of tissue architecture and/or to gradual decline of organ function.

A “neoplasm” is a type of abnormal and excessive growth of tissue. (The process that occurs to form or produce a neoplasm is called “neoplasia”.) The growth of a neoplasm is uncoordinated with that of the normal surrounding tissue, and persists in growing abnormally, even if the original trigger is removed. This abnormal growth usually forms a mass, when it may be called a tumour. Tumour cells often metastasize to various organs.

“Inflammation” as defined herein as a process which is a biological response of an animal body, preferably a vertebrate or a mammalian body, in particular the immune system thereof, to a stimulus or stimuli recognized as harmful by the body in particular the immune system, typically involving injured or impaired cell(s), wherein inflammatory cells (macrophages, dendritic cells, granulocytes, lymphocytes, fibroblasts, endothelial and epithelial cells), and mediators, in particular cytokines, chemoattractants, complement, growth and angiogenic factors are produced in a level higher than in the same body without inflammation.

“Deposition of ECM” is understood herein as a process leading to an increase in the amount of ECM components in a space among/between (i.e. outside) the cells of a tissue.

A “physiological” or “regulated” or “normally regulated” deposition of ECM occurs when deposition of ECM components serves to restore tissue architecture and/or tissue function itself. The physiological deposition of ECM components is regulated or maintained under control to avoid non-physiological ECM deposition or production. Preferably, in in vivo setting the regulatory processes of the surrounding healthy tissue in question counter-acting deposition are capable of reversing, or at least arresting such deposition.

A “non-physiological” or “dysregulated” deposition of ECM occurs when deposition of ECM components leads to impairment, i.e. destruction of tissue architecture and/or tissue function itself. The unregulated or abnormally regulated deposition of ECM components is a particular hallmark of non-physiological ECM deposition or production. Preferably, deposition of ECM components is considered as “non-physiological” (in case of fibrosis excessive) when there are no signs that regulatory processes of the surrounding healthy tissue in question counter-acting deposition are capable of reversing, or at least arresting such deposition.

A “subject” as used herein is an individual of an animal species, preferably a vertebrate, more preferably a mammalian or avian species, in particular a mammalian species, highly preferably the individual is a primate, a hominid or a human.

The term “mammal’ is known in the art and relates to an animal species of which the female feeds her young on milk from her own body, and exemplary mammals include humans, primates, livestock animals (including bovines, porcines, goats, sheep, horses etc.), companion animals (e.g., canines, felines, etc.) and rodents (e.g., mice and rats) all or any of which is contemplated herein.

A “patient” is a subject who is or intended to be under medical or veterinarian observation, supervision, diagnosis or treatment.

A “treatment” refers to any process, action, application, therapy, or the like, wherein the subject or patient is under aid, in particular medical or veterinarian aid with the object of improving the subjects's or patient's condition, either directly or indirectly. Improving the subjects's condition may include improving an aesthetic condition (cosmetic treatment) and/or may include, in particular, restoring or maintaining normal function of an organ or tissue, preferably at least partly restoring or maintaining health (medical or veterinarian treatment). Treatment typically refers to the administration of an effective amount of a compound or composition described herein. Treatment may relate to or include medical or veterinarian treatment and cosmetic treatment, in particular medical or veterinarian treatment.

“Preventing” or “prevention” of the development of a disease or condition refers to at least the reduction of likelihood of the risk of or susceptibility to acquiring a disease or disorder, or preferably causing at least one of the clinical symptoms of the disease or disorder not to develop in a patient that may be exposed to or predisposed to the disease but does not yet experience or display symptoms of the disease.

The terms “effective amount” or “therapeutically effective amount” are intended to qualify the amount of a therapeutic agent required to relieve to some extent one or more of the symptoms of a condition, disease or disorder, including but not limited to: 1) reducing the number of fibroblasts or myofibroblasts; 2) reducing the synthesis of the ECM components, and/or increasing the degradation of the ECM component; 3) reducing the size of the fibrous tissue; 4) improving to at least some extent the physiological function of the tissue due to any of 1) to 3); 5) reducing the size of a tumour tissue; 6) inhibits the formation of tumour cell metastases; 7) inhibits immune cell proliferation or activation, including production of cytokines, growth factors or antibodies.

The compounds of the invention have pharmaceutical (medicinal), nutritional, and cosmetic uses as well.

A “composition” relates to a composition of matter for use in the human or animal body, said composition comprising an active agent (in particular alga EVs) and one or more additional substance useful as carrier. The term “carrier” refers to a diluent, adjuvant, filler, excipient, stabilizer, or vehicle with which the agent is formulated for administration.

A “pharmaceutical composition” relates to a composition for use in treatment of human or animal body to restore or maintain health, said composition comprising alga EVs as the active agent and one or more additional substance useful as carrier.

“Nutraceutical” refers to a foodstuff that provides health benefits in addition to its basic nutritional value. A nutraceutical has a physiological benefit or provide protection against physiological disorder or discomfort. A “neutraceutical composition” is a nutraceutical which comprises a composition of the invention and at least an additional substance, e.g. a neutraceutical carrier or a food component.

The term “dietary supplement” refers to a neutraceutical e.g. a neutraceutical composition intended to provide nutrients that may otherwise not be consumed in sufficient quantities.

“Functional food” is also a neutraceutical e.g. a neutraceutical composition and refers to any modified food or food ingredient that may provide a benefit or provide protection against physiological disorder or discomfort; beyond the traditional nutrients it contains.

A “health claim” defines a health benefit for a neutraceutical and is subject to regulatory approval (analogous to an indication in case of a medicament) in accordance with a national or equivalent law. A “health claim” is to be as food labels and in food marketing.

“Algae” as used herein are photosynthetic or heterotroph prokaryotic or eukaryotic organisms, that do not belong to Embryophyta (land plants).

Algae as used herein are preferably organisms that live in water (freshwater or seawater).

Prokaryotic algae as used herein are preferably prokaryotic photoautotroph organisms.

Prokaryotic algae as used herein are organisms that preferably belong to Cyanobacteria, preferably to Cyanophyceae.

Eucaryotic algae as used herein are preferably eukaryotic photoautotroph organisms, that do not belong to Embryophyta. Eucaryotic algae as used herein are preferably photosythetic organisms that do not have the ground tissues characteristic of plants (parenchyma, collenchyma and sclerenchyma), nor specialized reproductive organs.

Eucaryotic algae as used herein are organisms that preferably belong to the following groups: Chlorophyta, Streptophyta, Rhodophyta, Ochrophyta and Cyanobacteriota phylum

The singular forms “a”, “an” and “the”, or at least “a”, “an”, include plural reference unless the context clearly dictates otherwise.

The term “comprises” or “comprising” or “including” are to be construed here as having a non-exhaustive meaning and allow the addition or involvement of further features or method steps or components to anything which comprises the listed features or method steps or components. “Comprising” can be substituted by “including” if the practice of a given language variant so requires or can be limited to “consisting essentially of” if other members or components are not essential to reduce the invention to practice.

### BRIEF DESCRIPTION OF THE FIGURES

FIG. 1. Representative immunofluorescence staining of human primary peritoneal fibroblasts from patient A (phPFB/A) for α-smooth muscle actin (α-SMA, green) and cytokeratin (CK) 18 (red). Cell nuclei were counterstained with Hoechst 33342 (blue). Images were taken with 20× objective.

FIG. 2. Representative immunofluorescence staining of human primary peritoneal mesothelial cells (HPMC) for α-smooth muscle actin (α-SMA, green) and cytokeratin (CK) 18 (red). Cell nuclei were counterstained with Hoechst 33342 (blue). Images were taken with 20× objective.

FIG. 3. Representative immunofluorescence staining of human primary colon fibroblasts from patient A (phCFB/A) for α-smooth muscle actin (α-SMA, green) and cytokeratin (CK) 18 (red). Cell nuclei were counterstained with Hoechst 33342 (blue). Images were taken with 20× objective.

FIG. 4. Representative immunofluorescence staining of human primary colon fibroblasts from patient B (phCFB/B) for α-smooth muscle actin (α-SMA, green) and cytokeratin (CK) 18 (red). Cell nuclei were counterstained with Hoechst 33342 (blue). Images were taken with 20× objective.

FIG. 5. Representative immunofluorescence staining of human primary skin fibroblasts from patient A (phSFB/A) for α-smooth muscle actin (α-SMA, green) and cytokeratin (CK) 18 (red). Cell nuclei were counterstained with Hoechst 33342 (blue). Images were taken with 20× objective.

FIG. 6. Representative immunofluorescence staining of human primary skin fibroblasts from patient B (phSFB/B) for α-smooth muscle actin (α-SMA, green) and cytokeratin (CK) 18 (red). Cell nuclei were counterstained with Hoechst 33342 (blue). Images were taken with 20× objective.

FIG. 7. EVs of Chlorella sp. MACC-360 origin (red arrows) were visualized by transmission electron microscopy (TEM) after negative staining with uranyl acetate (−1). The average size/size distribution of EVs and their particle numbers was measured by dynamic light scattering (DLS) with the following data:

C360 fraction2_wo dil in PBS

| Date: | Jan. 13, 2022 | | Temperature: | 20° | C. | Data Filter: | Default |
|---|---|---|---|---|---|---|---|
| | 9:06:43 AM | | | | | | |
| Solvent: | PBS | | Solute: | — | | Laser: | 30% |
| Intensity: | 187,856 | counts/s | Intercept: | 0.675 | | Attenuator: | 1% |
| Z - Av. Diameter: | 684.30 | nm | Std. Deviation: | 731.56 | nm | MW model: | Globular Proteins |

| | | | | | |
|---|---|---|---|---|---|
| Polydispersity: | 106.91% | Pd. Index: | 1.143 | Remark: | — |

| | Mean Dh | Mode Dh | Std. Dev. | Polydisp. | Est. MW. | Intensity | Mass | Volume | Number |
|---|---|---|---|---|---|---|---|---|---|
| Peak # | (nm) | (nm) | (nm) | (%) | (kDa) | (%) | (%) | (%) | (%) |
| 1 | 185.60 | 189.33 | 99.32 | 53.52 | Out of | 25.02 | 98.66 | 99.55 | 100.00 |
| | | | | | Range | | | | |
| 2 | 1,535.64 | 1,653.45 | 874.66 | 56.96 | Out of | 74.98 | 1.34 | 0.45 | 0.00 |
| | | | | | Range | | | | |

and multiple resistive pulse sensing (MRPS).

| | |
|---|---|
| Run Details | |
| Cartridge class: | TS400 |
| Mold ID: | P12.4.2 |
| Cartridge Box Date: | 210303 |
| Sample Description: | 02 AC360 1 10xdil |
| Cal particle size (nm): | 1.0 |
| Cal particle concentration (/mL): | 1.00E+00 |
| Analysis | |
| Size range min (nm): | 65.0 |
| Size range max (nm): | 400.0 |
| Total concentration (/mL): | 1.05E+09 |
| Number of particles detected: | 1990 |
| Statistical error in concentration (%): | 2.5% |
| D10 (nm): | 68.4 |
| D50 (nm): | 81.8 |
| D90 (nm): | 128.7 |
| Data processing user name: | mrps |
| Measurement Parameters | |
| Measurement date: | 20210528 |
| Measurement user name: | mrps |
| Acquisition time (s): | 490.0 1.90E+00 |
| Measured volume (nL) | 1.000 |
| Diameter scaling factor | 1.000 |
| Concentration scaling factor | |

FIG. 8. Representative Western blots for CTGF, PDGF-B, and TGF-β content of peritoneal dialysis effluents (PDEs) derived from patients with different underlying kidney diseases.

FIG. 9. Penetration of EVs of Chlorella sp. MACC-360 origin to primary human peritoneal fibroblasts from patient A (phPFB/A). phPFB/A cells were treated with cell culture medium containing 3×10\hat{ }8 particles/ml of the DiI labelled EVs (red) for 24 hours. Images were taken with 20× objective.

FIG. 10. Penetration of EVs of Chlorella sp. MACC-360 origin to primary human peritoneal fibroblasts from patient B (phPFB/B). phPFB/B cells were treated with cell culture medium containing 3×10\hat{ }8 particles/ml of the DiI labelled EVs (red) for 24 hours. DiI labelled PBS (in the absence of EVs) was used as negative control. Cell nuclei were stained with Hoechst 33342 (blue). Images were taken with 20× objective.

FIG. 11. Penetration of EVs of Chlorella sp. MACC-360 origin to primary human peritoneal mesothelial cells (HPMC). HPMCs were treated with cell culture medium containing 3×10\hat{ }8 particles/ml of the DiI labelled EVs (red) for 24 hours. DiI labelled PBS (in the absence of EVs) was used as negative control. Cell nuclei were stained with Hoechst 33342 (blue). Images were taken with 20× objective.

FIG. 12. Penetration of EVs of Chlorella sp. MACC-360 origin to primary human colon fibroblasts from patient A (phCFB/A). phCFB/A cells were treated with cell culture medium containing 3×10\hat{ }8 particles/ml of the DiI labelled EVs (red) for 24 hours. Images were taken with 20× objective.

FIG. 13. Penetration of EVs of Chlorella sp. MACC-360 origin to primary human skin fibroblasts from patient A (phSFB/A). phSFB/A cells were treated with cell culture medium containing 3×10\hat{ }8 particles/ml of the DiI labelled EVs (red) for 24 hours. Images were taken with 20× objective.

FIG. 14. Effect of EV of Chlorella sp. MACC-360 origin on peritoneal dialysis effluent (PDE) induced proliferation of primary human peritoneal fibroblast from patient A (phPFB/A). phPFB/A cells were treated with PDE and with 3×10\hat{ }7 or 3×10\hat{ }8 particles/ml EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t test. ** p<0.01 3×10 vs. C (PBS), * p<0.05 3×10 vs. C (PBS), ##p<0.01 vs. C (PDF), $$3×10 vs. PDE.

FIG. 15. Effect of EV of Chlorella sp. MACC-360 origin on PDGF-B induced proliferation of primary human peritoneal fibroblast from patient A (phPFB/A). phPFB/A cells were treated with PDGF-B and with of 3×10\hat{ }7 or 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test. ** p<0.01 3×10 vs. C (PBS), * p<0.05 3×10 vs. C (PBS), ##p<0.01 vs. C (HCl), $$ p<0.01 3×10 vs.3×10 PDGF.

FIG. 16. Effect of EV of Chlorella sp. MACC-360 origin on PDE induced proliferation of primary human colon fibroblast from patient A (phCFB/A). phCFB/A cells were treated with peritoneal dialysis effluent (PDE) and with 3×10\hat{ }7 or 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. PDF, $$ p<0.01 3×10 vs.3×10 PDE.

FIG. 17. Effect of EV of Chlorella sp. MACC-360 origin on PDGF-B induced proliferation of primary human colon fibroblast from patient A (phCFB/A). phCFB/A cells were treated with PDGF-B and with 3×10\hat{ }7 or 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs.3×10 PDGF.

FIG. 18. Effect of EV of Chlorella sp. MACC-360 origin on peritoneal dialysis effluent (PDE) induced proliferation of primary human colon fibroblast from patient B (phCFB/B). phCFB/B cells were treated with PDE and with 3×10\hat{ }7 or 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. PDF, $$ p<0.01 3×10 vs.3×10 PDE.

FIG. 19. Effect of EV of Chlorella sp. MACC-360 origin on PDGF-B induced proliferation of primary human colon fibroblast from patient B (phCFB/B). phCFB/B cells were treated with PDGF-B and with 3×10\hat{ }7 or 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs.3×10 PDGF.

FIG. 20. Effect of EV of Chlorella sp. MACC-360 origin on peritoneal dialysis effluent (PDE) induced proliferation of primary human skin fibroblast from patient A (phSFB/A). phSFB/A cells were treated with PDE and with 3×10\hat{ }7 or 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ** p<0.01 3×10 vs. C (PBS), * p<0.05 3×10 vs. C (PBS), ##p<0.01 vs. PDF, $$ p<0.01 3×10 vs. PDE.

FIG. 21. Effect of EV of Chlorella sp. MACC-360 origin on PDGF-B induced proliferation of primary human skin fibroblast from patient A (phSFB/A). phSFB/A cells were treated with PDGF-B and with 3×10\hat{ }7 or 3×10\hat{ }8 particles/ml of EVs of Chlorella sp. MACC-360 origin for 24 hours. PBS and HCl were used as vehicle controls. The PDGF-B treatment induced proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ** p<0.01 3×10 vs. C (PBS), * p<0.05 3×10 vs. C (PBS), ##p<0.01 vs. C (HCl), $$ p<0.01 3×10 vs. PDGF.

FIG. 22. Effect of EV of Chlorella sp. MACC-360 origin on peritoneal dialysis (PDE) induced proliferation of primary human skin fibroblast from patient B (phSFB/B). phSFB/B cells were treated with PDE and with 3×10\hat{ }7 or 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. PDF, $$ p<0.01 3×10 vs.3×10 PDE.

FIG. 23. Effect of EV of Chlorella sp. MACC-360 origin on PDGF-B induced proliferation of primary human skin fibroblast from patient B (phSFB/B). phSFB/B cells were treated with PDGF-B and with 3×10\hat{ }7 or 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 24. Effect of EV of Chlorella sp. MACC-360 origin on peritoneal dialysis effluent (PDE) induced proliferation of human lung fibroblast cell line (MRC-5). MRC-5 cells were treated with PDE and with 3×10\hat{ }7 or 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. PDF, $$ p<0.01 3×10 vs. PDE, $ p<0.3×1005 vs. PDE.

FIG. 25. Effect of EV of Chlorella sp. MACC-360 origin on PDGF-B induced proliferation of human lung fibroblast cell line (MRC-5). MRC-5 cells were treated with PDGF-B and with 3×10\hat{ }7 or 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 26. Effect of EV of Chlorella sp. MACC-360 origin on peritoneal dialysis effluent (PDE) induced collagen production of primary human peritoneal fibroblast from patient A (phPFB/A). phPFB/A cells were treated with PDE and with 3×10\hat{ }7 or 3×10\hat{ }8 particles/ml of EVs of Chlorella sp. MACC-360 origin for 72 hours. PBS and peritoneal dialysis fluid (PDF) were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test. ##p<0.01 vs. PDF, $$p<0.01 3×10 vs. PDE.

FIG. 27. Effect of EV of Chlorella sp. MACC-360 origin on TGF-β induced collagen production of primary human peritoneal fibroblast from patient A (phPFB/A). phPFBs were treated with TGF-β and with 3×10\hat{ }7 or 3×10\hat{ }8 particles/ml of EVs of Chlorella sp. MACC-360 origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test. ##p<0.01 vs. C (HCl), $p<0.05 3×10 vs. TGF-B.

FIG. 28. Effect of EV of Chlorella sp. MACC-360 origin on peritoneal dialysis effluent (PDE) induced collagen production of primary human colon fibroblast from patient A (phCFB/A). phCFB/A cells were treated with PDE and with 3×10\hat{ }8 particles/ml of EVs of Chlorella sp. MACC-360 origin for 72 hours. PBS and peritoneal dialysis fluid (PDF) were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. PDF, $$ p<0.01 3×10 vs. PDE.

FIG. 29. Effect of EV of Chlorella sp. MACC-360 origin on TGF-β induced collagen production of primary human colon fibroblast from patient A (phCFB/A). phCFB/A cells were treated with TGF-β and with 3×10\hat{ }7 or 3×10\hat{ }8 particles/ml of EVs of Chlorella sp. MACC-360 origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$p<0.01 3×10 vs.3×10 TGF-B.

FIG. 30. Effect of EV of Chlorella sp. MACC-360 origin on peritoneal dialysis effluent (PDE) induced collagen production of primary human colon fibroblast from patient B (phCFB/B). phCFB/A cells were treated with PDE and with 3×10\hat{ }8 particles/ml of EVs of Chlorella sp. MACC-360 origin for 72 hours. PBS and peritoneal dialysis fluid (PDF) were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. PDF, $$ p<0.01 3×10 vs. PDE

FIG. 31. Effect of EV of Chlorella sp. MACC-360 origin on TGF-β induced collagen production of primary human colon fibroblast from patient B (phCFB/B). phCFB/B cells were treated with TGF-β and with 3×10\hat{ }7 or 3×10\hat{ }8 particles/ml of EVs of Chlorella sp. MACC-360 origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$p<0.01 3×10 vs.3×10 TGF-B.

FIG. 32. Effect of EV of Chlorella sp. MACC-360 origin on peritoneal dialysis effluent (PDE) induced collagen production of primary human skin fibroblast from patient A (phSFB/A). phSFB/A cells were treated with PDE and with 3×10\hat{ }7 or 3×10\hat{ }8 particles/ml of EVs of Chlorella sp. MACC-360 origin for 72 hours. PBS and peritoneal dialysis fluid (PDF) were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. €€ p<0.01 vs. C (PBS), * p<0.05 3×10 vs. C (PBS), ##p<0.01 vs. PDF, $$ p<0.01 3×10 vs.3×10 PDE.

FIG. 33. Effect of EV of Chlorella sp. MACC-360 origin on TGF-β induced collagen production of primary human skin fibroblast from patient A (phSFB/A). phSFB/A cells were treated with TGF-β and with 3×10\hat{ }7 or 3×10\hat{ }8 particles/ml of EVs of Chlorella sp. MACC-360 origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. * p<0.05 vs. C (PBS), ##p<0.01 vs. C (HCl), $$p<0.01 3×10 vs.3×10 TGF-B.

FIG. 34. Effect of EV of Chlorella sp. MACC-360 origin on peritoneal dialysis effluent (PDE) induced collagen production of primary human skin fibroblast from patient B (phSFB/B). phSFB/B cells were treated with PDE and with 3×10\hat{ }7 or 3×10\hat{ }8 particles/ml of EVs of Chlorella sp. MACC-360 origin for 72 hours. PBS and peritoneal dialysis fluid (PDF) were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. €€ p<0.01 vs. C (PBS), * p<0.05 3×10 vs. C (PBS), ** p<0.01 3×10 vs. C (PBS), ##p<0.01 vs. PDF, $$ p<0.01 3×10³×10 vs. PDE.

FIG. 35. Effect of EV of Chlorella sp. MACC-360 origin on TGF-β induced collagen production of primary human skin fibroblast from patient B (phSFB/B). phSFB/B cells were treated with TGF-β and with 3×10\hat{ }7 or 3×10\hat{ }8 particles/ml of EVs of Chlorella sp. MACC-360 origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. * p<0.05 3×10 vs. C (PBS), ** p<0.01 3×10 vs. C (PBS), ##p<0.01 vs. C (HCl), $$p<0.01 3×10 vs.3×10 TGF-B.

FIG. 36. Effect of EV of Chlorella sp. MACC-360 origin on TGF-β induced collagen production of lung fibroblast cells (MRC-5). MRC-5 cells were treated with TGF-B and with 3×10\hat{ }7 particle/ml of EVs of Chlorella sp. MACC-360 origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ** p<0.01 vs. C (PBS), ##p<0.01 3×10 vs. C (HCl), $p<0.05 3×10 vs. TGF-β

FIG. 37. Effect of EV of Chlorella sp. MACC-360 origin on IL10 gene expression of peripheral blood mononuclear cells (PBMC). PBMCs were treated with 3×10\hat{ }8 particles/ml concentration of EVs of Chlorella sp. MACC-360 origin for 24 hours. PBS was used as vehicle control. mRNA expression of IL10 was measured by real time RT-PCR and determined as a ratio of RPLP0 as internal control. The concentration of EVs in the figures refers to the concentration of the stock solution of the EVs used for treatment of the cells. Results are presented as mean±SD, dots represent individual values (n=6/group). Analysis of significance was performed by Mann-Whitney U-test. * p<0.05 3×10 vs. C (PBS).

FIG. 38. Effect of EV of Chlorella sp. MACC-360 origin on IL13 gene expression of peripheral blood mononuclear cells (PBMC). PBMCs were treated with 3×10\hat{ }8 particles/ml concentration of EVs of Chlorella sp. MACC-360 origin for 24 hours. PBS was used as vehicle control. mRNA expression of IL13 was measured by real time RT-PCR and determined as a ratio of RPLP0 as internal control. The concentration of EVs in the figures refers to the concentration of the stock solution of the EVs used for treatment of the cells. Results are presented as mean±SD, dots represent individual values (n=6/group). Analysis of significance was performed by unpaired t-test p=NS 3×10 vs. C (PBS).

FIG. 39. Effect of EV of Chlorella sp. MACC-360 origin on PDGFB gene expression of peripheral blood mononuclear cells (PBMC). PBMCs were treated with 3×10\hat{ }8 particles/ml concentration of EVs of Chlorella sp.

MACC-360 origin for 24 hours. PBS was used as vehicle control. mRNA expression of PDGFB was measured by real time RT-PCR and determined as a ratio of RPLP0 as internal control. The concentration of EVs in the figures refers to the concentration of the stock solution of the EVs used for treatment of the cells. Results are presented as mean±SD, dots represent individual values (n=6/group). Analysis of significance was performed by unpaired t-test ** p<0.01 3×10 vs. C (PBS).

FIG. 40. Effect of EV of Chlorella sp. MACC-360 origin on TGFB gene expression of peripheral blood mononuclear cells (PBMC). PBMCs were treated with 3×10\hat{ }8 particles/ml of EVs of Chlorella sp. MACC-360 origin for 24 hours. PBS was used as vehicle control. mRNA expression of TGFB was measured by real time RT-PCR and determined as a ratio of RPLP0 as internal control. The concentration of EVs in the figures refers to the concentration of the stock solution of the EVs used for treatment of the cells. Results are presented as mean±SD, dots represent individual values (n=6/group). Analysis of significance was performed by unpaired t-test U-test. p=NS 3×10 vs. C (PBS).

FIG. 41. Effect of EV of Chlorella sp. MACC-360 origin on IL6 gene expression of peripheral blood mononuclear cells (PBMC). PBMCs were treated with 3×10\hat{ }8 particles/ml of EVs of Chlorella sp. MACC-360 origin for 24 hours. mRNA expression of IL6 was measured by real time RT-PCR and determined as a ratio of RPLP0 as internal control. The concentration of EVs in the figures refers to the final concentration of the stock solution of the EVs used for treatment of the cells. Results are presented as mean±SD, dots represent individual values (n=6/group). After testing normality, analysis of significance was performed by unpaired t-test. ** p<0.01 vs. C (PBS).

FIG. 42. Effect of EV of Chlorella sp. MACC-360 origin on IL8 gene expression of peripheral blood mononuclear cells (PBMC). PBMCs were treated with 3×10\hat{ }8 particles/ml of EVs of Chlorella sp. MACC-360 origin for 24 hours. mRNA expression of IL8 was measured by real time RT-PCR and determined as a ratio of RPLP0 as internal control. The concentration of EVs in the figures refers to the final concentration of the stock solution of the EVs used for treatment of the cells. Results are presented as mean±SD, dots represent individual values (n=6/group). After testing normality, analysis of significance was performed by unpaired t-test. ** p<0.01 vs. C (PBS).

FIG. 43. Effect of EV of Chlorella sp. MACC-360 origin on MCP1 gene expression of peripheral blood mononuclear cells (PBMC). PBMCs were treated with 3×10\hat{ }8 particles/ml of EVs of Chlorella sp. MACC-360 origin for 24 hours. mRNA expression of MCP1 was measured by real time RT-PCR and determined as a ratio of RPLP0 as internal control. The concentration of EVs in the figures refers to the final concentration of the stock solution of the EVs used for treatment of the cells. Results are presented as mean±SD, dots represent individual values (n=6/group). After testing normality, analysis of significance was performed by unpaired t-test. ** p<0.01 vs. C (PBS).

FIG. 44. Effect of EV of Chlorella sp. MACC-360 origin on IL10 gene expression of peripheral blood mononuclear cells (PBMC) under physiological- and PHA (phytohemagglutinin) stimulated condition. PBMCs were treated with PHA and 3×10\hat{ }8 particles/ml of EVs of Chlorella sp. MACC-360 origin for 24 hours. mRNA expression of IL10 was measured by real time RT-PCR and determined as a ratio of RN18S as internal control. The concentration of EVs in the figures refers to the final concentration of the stock solution of the EVs used for treatment of the cells. Results are presented as mean±SD, dots represent individual values (n=6/group). After testing normality, analysis of significance was performed by unpaired t-test. ** p<0.01 vs. C (PBS), $$ p<0.01 vs. PHA.

FIG. 45. Effect of EV of Chlorella sp. MACC-360 origin on TGFB gene expression of peripheral blood mononuclear cells (PBMC). PBMCs were treated with PHA and 3×10\hat{ }8 particles/ml of EVs of Chlorella sp. MACC-360 origin for 24 hours. mRNA expression of TGFB was measured by real time RT-PCR and determined as a ratio of RN18S as internal control. The concentration of EVs in the figures refers to the final concentration of the stock solution of the EVs used for treatment of the cells. Results are presented as mean±SD, dots represent individual values (n=6/group). After testing normality, analysis of significance was performed by Mann-Whitney U-test. ##p<0.01 vs. C (PBS), $$ p<0.01 vs. PHA.

FIG. 46. Effect of EV of Chlorella sp. MACC-360 origin on IL13 gene expression of peripheral blood mononuclear cells (PBMC). PBMCs were treated with PHA and 3×10\hat{ }8 particles/ml EVs of Chlorella sp. MACC-360 origin for 24 hours. mRNA expression of IL13 was measured by real time RT-PCR and determined as a ratio of RN18S as internal control. The concentration of EVs in the figures refers to the final concentration of the stock solution of the EVs used for treatment of the cells. Results are presented as mean±SD, dots represent individual values (n=6/group). After testing normality, analysis of significance was performed by Mann-Whitney U-test. p=NS.

FIG. 47. Effect of EV of Chlorella sp. MACC-360 origin on IL-6 protein level of adenocarcinomic human alveolar basal epithelial cells (A549). A549 cells were treated with lipopolysaccharide (LPS), phytohemagglutinin (PHA), polyinosilic:polycytidilic acid [poly (I:C)] and 3×10\hat{ }8 particles/ml of EVs of Chlorella sp. MACC-360 origin for 24 hours. Protein level of IL-6 was measured by ELISA. The concentration of EVs in the figures refers to the final concentration of the stock solution of the EVs used for treatment of the cells. Results are presented as mean±SD, dots represent individual values (n=4/group). After testing normality, analysis of significance performed by unpaired t-test. ** p<0.01 vs. C (PBS), ##p<0.01 vs. C (PBS), $$ p<0.01 vs. PHA or Poly (I:C).

FIG. 48. Effect of EV of Chlorella sp. MACC-360 origin on IL-6 protein level of human skin fibroblast from patient A (phSFB/A). phSFB/A cells were treated with lipopolysaccharide (LPS), phytohemagglutinin (PHA), polyinosilic:polycytidilic acid [poly (I:C)] and 3×10\hat{ }8 particles/ml EVs of Chlorella sp. MACC-360 origin for 24 hours. Protein level of IL-6 was measured by ELISA. The concentration of EVs in the figures refers to the final concentration of the stock solution of the EVs used for treatment of the cells. Results are presented as mean±SD, dots represent individual values (n=4/group). After testing normality, analysis of significance performed by unpaired t-test. ** p<0.01 vs. C (PBS), ##p<0.01 vs. C (PBS), $$ p<0.01 vs. LPS or PHA or Poly (I:C).

FIG. 49. Effect of EV of Chlorella sp. MACC-360 origin on methylglyoxal (MGO) induced cytotoxicity of primary human omental mesothelial cells (HPMC). HPMCs were treated with 600 μM and 700 μM MGO and with 3×10\hat{ }7 or 3×10\hat{ }8 particles/ml of EVs of Chlorella sp. MACC-360 origin for 24 hours. PBS was used as vehicle control. Cell viability was determined by MTT assay. The concentration of EVs in the figures refers to the concentration of the stock solution of the EVs used for treatment of the cells. Results are presented as mean±SD, dots represent individual values (n=5/group). Analysis of significance was performed by Mann-Whitney U3×103×10^{3×10}-test. *##p<0.01 vs. C (PBS), $$ p<0.01 vs. corresponding MGO.

FIG. 50. Effect of EV of Chlorella sp. MACC-360 origin on methylglyoxal (MGO) induced cytotoxicity of primary human omental mesothelial cells (HPMC). HPMCs were treated with 700 μM MGO and with 3×10\hat{ }7 or 3×10\hat{ }8 particles/ml of EVs of Chlorella sp. MACC-360 origin for 24 hours. PBS was used as vehicle control.

Cytotoxicity was determined by LDH assay. The concentration of EVs in the figures refers to the concentration of the stock solution of the EVs used for treatment of the cells. Results are presented as mean±SD, dots represent individual values (n=5/group). Analysis of significance was performed by Mann-Whitney U-test. ** p<0.01 EV 3×10 vs. C (PBS), ##p<0.01 vs. C (PBS), $$ p<0.01 3×10 vs. 700 μM MGO.

FIG. 51. Effect of EV of Chlorella sp. MACC-360 origin on methylglyoxal (MGO) induced cytotoxicity of primary human colon fibroblast from patient A (phCFB/A). phCFB/A cells were treated with 100 μM MGO and with 3×10\hat{ }8 particles/ml of EVs of MACC-360 origin for 24 hours. PBS was used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=5/group). After testing normality analysis of significance was performed by Mann-Whitney U-test. #p<0.05 vs. C (PBS), $$ p<0.01 vs. 100 μM MGO.

FIG. 52. Effect of EV of Chlorella sp. MACC-360 origin on 3,4-Dideoxyglucosone-3-ene (DGE) induced cytotoxicity of primary human colon fibroblast from patient A (phCFB/A). phCFB/A cells were treated with 75 μM DGE and with 3×10\hat{ }8 particles/ml EVs of MACC-360 origin for 24 hours. PBS was used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=5/group). After testing normality analysis of significance was performed by Mann-Whitney U-test. ##p<0.01 vs. C (PBS), $$ p<0.01 vs. 75 μM DGE.

FIG. 53. Effect of EV of Chlorella sp. MACC-360 origin on 5-Hydroxymethyl-2-furaldehyde (HMF) induced cytotoxicity of primary human colon fibroblast from patient A (phCFB/A). phCFB/A cells were treated with 10 mM HMF and with 3×10\hat{ }8 particles/ml of EVs of MACC-360 origin for 24 hours. PBS was used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=5/group). After testing normality analysis of significance was performed by Mann-Whitney U-test. ##p<0.01 vs. C (PBS), $$ p<0.01 vs. 10 mM HMF.

FIG. 54. Effect of EV of Chlorella sp. MACC-360 origin on methylglyoxal (MGO) induced cytotoxicity of primary human skin fibroblasts from patients A (phSFB/A). phSFB/A cells were treated with 300 μM MGO and with 3×10\hat{ }8 particles/ml of EVs of MACC-360 origin for 24 hours. PBS was used as vehicle controls. Cell viability was determined by MTT assay. The concentration of EVs in the figures refers to the final concentration of the stock solution of the EVs used for treatment of the cells. Results are presented as mean±SD, dots represent individual values (n=5/group). After testing normality analysis of significance was performed by Mann-Whitney U-test. ** p<0.01 vs. C (PBS), ##p<0.01 vs. C (PBS), $$ p<0.01 vs. 300 μM MGO.

FIG. 55. Effect of EV of Chlorella sp. MACC-360 origin on methylglyoxal (MGO) induced cytotoxicity of primary human skin fibroblasts from patient A (phSFB/A). phSFB/A cells were treated with 300 μM MGO and with 3×10\hat{ }8 particles/ml of EVs of MACC-360 origin for 24 hours. PBS was used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=5/group). After testing normality analysis of significance was performed by Mann-Whitney U-test. * p<0.05 vs. C (PBS), ##p<0.01 vs. C (PBS), $$ p<0.01 vs. 300 μM MGO.

FIG. 56. Effect of EV of Chlorella sp. MACC-360 origin on 5-Hydroxymethyl-2-furaldehyde (HMF) induced cytotoxicity of primary human skin fibroblasts from patient A (phSFB/A). phSFB/A cells were treated with 5 mM HMF and with 3×10\hat{ }8 particles/ml of EVs of MACC-360 origin for 24 hours. PBS was used as vehicle controls.

Cell viability was determined by MTT assay. The concentration of EVs in the figures refers to the final concentration of the stock solution of the EVs used for treatment of the cells. Results are presented as mean±SD, dots represent individual values (n=5/group). After testing normality analysis of significance was performed by Mann-Whitney U-test. ** p<0.01 vs. C (PBS), ##p<0.01 vs. C (PBS), $ p<0.05 vs. 5 mM HMF.

FIG. 57. Effect of EV of Chlorella sp. MACC-360 origin on methylglyoxal (MGO) induced cytotoxicity of primary human umbilical cord endothelial cells (HUVEC). HUVEC cells were treated with 500 μM MGO and with 3×10\hat{ }8 particles/ml of EVs of Chlorella sp. MACC-360 origin for 24 hours. PBS was used as vehicle control. Cell viability was determined by MTT assay. The concentration of EVs in the figures refers to the concentration of the stock solution of the EVs used for treatment of the cells. Results are presented as mean±SD, dots represent individual values (n=5/group). Analysis of significance was performed by Mann-Whitney U-test. ##p<0.01 vs. C (PBS), $$ p<0.01 3×10 vs. MGO.

FIG. 58. Effect of EV of Chlorella sp. MACC-360 origin on methylglyoxal (MGO) induced cytotoxicity of primary human umbilical cord endothelial cells (HUVEC). HUVEC cells were treated with 500 μM MGO and with 3×10\hat{ }8 particles/ml of EVs of Chlorella sp. MACC-360 origin for 24 hours. PBS was used as vehicle control. Cytotoxicity was determined by LDH assay. The concentration of EVs in the figures refers to the concentration of the stock solution of the EVs used for treatment of the cells. Results are presented as mean±SD, dots represent individual values (n=5/group). Analysis of significance was performed by Mann-Whitney U-test. ##p<0.01 vs. C (PBS), $$ p<0.01 3×10 vs. MGO.

FIG. 59. Effect of EVs of Chlorella sp. MACC-360 origin on cell migration of primary human colon fibroblast from patient A (phCFB/A). phCFB/A cells were treated with 10 ng/ml EGF and with 3×10\hat{ }8 particles of EVs of Chlorella sp. MACC-360 origin. PBS was used as vehicle control. Cell migration was determined by TAS assay. The concentration of EVs in the figures refers to the concentration of the stock solution of the EVs used for treatment of the cells. Results are presented as mean±SD (n=6/group). Analysis of significance was performed by two-way ANOVA. $$ p<0.01 EGF vs. EGF+EV 3×10 at the concerning time.

FIG. 60. Effect of EVs of Chlorella sp. MACC-360 origin on cell migration of primary skin fibroblast from patient A (phSFB/A). phSFB/A cells were treated with 10 ng/ml EGF and with 3×10\hat{ }8 particles/ml of EVs of Chlorella sp. MACC-360 origin. PBS was used as vehicle control. Cell migration was determined by TAS assay. The concentration of EVs in the figures refers to the concentration of the stock solution of the EVs used for treatment of the cells. Results are presented as mean±SD (n=6/group). Analysis of significance was performed by two-way ANOVA. $$ p<0.01 EGF vs. EGF+EV 3×10 at the concerning.

FIG. 61. Effect of EV of Chlorella sp. MACC-360 origin on PDGF-B induced proliferation of adenocarcinomic human alveolar basal epithelial cells (A549). A549 cells were treated with PDGF-B and with 3×10\hat{ }8 or 3×10\hat{ }9 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ** p<0.01 3×10 vs. C (PBS), ##p<0.01 3×10 vs. C (HCl), $$ p<0.01 3×10³×10 vs. PDGF.

FIG. 62. Effect of EV of Chlorella sp. MACC-360 origin on PDGF-B induced proliferation of human colorectal adenocarcinoma cell line (HT-29). HT-29 cells were treated with PDGF-B and with 3×10\hat{ }8 or 3×10\hat{ }9 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ** p<0.01 3×10 vs. C (PBS), ##p<0.01 3×10 vs. C (HCl), $$ p<0.01 3×10³×10 vs. PDGF.

FIG. 63. Effect of EV of Chlorella sp. MACC-360 origin on PDGF-B induced proliferation of colon carcinoma cell line (Caco-2). Caco-2 cells were treated with PDGF-B and with 3×10\hat{ }8 or 3×10\hat{ }9 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ** p<0.01 3×10 vs. C (PBS), ##p<0.01 3×10 vs. C (HCl), $$ p<0.01 3×10³×10 vs. PDGF.

FIG. 64. Effect of EVs of Chlorella sp. MACC-360 origin on cell migration of colon carcinoma cell line (Caco-2). phSFB/A cells were treated with EGF or peritoneal dialysis effluent (PDE) and with 3×10\hat{ }8 particles/ml of EVs of Chlorella sp. MACC-360 origin. PBS and peritoneal dialysis fluid (PDF) were used as vehicle controls. Cell migration was determined by TAS assay. The concentration of EVs in the figures refers to the concentration of the stock solution of the EVs used for treatment of the cells. Results are presented as mean±SD (n=6/group). Analysis of significance was performed by two-way ANOVA. $$ p<0.01 PDE vs. PDE+EV 3×10³×10 at the concerning time.

FIG. 65. Effect of EV of Chlorella sp. MACC-360 origin on peritoneal dialysis effluent (PDE) induced proliferation of human breast carcinoma cell line (Hs578-T). Hs578-T cells were treated with PDE and with 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the concentration of the stock solution of the EVs used for treatment of the cells. Results are presented as mean±SD, dots represent individual values (n=6/group). Analysis of significance was performed by unpaired t-test. ** p<0.01, ##p<0.01 vs. C (PDF), $$ p<0.01 vs. PDE.

FIG. 66. Effect of EV of Chlorella sp. MACC-360 origin on cell death of peritoneal dialysis effluent (PDE) treated human breast carcinoma cell line (Hs578-T). Hs578-T cells were treated with PDE and with 3×10\hat{ }8 particles/ml of EVs of Chlorella sp. MACC-360 origin for 24 hours. PBS and peritoneal dialysis fluid (PDF) were used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=6/group). Analysis of significance was performed by unpaired t-test. p=NS.

FIG. 67. Effect of EV of Chlorella sp. MACC-360 origin on peritoneal dialysis effluent (PDE) induced proliferation of human large cell lung carcinoma cell line (LCLC-103H). LCLC-103H cells were treated with PDE and with 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the final concentration of the stock solution of the EVs used for treatment of the cells. Results are presented as mean±SD, dots represent individual values (n=6/group). Analysis of significance was performed by unpaired t test and Mann-Whitney test. ** p<0.01 vs. C (PBS), ##p<0.01 vs. C (PDF), $$ p<0.01 vs. PDE.

FIG. 68. Effect of EV of Chlorella sp. MACC-360 origin on cell death of peritoneal dialysis effluent (PDE) treated human large cell lung carcinoma cell line (LCLC-103H). LCLC-103H were treated with PDE and with 3×10\hat{ }8 particles/ml of EVs of Chlorella sp. MACC-360 origin for 24 hours. PBS and peritoneal dialysis fluid (PDF) were used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=6/group). Analysis of significance was performed by unpaired t test. p=NS.

FIG. 69. Effect of EV of Chlorella sp. MACC-360 origin on peritoneal dialysis effluent (PDE) induced proliferation of human hepatocellular carcinoma cell line (HEPG2). HEPG2 cells were treated with PDE and with 3×10\hat{ }8 particles/ml of EVs of Chlorella sp. MACC-360 origin for 24 hours. PBS and peritoneal dialysis fluid (PDF) were used as vehicle controls. Cell proliferation was determined by MTT assay. The concentration of EVs in the figures refers to the final concentration of the stock solution of the EVs used for treatment of the cells. Results are presented as mean±SD, dots represent individual values (n=6/group). Analysis of significance was performed by unpaired t test. ** p<0.01 vs. C (PBS), ##p<0.01 vs. C (PDF), $$ p<0.01 vs. PDE.

FIG. 70. Effect of EV of Chlorella sp. MACC-360 origin on cell death of peritoneal dialysis effluent (PDE) treated human hepatocellular carcinoma cell line (HEPG2). HEPG2 cells were treated with PDE and with 3×10\hat{ }8 particles/ml EVs of Chlorella sp. MACC-360 origin for 24 hours. PBS and peritoneal dialysis fluid (PDF) were used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=6/group). Analysis of significance was performed by unpaired t test and Mann-Whitney test. ** p<0.01 vs. C (PBS)

FIG. 71. Effect of EV of Chlorella sp. MACC-360 origin on the peritoneal thickness in a chlorhexidin-gluconate (CG) induced peritoneal fibrosis model of mice. Peritoneal fibrosis was induced by daily intraperitoneal (i.p.) injections of CG for 7 days. 1*10\hat{ }9 particles of EVs of Chlorella sp. MACC-360 were i.p injected twice a week.

Extent of fibrosis (blue) in peritoneal sections was visualized by microscopy after Masson trichome staining (−1), then submesothelial thickness was quantified graphically (−2). Images were taken with 20× objective. The concentration of EVs in the figures refers to the concentration of the stock solution of the EVs used for treatment of the mice. Results are presented as mean±SD, dots represent individual values. Analysis of significance was performed by Mann-Whitney U-test. ##p<0.01 vs. C (PBS), $$ p<0.01 3×10 vs. CG.

FIG. 72. Average size/size distribution of EVs isolated from Chlorella sp. MACC-360 using different isolation methods (−1: ultracentrifugation; −2: Amicon and SEC) was measured by dynamic light scattering (DLS):

ucf_10xdil in PBS

| Date: | May 3, 2022 4:10:20 PM | | Temperature: | 27.9° | C. | Data Filter: | Default |
|---|---|---|---|---|---|---|---|
| Solvent: | PBS | | Solute: | — | | Laser: | 100% |
| Intensity: | 408,453 | counts/s | Intercept: | 0.619 | | Attenuator: | 100% |
| Z - Av. Diameter: | 182.14 | nm | Std. Deviation: | 199.14 | nm | MW model: | Globular Proteins |

| | | | | | |
|---|---|---|---|---|---|
| Polydispersity: | 109.33% | Pd. Index: | 1.195 | Remark: | — |

| Peak | Mean Dh | Mode Dh | Std. Dev. | Polydisp. | Est. MW. | Intensity | Mass | Volume | Number |
|---|---|---|---|---|---|---|---|---|---|
| # | (nm) | (nm) | (nm) | (%) | (kDa) | (%) | (%) | (%) | (%) |
| 1 | 235.26 | 274.55 | 126.94 | 53.96 | Out of Range | 100.00 | 100.00 | 100.00 | 100.00 |

amsecfr1_10xdil in PBS

| Date: | May 3, 2022 3:27:46 PM | | Temperature: | 19.98° | C. | Data Filter: | Default |
|---|---|---|---|---|---|---|---|
| Solvent: | PBS | | Solute: | — | | Laser: | 100% |
| Intensity: | 123,655 | counts/s | Intercept: | 0.602 | | Attenuator: | 100% |
| Z - Av. Diameter: | 205.07 | nm | Std. Deviation: | 2.29 | nm | MW model: | Globular Proteins |

| | | | | | |
|---|---|---|---|---|---|
| Polydispersity: | 1.12% | Pd. Index: | 0.000 | Remark: | — |

| Peak | Mean Dh | Mode Dh | Std. Dev. | Polydisp. | Est. MW. | Intensity | Mass | Volume | Number |
|---|---|---|---|---|---|---|---|---|---|
| # | (nm) | (nm) | (nm) | (%) | (kDa) | (%) | (%) | (%) | (%) |
| 1 | 132.65 | 137.26 | 96.73 | 72.92 | Out of Range | 100.00 | 100.00 | 100.00 | 100.00 |

ucf_10xdil in PBS

| Date: | May 3, 2022 4:10:20 PM | | Temperature: | 27.9° | C. | Data Filter: | Default |
|---|---|---|---|---|---|---|---|
| Solvent: | PBS | | Solute: | — | | Laser: | 100% |
| Intensity: | 408,453 | counts/s | Intercept: | 0.619 | | Attenuator: | 100% |
| Z - Av. Diameter: | 182.14 | nm | Std. Deviation: | 199.14 | nm | MW model: | Globular Proteins |

| | | | | | |
|---|---|---|---|---|---|
| Polydispersity: | 109.33% | Pd. Index: | 1.195 | Remark: | — |

| Peak | Mean Dh | Mode Dh | Std. Dev. | Polydisp. | Est. MW. | Intensity | Mass | Volume | Number |
|---|---|---|---|---|---|---|---|---|---|
| # | (nm) | (nm) | (nm) | (%) | (kDa) | (%) | (%) | (%) | (%) |
| 1 | 235.26 | 274.55 | 126.94 | 53.96 | Out of Range | 100.00 | 100.00 | 100.00 | 100.00 |

FIG. 73. Effect of EV of Chlorella sp. MACC-360 origin isolated by ultrafiltration with tangential flow flow filtration (TFF EV) on PDGF-B induced proliferation of primary human skin fibroblast from patient A (phSFB/A). phSFB/A cells were treated with PDGF-B and with 3×10\hat{ }8 particles ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 74. Effect of EV of Chlorella sp. MACC-360 origin isolated by tangential flow filtration with size-exclusion chromatography (TFF+SEC EV) on PDGF-B induced proliferation of primary human skin fibroblast from patient A (phSFB/A). phSFB/A cells were treated with PDGF-B and with 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0. vs. C (HCl), $$ p<0.01 3×10 vs. PDGF.

FIG. 75. Effect of EV of Chlorella sp. MACC-360 origin isolated by ultracentrifugation (UCF EV) on PDGF-B induced proliferation of primary human skin fibroblast from patient A (phSFB/A). phSFB/A cells were treated with PDGF-B and with 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 76. Effect of EV of Chlorella sp. MACC-360 origin isolated by ultrafiltration using Amicon filter (AMICON EV) on PDGF-B induced proliferation of primary human skin fibroblast from patient A (phSFB/A). phSFB/A cells were treated with PDGF-B and with 3×10\hat{ }8 particles/ml of EVs of Chlorella sp. MACC-360 origin for 24 hours. PBS and HCl were used as vehicle controls. Cell proliferation was determined by MTT assay.

The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 PDGF vs. C (HCl), $$ p<0.01 PDGF+3×10\hat{ }8 particles/ml AMICON EV vs. PDGF.

FIG. 77. Effect of EV of Chlorella sp. MACC-360 origin isolated by ultrafiltration using Amicon filter with size-exclusion chromatography (AM+SEC EV) on PDGF-B induced proliferation of primary human skin fibroblast from patient A (phSFB/A). phSFB/A cells were treated with PDGF-B and with 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t test or Mann-Whitney U-test. ##p<0.01 vs. C (HCl), $ p<0.05 3×10 vs. PDGF.

FIG. 78. Effect of EV of Chlorella sp. MACC-360 origin isolated by dialization with size-exclusion chromatography (DIAL+SEC EV) on PDGF-B induced proliferation of primary human skin fibroblast from patient A (phSFB/A). phSFB/A cells were treated with PDGF-B and with 3×10\hat{ }8 particles ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 79. Effect of EV of Chlorella sp. MACC-360 origin isolated by sonication/extraction (EXTR EV) on PDGF-B induced proliferation of primary human skin fibroblast from patient A (phSFB/A). phSFB/A cells were treated with PDGF-B and with 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 80. Effect of EV of Chlorella sp. MACC-360 origin isolated by sonication/extraction with size-exclusion chromatography (EXTR+SEC EV) on PDGF-B induced proliferation of primary human skin fibroblast from patient A (phSFB/A). phSFB/A cells were treated with PDGF-B and with 3×10\hat{ }8 particles/ml of EVs of Chlorella sp. MACC-360 origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF

FIG. 81. EVs of Parachlorella kessleri origin (arrows) were visualized by transmission electron microscopy (TEM) after negative staining with uranyl acetate (−1). Average size/size distribution of EVs of Parachlorella kessleri origin and their particle numbers was measured by dynamic light scattering (DLS)

C38 Amicon SEC_FR2

| Date: | Apr. 7, 2022 4:31:29 PM | | Temperature: | 20° | C. | Data Filter: | Default |
|---|---|---|---|---|---|---|---|
| Solvent: | PBS | | Solute: | — | | Laser: | 100% |
| Intensity: | 112,418 | counts/s | Intercept: | 0.576 | | Attenuator: | 100% |
| Z - Av. Diameter: | 178.06 | nm | Std. Deviation: | 109.29 | nm | MW model: | Globular Proteins |

| | | | | | |
|---|---|---|---|---|---|
| Polydispersity: | 61.38% | Pd. Index: | 0.377 | Remark: | — |

| Peak | Mean Dh | Mode Dh | Std. Dev. | Polydisp. | Est. MW. | Intensity | Mass | Volume | Number |
|---|---|---|---|---|---|---|---|---|---|
| # | (nm) | (nm) | (nm) | (%) | (kDa) | (%) | (%) | (%) | (%) |
| 1 | 176.92 | 174.73 | 73.61 | 41.60 | Out of Range | 100.00 | 100.00 | 100.00 | 100.00 |

and multiple resistive pulse sensing (MRPS).

| Run Details | |
|---|---|
| Cartridge class: | TS400 |
| Mold ID: | P12.4.2 |
| Cartridge Box Date: | 210303 |
| Sample Description: | 05_BC360_2_10xdil |
| Cal particle size (nm): | 1.0 |
| Cal particle concentration (/mL): | 1.00E+00 |
| Analysis | |
| Size range min (nm): | 65.0 |
| Size range max (nm): | 400.0 |
| Total concentration (/mL): | 7.94E+08 |
| Number of particles detected: | 2247 |
| Statistical error in concentration (%): | 2.4% |
| D10 (nm): | 65.0 |
| D50 (nm): | 75.1 |
| D90 (nm): | 108.6 |
| Data processing user name: | mrps |
| Measurement Parameters | |
| Measurement date: | 20210528 |
| Measurement user name: | mrps |
| Acquisition time (s): | 620.0 |
| Measured volume (nL) | 2.83E+00 |
| Diameter scaling factor | 1.000 |
| Concentration scaling factor | 1.000 |

FIG. 82. Effect of EV of Parachlorella kessleri origin on PDGF-B induced proliferation of primary human colon fibroblast from patient A (phCFB/A). phCFB/A cells were treated with PDGF-B and with 3×10\hat{ }8 particles/ml of EVs of Parachlorella kessleri origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=6/group). Analysis of significance was performed by unpaired t-test or Mann-Whitney U-test. ** p<0.01 3×10 vs. C (PBS), ##p<0.01 vs. C (HCl), $$ p<0.01 3×10 vs. PDGF.

FIG. 83. Effect of EV of Parachlorella kessleri origin on TGF-β induced collagen production of primary human colon fibroblast from patient A (phCFB/A). phCFB/A cells were treated with TGF-β and with 3×10\hat{ }8 particles/ml of EVs of Parachlorella kessleri origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=6/group). Analysis of significance was performed by unpaired t-test. ##p<0.01 vs. C (HCl), $$p<0.01 3×10 vs. TGF-β.

FIG. 84. Effect of EV of Parachlorella kessleri origin on TGF-β induced collagen production of primary human skin fibroblast from patient A (phSFB/A). phSFB/A cells were treated with TGF-β and with 3×10\hat{ }8 particles/ml of EVs of Parachlorella kessleri origin for 72 hours. PBS and HCl were used as vehicle controls. Collagen accumulation was determined by Sirius Red assay. The concentration of EVs in the figures refers to the concentration of the stock solution of the EVs used for treatment of the cells. Results are presented as mean±SD, dots represent individual values (n=6/group). Analysis of significance was performed by unpaired t-test. ##p<0.01 vs. C (HCl), $$ p<0.01 3×10 vs. TGF-B.

FIG. 85. EVs of Spirulina platensis (−1, −2) as well as Chlorella pyrenoidosa (−3, −4) origin (arrows) were visualized by transmission electron microscopy (TEM) after negative staining with uranyl acetate (−1, −3). Average size/size distribution of EVs was measured by dynamic light scattering (DLS) (−2, −4):

SP lio FR1_10xdil in PBS

| Date: | May 5, 2022 10:31:18 AM | | Temperature: | 20° | C. | Data Filter: | Default |
|---|---|---|---|---|---|---|---|
| Solvent: | PBS | | Solute: | — | | Laser: | 100% |
| Intensity: | 538,301 | counts/s | Intercept: | 0.653 | | Attenuator: | 40% |
| Z - Av. Diameter: | 275.51 | nm | Std. Deviation: | 235.57 | nm | MW model: | Globular Proteins |

| | | | | | |
|---|---|---|---|---|---|
| Polydispersity: | 85.50% | Pd. Index: | 0.731 | Remark: | — |

| Peak | Mean Dh | Mode Dh | Std. Dev. | Polydisp. | Est. MW. | Intensity | Mass | Volume | Number |
|---|---|---|---|---|---|---|---|---|---|
| # | (nm) | (nm) | (nm) | (%) | (kDa) | (%) | (%) | (%) | (%) |
| 1 | 281.55 | 306.46 | 80.76 | 28.68 | Out of Range | 100.00 | 100.00 | 100.00 | 100.00 |

CV lio FR1_10xdil in PBS

| Date: | May 5, 2022 10:28:35 AM | | Temperature: | 20° | C. | Data Filter: | Default |
|---|---|---|---|---|---|---|---|
| Solvent: | PBS | | Solute: | — | | Laser: | 100% |
| Intensity: | 537,834 | counts/s | Intercept: | 0.616 | | Attenuator: | 100% |
| Z - Av. Diameter: | 224.17 | nm | Std. Deviation: | 124.12 | nm | MW model: | Globular Proteins |

| | | | | | |
|---|---|---|---|---|---|
| Polydispersity: | 55.37% | Pd. Index: | 0.307 | Remark: | — |

| Peak | Mean Dh | Mode Dh | Std. Dev. | Polydisp. | Est. MW. | Intensity | Mass | Volume | Number |
|---|---|---|---|---|---|---|---|---|---|
| # | (nm) | (nm) | (nm) | (%) | (kDa) | (%) | (%) | (%) | (%) |
| 1 | 208.35 | 261.01 | 88.14 | 42.30 | Out of Range | 100.00 | 100.00 | 100.00 | 100.00 |

Protein and lipid content and ratio was analysed by Fourier-transform infrared spectroscopy (FTIR) (−5).

FIG. 86. Effect of EV of Spirulina platensis origin isolated by ultrafiltration using Amicon filter (AMICON EV) on PDGF-B induced proliferation of primary human skin fibroblast from patient A (phSFB/A). phSFB/A cells were treated with PDGF-B and with 3×10\hat{ }8 particle/ml of EVs of Spirulina platensis origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.3×10 vs. PDGF.

FIG. 87. Effect of EV of Spirulina sp. origin isolated by ultrafiltration using Amicon filter with size-exclusion chromatography (AM+SEC EV) on PDGF-B induced proliferation of primary human skin fibroblast from patient A (phSFB/A). phSFB/A cells were treated with PDGF-B and with 3×10\hat{ }8 particle/ml of EVs of Spirulina platensis origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 88. Effect of EV of Chlorella pyrenoidosa origin isolated by ultrafiltration using Amicon filter (AMICON EV) on PDGF-B induced proliferation of primary human skin fibroblast from patient A (phSFB/A). phSFB/A cells were treated with PDGF-B and with 3×10\hat{ }8 particle/ml of EVs of Chlorella pyrenoidosa origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF

FIG. 89. Effect of EV of Chlorella pyrenoidosa origin isolated by ultrafiltration using Amicon filter with size-exclusion chromatography (AM+SEC EV) on PDGF-B induced proliferation of primary human skin fibroblast from patient A (phSFB/A). phSFB/A were treated with PDGF-B and with 3×10\hat{ }8 particle/ml of EVs of Chlorella pyrenoidosa origin for 24 hours. PBS and HCl were used as vehicle controls. Cell proliferation was determined by MTT assay. The concentration of EVs in the figures refers to the concentration of the stock solution of the EVs used for treatment of the cells. Results are presented as mean±SD, dots represent individual values (n=5/group). Analysis of significance was performed by unpaired t-test or Mann-Whitney U-test. ##p<0.01 vs. C (HCl), $$ p<0.01 3×10 vs. PDGF.

FIG. 90. In vivo internalization of EVs of Chlorella sp. MACC-360 origin into the liver. 10\hat{ }9 particles of the DiI labelled EVs (red) was intravenously administered to the tail vain of C57/BL6 J mice. After 24 hours, mice were sacrificed and the liver was harvested. Images were taken with 100×objective.

FIG. 91-1. In vivo internalization of EVs of MACC-1 (Chlorella vulgaris) origin into the visceral peritoneum. 10\hat{ }9 particles of the DiI labelled EVs (red) was intraperitoneally administered to C57/BL6 J mice. After 24 hours, mice were sacrificed and the visceral peritoneum was harvested. Cell nuclei were stained with DAPI (blue). Images were taken with 20× objective.

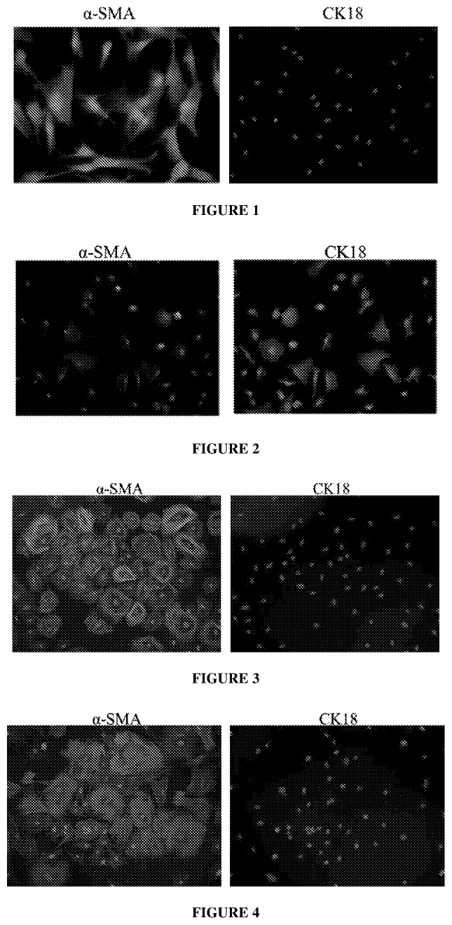

FIG. 91-2. In vivo internalization of EVs of MACC-3 (Chlorella pyrenoidosa) origin into the visceral peritoneum. 10\hat{ }9 particles of the DiI labelled EVs (red) was intraperitoneally administered to C57/BL6 J mice. After 24 hours,h mice were sacrificed and the visceral peritoneum was harvested. Cell nuclei were stained with DAPI (blue). Images were taken with 20× objective.

FIG. 92. In vivo internalization of EVs of MACC-1 (Chlorella vulgaris), MACC-3 (Chlorella pyrenoidosa) and MACC-1023 (Hormidiospora verrucosa) origin into the peritoneal lavage cells. 10\hat{ }9 particles of the DiI labelled EVs (red) was intraperitoneally administered to C57/BL6 J mice. After 24 hours, mice were sacrificed and peritoneal lavage was collected. Cell nuclei were stained with DAPI (blue). FIG. 93. In vivo internalization of EVs of Chlorella sp. MACC-360 origin into the skin. 10\hat{ }9 particles of the DiI labelled EVs (red) was intracutaneously administered to C57/BL6 J mice. After 24 hours, mice were sacrificed and the skin was harvested. Images were taken with 20× objective.

FIG. 94. Particle numbers of EVs of Chlorella sp. MACC-360 origin was measured by nanoparticle tracking analysis (NTA) (−1) and multiple resistive pulse sensing (MRPS) (−2) to determine the exact dose of it for oral administration (gavage) to C57/BL6 mice.

Table to FIG. 94-1 shows PARTICLEMETRIX measurement, i.e. Electrophoresis & Brownian Motion Video Analysis Laser Scattering Microscopy at Electrolyte: PBS, Temperature: 25.04° C. sensed, pH 7 0 entered, Conductivity. 15000.00 pS/cm sensed.

### Result (Sizes in Nm)

| | Number | Concentration | Volume |
|---|---|---|---|
| Median (X50) | 164 6 | 164 6 | 368 5 |
| Span | 98 9 | 98 8 | 278 8 |

| | |
|---|---|
| Concentration: | 3 0E*7 Particles t mL |
| Dilution Factor: | 1000 |
| Original Concentration: | 3 0E*10 Particles/mL |

TABLE
FIG. 94-2

| Spectradyne Run Report | |
|---|---|
| Created at: | 2023-02-24 16:33:45.689000 |
| Created by: | mrps |
| Data fie name: | 02 MACC 360 FRI 2Q230224 ct01JhS |
| Run Details | |
| Cartridge class: | C400 |
| Mold ID: | 109N |
| Cartridge Box Date: | 221104 |
| Sample Description: | 02_MACC_360_FR1. 10xd il 0.03% |
| | TPBS |
| Cal particle size (nm): | 1.0 |
| Cal particle concentration (/mL): | 1.00E +00 |
| Analysis | |
| Size range min (nm): | 65.0 |
| Size range max (nm): | 400.0 |
| Total concentration (/mt): | 1.52E+10 |
| Number of particles detected: | 1983 |
| Statistical error in concentration | 2.3% |
| (%): | |
| D10 (nm): | 68.4 |
| D50 (nm): | 78.4 |
| D90 (nm): | 111.9 |
| Data processing user name: | mrps |
| Measurement Parameters | |
| Measurement date: | 20230224 |
| Measurement user name: | mrps |
| Acquisition time (s): | 40.0 |
| Measured volume {nL) | 1.31E+0.1 |
| Diameter scaling factor | 1.000 |
| Concentration scaling factor | 1.000 |

FIG. 95. Effect of EV of Chlorella sp. MACC-360 origin on the disease activity index (DAI) of dextrane sodium sulphate (DSS) induced colitis model of mice. Briefly, C57BL/6J mice received 2.5% DSS in their drinking water for 7 days (DSS). Furthermore, mice were treated by daily oral gavage of 10\hat{ }9 particles of EVs of Chlorella sp. MACC-360 (DSS+MACC-360). Changes in DAI were monitored daily during the whole experiment. Results are presented as mean±SD. Analysis of significance was performed by two-way ANOVA. * p<0.05 DSS+MACC-360 EV vs. DSS at the concerning day.

FIG. 96. Effect of EV of Chlorella sp. MACC-360 origin using autotroph (auto) and heterotroph (het) cultivation on peritoneal dialysis effluent (PDE) induced proliferation of human hepatocellular carcinoma cell line (HEPG2). HEPG2 cells were treated with PDE and with 3×10\hat{ }8 particles/ml of EVs of Chlorella sp. MACC-360 origin—from autotroph and heterotroph cultivation—for 24 hours. PBS and peritoneal dialysis fluid (PDF) were used as vehicle controls. Cell proliferation was determined by MTT assay. The concentration of EVs in the figures refers to the final concentration of the stock solution of the EVs used for treatment of the cells. Results are presented as mean±SD, dots represent individual values (n=6/group). Analysis of significance was performed by unpaired t-test. ** p<0.01 vs. C (PBS), ##p<0.01 vs. C (PDF), $$ p<0.01 vs. PDE, $ p<0.05 vs. PDE.

FIG. 97. Effect of EV of Chlorella sp. MACC-360 origin using autotroph (auto) and heterotroph (het) cultivation on cell death of peritoneal dialysis effluent (PDE) treated human hepatocellular carcinoma cell line (HEPG2). HEPG2 cells were treated with PDE and with 3×10\hat{ }8 particles/ml of EVs of Chlorella sp. MACC-360 origin—from autotroph and heterotroph cultivation—for 24 hours. PBS and peritoneal dialysis fluid (PDF) were used as vehicle controls. Cytotoxicity was determined by LDH assay. The concentration of EVs in the figures refers to the final concentration of the stock solution of the EVs used for treatment of the cells. Results are presented as mean+SD, dots represent individual values (n=6/group). Analysis of significance was performed by unpaired t-test and Mann-Whitney U-test. ** p<0.01 vs. C (PBS).

FIG. 98. Effect of EV of Chlorella sp. MACC-360 origin-using lyophilized (lio) and fresh sample-on peritoneal dialysis effluent (PDE) induced proliferation of primary human colon fibroblast from patient A (phCFB/A). phCFB/A cells were treated with PDE and with 3×10\hat{ }8 particles/ml of EVs of Chlorella sp. MACC-360 origin—from lyophilized (lio) and fresh sample—for 24 hours. PBS and peritoneal dialysis fluid (PDF) were used as vehicle controls. Cell proliferation was determined by MTT assay. The concentration of EVs in the figures refers to the final concentration of the stock solution of the EVs used for treatment of the cells. Results are presented as mean±SD, dots represent individual values (n=6/group). Analysis of significance was performed by unpaired t-test. ** p<0.01 vs. C (PBS), ##p<0.01 vs. C (PDF), $$ p<0.01 vs. PDE.

FIG. 99. Effect of EV of Chlorella sp. MACC-360 origin using lyophilized (lio) and fresh sample on cell death of peritoneal dialysis effluent (PDE) treated primary human colon fibroblast from patient A (phCFB/A). phCFB/A cells were treated with PDE and with 3×10\hat{ }8 particles/ml of EVs of Chlorella sp. MACC-360 origin—from lyophilized (lio) and fresh sample—for 24 hours. PBS and peritoneal dialysis fluid (PDF) were used as vehicle controls. Cytotoxicity was determined by LDH assay. The concentration of EVs in the figures refers to the final concentration of the stock solution of the EVs used for treatment of the cells. Results are presented as mean±SD, dots represent individual values (n=6/group). Analysis of significance was performed by unpaired t-test and Mann-Whitney U-test. ** p<0.01 vs. C (PBS).

FIG. 100. Effect of EV of Chlorella sp. MACC-360 origin-using fresh, hydrochloric acid (HCl) treated (37° C., 1 h, pH=4.6) and 0.25% trypsin-EDTA treated (TFF: trypsin-EDTA=2:1 ratio, 37° C., 1 h) sample-on peritoneal dialysis effluent (PDE) induced proliferation of primary human colon fibroblast from patient A (phCFB/A). phCFB/A cells were treated with PDE and with 3×10\hat{ }8 particles/ml EVs of Chlorella sp. MACC-360 origin-using fresh, HCl treated (37° C., 1 h) and trypsin treated (37° C., 1 h) sample—for 24 hours. PBS and peritoneal dialysis fluid (PDF) were used as vehicle controls. Cell proliferation was determined by MTT assay. The concentration of EVs in the figures refers to the final concentration of the stock solution of the EVs used for treatment of the cells. Results are presented as mean±SD, dots represent individual values (n=6/group). Analysis of significance was performed by unpaired t-test. ** p<0.01 vs. C (PBS), ##p<0.01, $$ p<0.01 vs. PDE.

FIG. 101. Effect of EV of Chlorella sp. MACC-360 origin, using fresh, heat denatured (denat), hydrochloric acid (HCl) treated, heat denatured and HCl treated (denat+HCl) and proteinase K treated (protK) samples on platelet derived growth factor B (PDGF-B) induced proliferation of primary human colon fibroblast from patient A (phCFB/A). phCFB/A cells were treated with PDGF-B and with 3×10\hat{ }8 particles/ml of EVs of Chlorella sp. MACC-360 origin for 24 hours. PBS was used as vehicle control. Cell proliferation was determined by MTT assay. The concentration of EVs in the figures refers to the final concentration of the stock solution of the EVs used for treatment of the cells. Results are presented as mean+SD, dots represent individual values (n=6/group). Analysis of significance was performed by unpaired t-test. ##p<0.01 vs. C (PBS), $$ p<0.01 vs. PDGF-B.

FIG. 102. Effect of EV of Chlorella sp. MACC-360 origin in combination with MACC-1 (Chlorella vulgaris) and MACC-908 (Spirulina platensis) on peritoneal dialysis effluent (PDE) induced proliferation of primary human colon fibroblast from patient A (phCFB/A). phCFB/A cells were treated with PDE and 3×10\hat{ }8 particles/ml of EVs of MACC-360, MACC-1 and MACC-908 origin or their combination for 24 hours. PBS and peritoneal dialysis fluid (PDF) were used as vehicle controls. Cell proliferation was determined by MTT assay. The concentration of EVs in the figures refers to the final concentration of the stock solution of the EVs used for treatment of the cells. Results are presented as mean±SD, dots represent individual values (n=5/group). Analysis of significance was performed by two-way ANOVA. ** p<0.01 vs. PBS, ##p<0.01 vs. PDF, $$ p<0.01 vs. PBS; PDE.

FIG. 103. Effect of EV of Chlorella sp. MACC-360 origin in combination with platelet derived growth factor receptor alpha (PDGFR-α) inhibitor Ponatinib on platelet derived growth factor B (PDGF-B) induced proliferation of primary human colon fibroblast from patient A (phCFB/A). phCFB/A cells were treated with PDGF-B and Ponatinib in the absence or presence of 3×10\hat{ }8 particles/ml of EVs of MACC-360 origin for 24 hours. PBS was used as vehicle controls. Cell proliferation was determined by MTT assay. The concentration of EVs in the figures refers to the final concentration of the stock solution of the EVs used for treatment of the cells. Results are presented as mean±SD, dots represent individual values (n=5/group). Analysis of significance was performed by one-way ANOVA. ##p<0.01 vs. C (PBS), $$ p<0.01 vs. PDGF-B, €€ p<0.01 vs. PDGF-B, 202 p<0.01 vs. PDGF-B.

FIG. 104. Effect of EV of Chlorella sp. MACC-360 origin in combination with platelet derived growth factor receptor beta (PDGFR-β) inhibitor Axitinib on platelet derived growth factor B (PDGF-B) induced proliferation of primary human colon fibroblast from patient A (phCFB/A). phCFB/A cells were treated with PDGF-B and Axitinib in the absence or presence of 3×10\hat{ }8 particles/ml EVs of MACC-360 origin for 24 hours. PBS was used as vehicle controls. Cell proliferation was determined by MTT assay. The concentration of EVs in the figures refers to the final concentration of the stock solution of the EVs used for treatment of the cells. Results are presented as mean±SD, dots represent individual values (n=5/group). Analysis of significance was performed by one-way ANOVA. ##p<0.01 vs. C (PBS), $$ p<0.01 vs. PDGF-B, €€ p<0.01 vs. PDGF-B, 202 p<0.01 vs. PDGF-B.

FIG. 105. Effect of EV of Chlorella sp. MACC-360 origin on recombinant protein mix (recMix) (−1, −2) and peritoneal dialysis effluent (PDE) (−3, −4) induced protein kinase phosphorylation in primary human colon fibroblasts from patient A (phCFB/A). Protein kinase phosphorylation was determined by Proteome Profiler Human Phospho-Kinase Array Kit. Analytes and their membrane positions on array membrane (Table FIG. 105-1). Representative images of the array membranes of various treatment groups in each experiments (−1, −2). In each experiments phCFB/A cells were treated for 30 minutes. The mean pixel density of the analytes were quantified by densitometry. In each experiments the fold change values of the optical density of the given groups were compared (FIG. 105-2, Table FIG. 105-3), or were normalized and presented as the ratio of their control values (−3, −4).

Table FIG. 105-1

Human Phospho-Kinase Array analytes and their membrane coordinates

| | 1 | 2 | 3 | 4 | 5 | 6 | 7 | 8 | 9 |
|---|---|---|---|---|---|---|---|---|---|
| A | Ref. | Ref. | | | | | | | |
| B | | | CREB S133 | CREB S133 | EGFR | EGFR | ENOS | ENOS | ERK1/2 |
| | | | | | Y1086 | Y1086 | S1177 | S1177 | T202/Y204 |
| C | | | Fgr Y412 | Fgr Y412 | GSK-3α/β | GSK-3α/β | GSK-3β | GSK-3β | Hsp27 |
| | | | | | S21/S9 | S21/S9 | S9 | S9 | S78/S82 |
| D | | | JNK1/2/3 | JNK1/2/3 | Lck Y394 | Lck Y394 | Lyn 397 | Lyn 397 | MSK1/2 |
| | | | T183/185, | T183/185, | | | | | S376/S360 |
| | | | T221/223 | T221/223 | | | | | |
| E | | | p38α | p38α | PDGFRβ | PDGFRβ | PLC-γ1 | PLC-γ1 | Src Y419 |
| | | | T180/Y182 | T180/Y182 | Y751 | Y751 | Y783 | Y783 | |
| F | | | STAT2 Y689 | STAT2 Y689 | STAT5a/b | STAT5a/b | WNK1 | WNK1 | Yes Y426 |
| | | | | | Y694/Y699 | Y694/Y699 | T60 | T60 | |
| G | Ref. | Ref. | β-Catenin | β-Catenin | | | | | PBS |

| | Human Phospho-Kinase Array analytes and their membrane coordinates | | | | | | | | |
|---|---|---|---|---|---|---|---|---|---|
| | 10 | 11 | 12 | 13 | 14 | 15 | 16 | 17 | 18 |
| A | | Akt1/2/3 | Akt1/2/3 | Akt1/2/3 | Akt1/2/3 | | | Ref. | Ref. |
| | | T308 | T308 | S473 | S473 | | | | |
| B | ERK1/2 | Chk-2 T68 | Chk-2 T68 | c-Jun S63 | c-Jun S63 | | | | |
| | T202/Y204 | | | | | | | | |
| C | Hsp27 | p53 S15 | p53 S15 | p53 S46 | p53 S46 | p53 S392 | p53 S392 | | |
| | S78/S82 | | | | | | | | |
| D | MSK1/2 | p70S6 | p70S6 | p70S6 | p70S6 | PRAS40 | PRAS40 | | |
| | S376/S360 | Kinase T389 | Kinase | Kinase | Kinase | T246 | T246 | | |
| | | | T389 | T421/S424 | T421/S424 | | | | |
| E | Src Y419 | PYK2 Y402 | PYK2 | RSK1/2 | RSK1/2 | RSK1/2/3 | RSK1/2/3 | | |
| | | | Y402 | S221/227 | S221/227 | S380/S386/ | S380/S386/ | | |
| | | | | | | S377 | S377 | | |
| F | Yes Y426 | STAT1 | STAT1 | STAT3 | STAT3 | STAT3 | STAT3 | | |
| | | Y701 | Y701 | Y705 | Y705 | S727 | S727 | | |
| G | PBS | STAT6 | STAT6 | Hsp60 | Hsp60 | | | PBS | PBS |
| | | Y641 | Y641 | | | | | | |

TABLE
FIG. 105-2

| | Fold change values of compared groups | | |
|---|---|---|---|
| Phosphorilated | Control | Control vs. | recMix vs. |
| kinases | vs. EV | recMix | recMix + EV |
| CREB (S133) | 0.83 ↓ | 0.88 ↓ | 2.59 ↑ |
| EGFR (Y1086) | 1.09 ↑ | 1.46 ↑ | 1.06 ↑ |
| ENOS (S1177) | 0.78 ↓ | 1.03 ↑ | 1.04 ↑ |
| ERK1/2 (T202/204) | 0.73 ↓ | 8.05 ↑ | 0.43 ↓ |
| AKT1/2/3 (T308) | 0.76 ↓ | 9.96 ↑ | 0.45 ↓ |
| AKT1/2/3 S473 | 0.71 ↓ | 37.97 ↑ | 0.34 ↓ |
| Chk-2 (T68) | 0.74 ↓ | 1.22 ↑ | 1.28 ↑ |
| c-Jun (S63) | 0.63 ↓ | 2.51 ↑ | 0.93 ↓ |
| Fgr (Y412) | 0.96 ↓ | 1.46 ↑ | 0.71 ↓ |
| GSK-3α/β (S21/S9) | 0.96 ↓ | 1.40 ↑ | 1 |
| GSK-3ß (S9) | 0.75 ↓ | 1.50 ↑ | 1.19 ↑ |
| HSP27 (S78/S82) | 3.08 ↓ | 5.04 ↑ | 0.63 ↓ |
| p53 (S15) | 0.79 ↓ | 1.99 ↑ | 0.57 ↓ |
| p53 (S46) | 0.95 ↓ | 1.47 ↑ | 0.9 ↓ |
| p53 (S392) | 0.62 ↓ | 1.02 ↑ | 0.94 ↓ |
| JNK1/2/3 (T183/185) | 0.95 ↓ | 1.94 ↑ | 0.58 ↓ |
| Lck (Y394) | 0.63 ↓ | 2.12 ↑ | 0.42 ↓ |
| Lyn (397) | 0.70 ↓ | 1.41 ↑ | 0.55 ↓ |
| MSK1/2 (S376/S360) | 1.03 ↑ | 4.68 ↑ | 0.52 ↓ |
| p70 S6 Kinase (T389) | 1.11 ↑ | 4.61 ↑ | 0.47 ↓ |
| p70 S6 Kinase (T421/ | 0.88 ↓ | 1.47 ↑ | 0.69 ↓ |
| S424) | | | |
| PRAS40 (T246) | 0.70 ↓ | 1.63 ↑ | 1.06 ↑ |
| p38α(T180/Y182) | 0.71 ↓ | 0.9 ↓ | 1 |
| PDGFRß (Y751) | 0.79 ↓ | 1.73 ↑ | 0.56 ↓ |
| PLC-γ1(Y783) | 0.96 ↓ | 1.12 ↑ | 0.83 ↓ |
| Src (Y419) | 0.89 ↓ | 1.22 ↑ | 1.46 ↑ |
| PYK2 (Y402) | 0.82 ↓ | 1.63 ↑ | 1.06 ↑ |
| RSK1/2 (S221/S227) | 0.83 ↓ | 1.17 ↑ | 0.72 ↓ |
| RSK1/2/3 | 1.23 ↑ | 2.58 ↑ | 0.4 ↓ |
| (S380/S386/S377) | | | |
| STAT2 (Y689) | 0.85 ↓ | 1.12 ↑ | 1.22 ↑ |
| STAT5a/b (Y694/Y699) | 0.99 ↓ | 0.92 ↓ | 0.98 ↓ |
| WNK1 (T60) | 0.78 ↓ | 1.49 ↑ | 1.33 ↑ |
| Yes (Y426) | 0.99 ↓ | 1.58 ↑ | 1.42 ↑ |
| STAT1 (Y701) | 0.79 ↓ | 1.76 ↑ | 1.11 ↑ |
| STAT3 (Y705) | 1.50 ↑ | 4.32 ↑ | 1.20 ↑ |
| STAT3 (S727) | 0.93 ↓ | 2.33 ↑ | 0.77 ↓ |
| ß-Catenin | 0.97 ↓ | 1.39 ↑ | 0.87 ↓ |
| STAT6 (Y641) | 0.73 ↓ | 0.80 ↓ | 2.13 ↑ |
| HSP60 | 0.83 ↓ | 2.99 ↑ | 0.91 ↓ |

TABLE
Figure 105-3

| | Fold change values of compared groups | | |
|---|---|---|---|
| Phosphorylated | Control | Control | PDE vs. |
| kinases | vs. EV | vs. PDE | PDE + EV |
| CREB | 1.34 ↑ | 1.5 ↑ | 1.08 ↑ |
| EGFR | 1.39 ↑ | 1.17 ↑ | 1.24 ↑ |
| ENOS | 1.12 ↑ | 1.03 ↑ | 1.17 ↑ |
| ERK1/2 | 1.18 ↑ | 1.5 ↑ | 1.09 ↑ |
| AKT1/2/3 | 0.9 ↓ | 1.34 ↑ | 1.34 ↑ |
| AKT1/2/3 | 0.75 ↓ | 1.94 ↑ | 0.84 ↓ |
| Chk-2 | 0.32 ↓ | 0.4 ↓ | 6.78 ↑ |
| c-Jun | 0.84 ↓ | 4.92 ↑ | 0.98 ↓ |
| FGR | 1.13 ↑ | 0.87 ↓ | 1.18 ↑ |
| GSK-3α/β | 0.84 ↓ | 0.79 ↓ | 1.19 ↑ |
| GSK-3ß | 0.95 ↓ | 1.06 ↑ | 1.19 ↑ |
| HSP27 | 0.82 ↓ | 0.9 ↓ | 1.18 ↑ |
| p53 | 1.51 ↑ | 1.95 ↑ | 1.83 ↑ |
| p53 | 1.23 ↑ | 1.95 ↑ | 1.04 ↑ |
| p53 | 0.96 ↓ | 3.77 ↑ | 0.7 ↓ |
| JNK1/2/3 | 1.13 ↑ | 0.93 ↓ | 1.27 ↑ |
| Lck | 0.81 ↓ | 0.77 ↓ | 1.7 ↓ |
| Lyn | 0.91 ↓ | 1.16 ↑ | 1.36 ↑ |
| MSK1/2 | 0.89 ↓ | 1.37 ↑ | 1.29 ↑ |
| p70 S6 Kinase | 1.12 ↑ | 2.2 ↑ | 1.11 ↑ |
| p70 S6 Kinase | 0.51 ↓ | 3.2 ↑ | 0.88 ↓ |
| PRAS40 | 1.14 ↑ | 1.96 ↑ | 0.93 ↓ |
| p38α | 1.09 ↑ | 0.89 ↑ | 1.23 ↑ |
| PDGFRß | 0.94 ↓ | 0.75 ↑ | 1.34 ↑ |
| PLC-γ1 | 0.89 ↓ | 1.03 ↑ | 0.88 ↓ |
| SRC | 0.73 ↓ | 0.94 ↓ | 1.2 ↑ |
| PYK2 | 0.68 ↓ | 1.06 ↑ | 1.46 ↑ |
| RSK1/2 | 0.68 ↓ | 1.3 ↑ | 0.94 ↓ |
| RSK1/2/3 | 0.73 ↓ | 1.67 ↑ | 0.78 ↓ |
| STAT2 | 1.01 ↑ | 0.92 ↓ | 1.3 ↑ |
| STAT5a/b | 0.9 ↓ | 0.78 ↓ | 1.27 ↑ |
| WNK1 | 0.8 ↓ | 1.25 ↑ | 0.92 ↓ |
| Yes | 1.13 ↑ | 1.3 ↑ | 1.22 ↑ |
| STAT1 | 0.87 ↓ | 0.6 ↓ | 4.43 ↑ |
| STAT3 | 1 | 1.85 ↑ | 4.44 ↑ |
| STAT3 | 0.78 ↓ | 2.23 ↑ | 0.61 ↓ |
| ß-Catenin | 1.1 ↑ | 0.95 ↓ | 1.36 ↑ |
| STAT6 | 0.6 ↓ | 1.2 ↑ | 1.09 ↑ |
| HSP60 | 5.22 ↑ | 2.73 ↑ | 14.25 ↑ |

FIG. 106. Effect of EV of Spirulina platensis powder origin isolated by extraction (SP extr) on peritoneal dialysis effluent (PDE) induced proliferation of primary human colon fibroblast from patient A (phCFB/A). phCFB/A cells were treated with PDE and with 18 μg EV of Spirulina platensis powder origin for 24 hours. PBS and peritoneal dialysis fluid (PDF) were used as vehicle controls. Cell proliferation was determined by MTT assay. Results are presented as mean±SD, dots represent individual values (n=6/group). Analysis of significance was performed by unpaired t-test or Mann-Whitney U-test. ##p<0.01 vs. C (PBS), $$ p<0.01 vs. PDE.

FIG. 107. Illustration of interactions among inflammation, fibrosis, and cancer in human disease. These pathologic processes cross-interact with each other at multiple levels and by multiple means, which are recognized as the causes for the development of many diseases, as well as the important host factors to influence the effectiveness of drug therapy. CAF: cancer-associated fibroblast; DAMP: danger-associated molecular pattern; ECM: extracellular matrix; IL: interleukin; PAMP: pathogen-associated molecular pattern; ROS: reactive oxygen species; TAM: tumor-associated macrophage; TAN: tumor-associated neutrophil; TGF: transforming growth factor [Dong, J., & Ma, Q. (2019). Integration of inflammation, fibrosis, and cancer induced by carbon nanotubes. Nanotoxicology, 13 (9), 1244-1274.]

### DETAILED DESCRIPTION OF THE INVENTION

It has been known in the art that extracellular vesicles (EVs) potential vectors for delivering drugs in therapeutic treatment.

The present invention relates to uses of algal EVs in therapy, wherein the EVs themselves have favourable effects for the health of the subject to whom they are administered.

Specifically, the present inventors have surprisingly found that EVs isolated from algae, including procaryotic and eucaryotic algae as defined herein, exert a complex beneficial effect in a large number of fibrosis and neoplasia as well as inflammation models, which involves inhibiting extracellular matrix (ECM) production, cell proliferation and cell migration and also increasing the production of anti-inflammatory cytokines including IL-6, IL-10, and TGFb.

These processes (i.e. ECM production, cell proliferation, cell migration and inflammation) are useful in maintaining or restoring the health of a subject (e.g. after injury or infection), however, when excessive or uncontrolled they contribute to various disorders. The processes may indicate stages of the development of various disease conditions and also play a role in the same. They are not independent and overlap as stages of the development of symptoms. Necessarily, they are orchestrated by a complex network of inter-related biochemical and signalling pathways, still, they can be thought of as details or pieces of a bigger picture. These processes, like stages of inflammation, tissue scarring or progressive fibrosis, excessive cell proliferation, migration or non-physiological ECM production and/or deposition therefore can be considered as conditions to be treated to restore or prevent impairment of the health of a subject. (FIG. 107) The etiology of a number of symptomatically defined disorders involves these (excessive or uncontrolled) processes, which should be treated themselves or in the framework of the treatment of a given disease. While specific molecules used as active agents in medicine act typically at several drugs and off target points of various biochemical pathways, typically one or two of them being considered as the main “entry point(s)”, algal EVs as active agents have, as shown by the abundant experimental evidence provided by the inventors, act at different off targets which is in harmony of their complex, still well-defined nature.

Surprisingly, these EVs have effect at several points of this matrix of conditions including, broadly speaking, inflammatory processes, excessive cell proliferation, migration and ECM production and deposition, related to, among others, inflammation, fibrosis, neoplasias as shown by exemplary model experiments taught herein. In an example EVs resulted in increased anti-inflammatory IL-10 production.

In a particular embodiment EVs effect the production of IL-6, IL-10 and/or TGFbeta having e.g. the following roles:
IL6—Inhibition of TNF and IL-1 production by macrophages
IL10—Inhibition of monocyte/macrophage and neutrophil cytokine production and inhibition of TH1-type lymphocyte responses; having humoral and cellular effect,
TGFb—Inhibition of monocyte/macrophage MHC, class II expression and proinflammatory cytokines synthesis; antiinflammatory, however, contributes to the production of proinflammatory cytokines.

Thus, typically the cytokines affected by the EVs of the invention are Janus faced molecules having anti-inflammatory aspect.

Disorders according to the invention usually induce or are associated with inflammation. For example, inflammation is interrelated with fibrosis. The immune response modulated by EVs may be multiple fold due to the cytokines modulated and finally it depends on the specific condition. It is plausible, however, that the result of the modulation of the immune response in beneficial herein. On the one hand it appears that EVs do not elicit directly and strongly proinflammatory response. This is well supported by animal experiments provided herein. For example in the DSS induced inflammation model the animals had reduced DAI score and clinically improved status. In an other experiment antifibrotic effect of EVs was also examined in vivo, in the chlorhexidine gluconate (CG) induced peritoneal fibrosis model. EVs significantly inhibited the increase of the submesothelial thickness induced by CG. Submesothelial thickness is in positive correlation with the severity of peritoneal fibrosis, thus the inflammation could not result in a progressive fibrosis (FIG. 71).

Therefore a modulation of inflammation by EVs contributes to the beneficial effect thereof.

In a further embodiment fibrosis is considered herein as a dynamic process of ECM production or degradation. An abnormal process (like injury on the one hand and progressive fibrosis on the other) shifts the equilibrium of this dynamic process to the extreme or removes from equilibrium. In a variant the ECM production/degradation becomes irregulated. In a preferred embodiment the EVs or the compositions of the invention improve the regulation of ECM production, preferably to maintain the dynamic nature thereof or maintain the normal equilibrium.

For example, in certain experiments the inventors have found that EVs shifted ECM production under the control level which may suggest a regulatory role and a shift in the ECM production/degradation dynamism.

As an example in diabetic ulcus an induction of ECM is useful.

These features of the EVs can be utilized to treat mammalian subjects having an impaired health status to be restored or use the EVs in prophylaxis. In some embodiments the EVs can be used to improve performance of the subject.

In a sense ECM-production, fibrosis, cell migration even inflammation are natural processes, which are to be kept within limits and therefore mildly alleviate them. An example is wound healing wherein fibrotic processes necessarily serve wound healing, however, under physiological conditions such processes like deposition of ECM are controlled and restore tissue architecture and lead to the restoration of the function

As another example in aging fibrosis may be considered as being necessarily present.

However, quite often, kidney is subject to progressive fibrotic processes and thereby impairment in operation in otherwise healthy elderly people.

In an aspect therefore the composition of the invention may be a part of the diet and used as a dietary supplement, nutraceutical, functional food, nutritional food, including beverages, or in certain cases as a medical food or beverages.

In other variants, if applied on the skin, cosmetic treatment may have a similar role of maintaining or arriving at a healthy condition of the skin wherein tissues scarring is not explicit any more.

Besides restoring health, treating and preventing disease conditions, algal EVs of the present invention are useful to control or regulate these processes and thereby improve performance of an otherwise healthy subject. In their experiments the present inventors have found a general anti-fibrotic effect through a very broad range of algae types wherein profibrotic inducers were applied on lung, peritoneal, colon, and skin fibroblasts and essentially every alga type tested resulted in a reduced fibroblast proliferation as shown by e.g. MTT assays. Nevertheless, ECM production apparently was increased and not reduced in case of a few alga species/strains, in particular on skin fibroblasts, whereas it was reduced in others. The data together suggest a regulatory role of alga EVs in fibroblast wherein, besides a pronounced antifibrotic effect ECM regulation is part of the general benefit. Preferred effect of combination of algal strains has also been shown by the inventors (see e.g. Figure X 102). It is plausible that by combination of alga strains the anti-fibrotic effect can be optimized and a careful selection of strains would result in a marked anti-fibrotic effect with balanced ECM-production. This may have an advantage in any anti-fibrotic (anti-fibroblast) application, in particular on lung, peritoneal, colon, and skin, fibroblast, particularly preferably on skin fibroblasts.

### Algae Useful in the Invention

Algae which can be used in the present invention include procaryotic algae and eukariotic algae e.g. as listed in the Definitions.

In particular, algae used herein are micro and macro algae.

In particular, algae used herein are prokaryotic or eukaryotic algae.

In an embodiment, eucaryotic algae as used herein are organisms that preferably belong to the following groups: Chlorophyta, Streptophyta, Rhodophyta, and Ochrophyta phylum Eucaryotic algae as used herein are organisms that preferably belong to the following groups: Chlorophyta (green algae), preferably to Ulvophyceae, Chlorophyceae, Trebouxiophyceae, Chlorodendrophyceae, Pyramimonadales, Mamiellophyceae, Pycnococcaceae, Nephroselmidaceae, Prasinococcales or Palmophyllales, more preferably to Ulvophyceae, Chlorophyceae or Trebouxiophyceae;
Streptophyta (Charophyta, green algae),
preferably to Mesostigmatophyceae, Chlorokybophyceae, Klebsormidiophyceae, Charophyceae, Zygnematophyceae, Coleochaetophyceae, more preferably to Klebsormidiophyceae or Zygnematophyceae;
   Phaeophyceae (brown algae)
   Ochrophytina (Ochrophyta)
   Rhodophyta (red algae), preferably to Cyanidiophyceae, Rhodellophyceae, Compsopogonophyceae, Bangiophyceae, Florideophyceae, Porphyridiophyceae, Stylonematophyceae, more preferably to Florideophyceae.
   Glaucophyta, preferably to Cyanophoraceae (Cyanophorales), Gloeochaetaceae (Gloeochaetales), Glaucocystidaceae (Glaucocystales).

Eucaryotic algae as used herein are organisms that may belong to the following groups:
Euglenozoa (Euglenophyceae),
stramenopiles,
Dinoflagellata,
Haptophyta (Haptophytina, Prymnesiophyta, Haptophyceae, Prymnesiophyceae),
Cryptophyta (Cryptophyceae, Cryptomonada),
Chlorarachniophyceae.

Eucaryotic algae as used herein are organisms that preferably belong to the following groups: Chlorophyta, Streptophyta (Charophyta), Ochrophyta, Rhodophyta.

Eucaryotic algae as used herein are organisms that preferably belong to the following groups: Ulvophyceae, Chlorophyceae, Trebouxiophyceae, Chlorodendrophyceae, Pyramimonadales, Mamiellophyceae, Pycnococcaceae, Nephroselmidaceae, Prasinococcales, Palmophyllales, Mesostigmatophyceae, Chlorokybophyceae, Klebsormidiophyceae, Charophyceae, Zygnematophyceae, Coleochaetophyceae, Phaeophyceae, Ochrophytina (Ochrophyta), Cyanidiophyceae, Rhodellophyceae, Compsopogonophyceae, Bangiophyceae, Florideophyceae, Porphyridiophyceae, Stylonematophyceae,

Cyanophoraceae (Cyanophorales), Gloeochaetaceae (Gloeochaetales), Glaucocystidaceae (Glaucocystales), more preferably to Ulvophyceae, Chlorophyceae, Trebouxiophyceae, Klebsormidiophyceae Zygnematophyceae, Phaeophyceae, Ochrophytina (Ochrophyta).

Eucaryotic algae as used herein are organisms that preferably belong to the following groups: Ulvophyceae, Chlorophyceae, Trebouxiophyceae, Klebsormidiophyceae, Zygnematophyceae, Phaeophyceae, Ochrophytina

### (Ochrophyta), Florideophyceae.

In a particular embodiment algae may be selected from the group consisting of the following algae species:

| | Taxonomy | | |
|---|---|---|---|
| Name | classification | Algae groups | Habitat |
| Alaria esculenta | Ochrophyta | Macroalgae | Seawater |
| Alsidium | Rhodophyta | Macroalgae | Seawater |
| helminthochorton | | | |
| Arthrospira | Cyanobacteriota | Cyanobacteria | Freshwater |
| fusiformis | | | |
| Arthrospira indica | Cyanobacteriota | Cyanobacteria | Freshwater |
| Chlorella | Chlorophyta | Microalgae | Freshwater |
| luteoviridis | | | |
| Chondrus crispus | Rhodophyta | Macroalgae | Seawater |
| Corallina officinali | Rhodophyta | Macroalgae | Seawater |
| Dunaliella salina | Chlorophyta | Microalgae | Freshwater |
| Durvillea | Ochrophyta | Macroalgae | Seawater |
| antarctica | | | |
| Ecklonia bicyclis | Ochrophyta | Macroalgae | Seawater |
| Auxenochlorella | Chlorophyta | Microalgae | Freshwater |
| protothecoides | | | |
| Eisenia bicyclis | Ochrophyta | Macroalgae | Seawater |
| Enteromorpha sp | Chlorophyta | Macroalgae | Seawater |
| Ulva intestinalis | Chlorophyta | Macroalgae | Seawater |
| Porphyra dioica | Rhodophyta | Macroalgae | Seawater |
| Eucheuma | Rhodophyta | Macroalgae | Seawater |
| denticulatum | | | |
| Eucheuma | Rhodophyta | Macroalgae | Seawater |
| horridum | | | |
| Eucheuma | Rhodophyta | Macroalgae | Seawater |
| spiciforme | | | |
| Eucheuma | Rhodophyta | Macroalgae | Seawater |
| denticulatum | | | |
| Fucus serratus | Ochrophyta | Macroalgae | Seawater |
| Fucus spiralis | Ochrophyta | Macroalgae | Seawater |
| Fucus vesiculosus | Ochrophyta | Macroalgae | Seawater |
| Gelidium amansii | Rhodophyta | Macroalgae | Seawater |
| Gelidium corneum | Rhodophyta | Macroalgae | Seawater |
| Gracilaria gracilis | Rhodophyta | Macroalgae | Seawater |
| Gracilariopsis | Rhodophyta | Macroalgae | Seawater |
| longissima | | | |
| Graesiella | Chlorophyta | Microalgae | Freshwater |
| emersonii | | | |
| Haematococcus | Chlorophyta | Microalgae | Freshwater |
| pluvialis | | | |
| Jaagichlorella | Chlorophyta | Microalgae | Freshwater |
| luteoviridis | | | |
| Himanthalia | Ochrophyta | Macroalgae | Seawater |
| elongata | | | |
| Sargassum | Ochrophyta | Macroalgae | Seawater |
| fusiforme | | | |
| Laminaria | Ochrophyta | Macroalgae | Seawater |
| cloustonii | | | |
| Laminaria | Ochrophyta | Macroalgae | Seawater |
| digitata | | | |
| Laminaria | Ochrophyta | Macroalgae | Seawater |
| hyperborea | | | |
| Saccharina | Ochrophyta | Macroalgae | Seawater |
| latissima | | | |
| Saccharina | Ochrophyta | Macroalgae | Seawater |
| japonica | | | |
| Saccharina | Ochrophyta | Macroalgae | Seawater |
| longicruris | | | |
| Laminaria | Ochrophyta | Macroalgae | Seawater |
| palmata | | | |
| Saccharina | Ochrophyta | Macroalgae | Seawater |
| latissima | | | |
| Phymatolithon | Rhodophyta | Macroalgae | Seawater |
| calcareum | | | |
| Macrocystis | Ochrophyta | Macroalgae | Seawater |
| pyrifera | | | |
| Mastocarpus | Rhodophyta | Macroalgae | Seawater |
| stellatus | | | |
| Nannochloropsis | Ochrophyta. | Microalgae | Brackish |
| oculata | | | water |
| Padina pavonica | Ochrophyta | Macroalgae | Seawater |
| Phymatolithon | Rhodophyta | Macroalgae | Seawater |
| calcareum | | | |
| Porphyra dioica | Rhodophyta | Macroalgae | Seawater |
| Porphyra | Rhodophyta | Macroalgae | Seawater |
| purpurea | | | |
| Porphyra dioica | Rhodophyta | Macroalgae | Seawater |
| Porphyra palmata | Rhodophyta | Macroalgae | Seawater |
| Neopyropia tenera | Rhodophyta | Macroalgae | Seawater |
| Porphyra | Rhodophyta | Macroalgae | Seawater |
| umbilicalis | | | |
| Neopyropia | Rhodophyta | Macroalgae | Seawater |
| leucosticta | | | |
| Pyropia tenera | Rhodophyta | Macroalgae | Seawater |
| Neopyropia | Rhodophyta | Macroalgae | Seawater |
| yezoensis | | | |
| Saccharina | Ochrophyta | Macroalgae | Seawater |
| japonica | | | |
| Saccharina | Ochrophyta | Macroalgae | Seawater |
| latissima | | | |
| Saccharina | Ochrophyta | Macroalgae | Seawater |
| longicruris | | | |
| Sargassum | Ochrophyta | Macroalgae | Seawater |
| fusiforme | | | |
| Scenedesmus | Chlorophyta | Microalgae | Freshwater |
| vacuolatus | | | |
| Schizochytrium sp. | Bigyra | Labyrinthulomycete | Seawater |
| Spirulina major | Cyanobacteriota | Cyanobacteria | Freshwater |
| Ulkenia sp. | Bigyra | Labyrinthulomycete | Seawater |
| Ulva sp | Chlorophyta | Macroalgae | Seawater |
| Ulva lactuca | Chlorophyta | Macroalgae | Seawater |
| Undaria | Ochrophyta | Macroalgae | Seawater |
| pinnatifida | | | |
| Odontella aurita | Bacillariophyta | Microalgae | Seawater |
| Ecklonia cava | Ochrophyta | Macroalgae | Seawater |
| Galdieria | Rhodophyta | Microalgae | Freshwater |
| sulphuraria | | | |
| Phaeodactylum | Bacillariophyta | Microalgae | Seawater |
| tricornutum | | | |
| Aurantiochytrium | Bigyra | Microalgae | Seawater |
| limacinum | | | |
| Haematococcus | Chlorophyta | Microalgae | Freshwater |
| lacustris | | | |
| Limnospira | Cyanobacteriota | Microalgae | Freshwater |
| fusiformis | | | |
| Asparagopsis sp | Rhodophyta | Macroalgae | Seawater |
| Schizymenia dubyi | Rhodophyta | Macroalgae | Seawater |
| Sargassum | Ochrophyta | Macroalgae | Seawater |
| muticum | | | |
| Saccorhiza | Ochrophyta | Macroalgae | Seawater |
| Saccorhiza | Ochrophyta | Macroalgae | Seawater |
| polyschides | | | |
| Porphyra linearis | Rhodophyta | Macroalgae | Seawater |
| Osmundea | Rhodophyta | Macroalgae | Seawater |
| pinnatifida | | | |
| Nemalion | Rhodophyta | Macroalgae | Seawater |
| elminthoides | | | |
| Laurencia viridis | Rhodophyta | Macroalgae | Seawater |
| Laurencia spp | Rhodophyta | Macroalgae | Seawater |
| Laminaria | Ochrophyta | Macroalgae | Seawater |
| ochroleuca | | | |
| Kallymenia | Rhodophyta | Macroalgae | Seawater |
| reniformis | | | |
| Hizikia fusiformis | Ochrophyta | Macroalgae | Seawater |
| Gigartina spp | Rhodophyta | Macroalgae | Seawater |
| Gigartina pistillata | Rhodophyta | Macroalgae | Seawater |
| Gelidium spinosum | Rhodophyta | Macroalgae | Seawater |
| Gelidium microdon | Rhodophyta | Macroalgae | Seawater |
| Codium sp | Chlorophyta | Macroalgae | Seawater |
| Chondracanthus | Rhodophyta | Macroalgae | Seawater |
| teedei | | | |
| Chondracanthus | Rhodophyta | Macroalgae | Seawater |
| acicularis | | | |

In a particular embodiment the EVs are preferably derived from Chlorophyta, preferably from Chlorellaceae, more preferably from Chlorella and/or Parachlorella and/or Spirulina, highly preferably from Chlorella sorokiniana, Parachlorella kessleri, Chlorella vulgaris, Chlorella pyrenoidosa (Auxenochlorella pyrenoidosa), Chlamydomonas reinhardtii, Hormidiospora verrucosa, Haematococcus lacustris, Tetraselmis chui In a particular embodiment the EVs are preferably derived from Streptophyta preferably from Zygnema peliosporum, Klebsormidium nitens, Spirogyra sp.

In a particular embodiment the EVs are preferably derived from Ochrophyta, preferably from Vischeria polyphem and Ascophyllum nodosum

In a particular embodiment the EVs are preferably derived from Rhodophyta, preferably from Palmaria palmata

In another embodiment the EVs are from a species belonging to Cyanobacteriota, preferably to Oscillatoriophycideae more preferably to Microcoleaceae more preferably to Arthrospira highly preferably to Spirulina (Arthrospira) platensis.

Valójában ezeket vizsgáltuk: Microcystis aeruginosa, Nostoc linckia, Spirulina platensis (Arthrospira platensis) Spirulina maxima (Limnospira maxima), Arthronema africanum, Synechococcus sp., Aphanizomenon flos-aquae In a particular embodiment, the EVs are derived from Chlorophyta, Rhodophyta, Ochrophyta and Streptophyta phylum preferably from Chlorella sorokiniana, Parachlorella kessleri, Chlorella vulgaris, Chlorella pyrenoidosa (Auxenochlorella pyrenoidosa), Chlamydomonas reinhardtii, Hormidiospora verrucosa, Haematococcus lacustris, Tetraselmis chui, Zygnema peliosporum, Klebsormidium nitens, Spirogyra sp., Vischeria polyphem, Ascophyllum nodosum, Palmaria palmata

In a particular embodiment, the EVs are derived from Cyanobacteriota phylum, preferably from Microcystis aeruginosa, Nostoc linckia, Spirulina platensis (Arthrospira platensis), Spirulina maxima (Limnospira maxima), Arthronema africanum, Synechococcus sp., Aphanizomenon flos-aquae.

In a particular embodiment the algae from which the EVs are isolated belong to any of the taxonomical groups listed in “Table of algae”, below.

The results are described herein, eg. in Tables 1-1 to 1-4 and 2-1 to 2-4.

In a particularly preferred embodiment, the alga species of the invention belongs to:

Chlorophyta (phylum), core chlorophytes (clade), preferably Trebouxiophyceae (class), preferably Chlorellales (order), preferably Chlorellaceae (family), preferably Chlorella clade, preferably Chlorella genus-Cyanobacteria/Melainabacteria group, preferably Cyanobacteria (phylum), preferably Oscillatoriophycideae (subclass) preferably, Oscillatoriales (order) preferably Microcoleaceae (family), preferably, Athrospira (genus). The present inventors have examined MACC360 in a very broad range of assays as explained herein.

Having learned its several favourable effect on disease conditions several alga strains from various species have also been studied to exemplify these effects.

Specifically, the following algal strains were examined: MACC-215 (Chlamydomonas reinhardtii), MACC-1 (Chlorella vulgaris), MACC-3 (Chlorella pyrenoidosa), MACC-1023 (Hormidiospora verrucosa), MACC-21 (Zygnema peliosporum), MACC-1042 (Klebsormidium nitens), MACC-918 (Spirogyra sp)., MACC-1022 (Vischeria polyphem), MACC-888 (Microcystis aeruginosa), MACC-612 (Nostoc linckia), MACC-908 (Spirulina platensis), MACC-909 (Spirulina maxima), MACC-90 (Haematococcus lacustris), MACC-42 (Arthronema africanum), MACC-115 (Synechococcus sp), Aschophyllum nodosum, Palmaria palmata, Tetraselmis chui, Aphanizomenon flos-aquae.

### Alga Isolation

Algal EVs may be isolated in several ways according to the present invention.

To give a few examples, algae may be isolated
from supernatant of a culture of the alga cells, by isolating EVs from the culture supernatant,
from dried alga cells by rehydration of said alga cells with a medium, removing, in particular pelleting the cells and cell debris and isolating EVs from the supernatant medium,
from suspension of alga cells, in particular of cultured alga cells or dried and rehydrated alga cells, in particular rehydrated pelleted alga cells, preferably applying by a mechanical or physical effect.

In a particular embodiment algae can be isolated from alga cell suspension or from dried and later rehydrated alga cells by rupture of the alga cells (in particular by mechanic disruption or sonication).

In an embodiment the method comprises pelleting the cells and cell debris and isolating EVs from the pellet In a method cells and cell debris are pelleted from culture, or dried algae after rehydration, and the pellet is mechanically treated, e.g. extruded to produce artificial EVs.

The invention preferably relates to the method of the preparation of the algal EVs or compositions comprising said EVs, in particular for use in any of the indications definer herein, in particular, wherein upon obtaining the EVs
the algal cells are pelleted,
the pellet supernatant is filtered, preferably
cells are filtered out
an EV fraction is prepared by filtering wherein biomolecules are allowed to be transferred into the filtrate,
the EV fraction is subjected to SEC.

### Cell Proliferation and Migration in Various Disorders

Cell proliferation and migration plays a very important role in the disease conditions which are contemplated (considered) in the present invention and which are indicated herein, which is in accordance with the literature in the field as proliferation/migration disorders.

The role of proliferation and migration is to increase number of cells at a site of the body.

To see an example of proliferation/migration disorders, fibroproliferative disorders include tissue scarring as a response to chronic damage to mammalian tissues and organs. Moreover, in tissue damage typically inflammation occurs. Tissue damage increase the synthesis of inflammatory mediators, activates tissue fibroblasts, increases their divisional and migrational activity as well as the production of the extracellular matrix (ECM) components.

Fibroblasts form a rather heterogeneous group of cells in terms of their molecular markers and/or function, and probably are derived from specialized cells like mesenchymal and mesenchymal stromal cells (MSCs), endothelial, epithelial, adipose, bone marrow cells, and pericytes of mesodermal origin (Zeisberg M, Neilson E G. Biomarkers for epithelial-mesenchymal transitions. J Clin Invest. 2009 June; 119 (6): 1429-37. doi: 10.1172/JCI36183. Epub 2009 June 1. PMID: 19487819; PMCID: PMC2689132; Kalluri R. The biology and function of fibroblasts in cancer. Nat Rev Cancer. 2016 Aug. 23; 16 (9): 582-98. doi: 10.1038/nrc.2016.73. PMID: 27550820; LeBleu VS, Kalluri R. A peek into cancer-associated fibroblasts: origins, functions and translational impact. Dis Model Mech. 2018 Apr. 19; 11 (4): dmm029447. doi: 10.1242/dmm.029447. PMID: 29686035; PMCID: PMC5963854. 2018; 11: dmm029447; Lynch M D, Watt F M. Fibroblast heterogeneity: implications for human disease. J Clin January 2; 128 (1): 26-35. doi: 10.1172/JCI93555. Epub 2018 Jan. 2. PMID: 29293096; Invest. 2018 PMCID: PMC5749540). There are a number of hypotheses regarding the origin of fibroblasts, having in common the assumption that fibroblasts in different tissues are all formed by the differentiation and dedifferentiation of cells of mesodermal origin.

Fibroblasts are characterized by their intense proliferation, migration, and increased production of ECM during activation, which provided a particular example for inter-relation of these three processes. Moreover, fibrosis and chronic inflammation go hand in hand. Cytokines, growth factors, etc. produced during the inflammatory response activate fibroblasts i.e. increase their proliferation, migration and the production of the ECM.

Different PDGF isoforms, including PDGF-BB and also other factors such as TGFβ or EGF play a significant role in the activation of fibroblasts, such as their proliferation, migration, and the production of the ECM.

These growth factors and the fibroblasts also play a role in the development of different tumours and metastasis of them. The pathophysiological roles of cancer-associated fibroblasts (CAFs) in the heterogeneous tumour microenvironment have attracted increasing interest. CAFs play crucial roles in tumour progression and the response to chemotherapy. Several cytokines and chemokines are involved in the activation of CAFs, and some of these form a feedback loop between cancer cells and CAFs. In addition, the physical force between tumour cells and CAFs promotes cooperative invasion or co-migration of both types of cells (Go J. Yoshida Regulation of heterogeneous cancer-associated fibroblasts: the molecular pathology of activated signalling pathways Journal of Experimental & Clinical Cancer Research volume 39, Article number: 112 (2020)).

Proliferation, migration and ECM production of different fibroblasts, including MRC5 and primary fibroblast originated from human skin, colon or peritoneum were induced in our study by peritoneal dialysis (PD) effluent (PDE) in vitro. Moreover, proliferation, migration or ECM production of skin, colon and peritoneal primary fibroblasts was induced by PDGF-B, EGF or TGF-β treatment, respectively.

During PD the amount of glucose, AGEs, inflammatory factors, profibrotic growth factors are increased in the abdominal cavity, which all are capable to induce peritoneal thickening characterised by increased fibroblast activation and excessive deposition of ECM. Therefore PDE, which is released from the abdominal cavity at the end of dialysis contains high amount of these proinflammatory and fibrotic factors. Indeed, the present inventors have shown that the peritoneal dialysis effluent used contained CTGF (connective tissue growth factor), PDGF-B, and TGF-B, all implicated in the initiation of fibrosis (Lipson, K. E., Wong, C., Teng, Y. et al. CTGF is a central mediator of tissue remodelling and fibrosis and its inhibition can reverse the process of fibrosis. Fibrogenesis Tissue Repair 5, S24 (2012); Ying H Z, Chen Q, Zhang W Y, et al. PDGF signalling pathway in hepatic fibrosis pathogenesis and therapeutics (Review). Mol Med Rep. 2017; 16 (6): 7879-7889. doi: 10.3892/mmr.2017.7641; Gallini R, Lindblom P, Bondjers C, Betsholtz C, Andrae J. PDGF-A and PDGF-B induces cardiac fibrosis in transgenic mice. Exp Cell Res. 2016 Dec. 10; 349 (2): 282-290; Piotr Czochra, Borut Klopcic, Erik Meyer, Johannes Herkel, Jose Francisco Garcia-Lazaro, Florian Thieringer, Peter Schirmacher, Stefan Biesterfeld, Peter R. Galle, Ansgar W. Lohse, Stephan Kanzler, Liver fibrosis induced by hepatic overexpression of PDGF-B in transgenic mice, Journal of Hepatology, Volume 45, Issue 3, 2006, Pages 419-428; Meng, Xm., Nikolic-Paterson, D. & Lan, H. TGF-B: the master regulator of fibrosis. Nat Rev Nephrol 12, 325-338 (2016). Biernacka A, Dobaczewski M, Frangogiannis N G. TGF-β signalling in fibrosis. Growth Factors. 2011; 29 (5): 196-202. doi: 10.3109/08977194.2011.595714; Dirk Pohlers, Julia Brenmoehl, Ivonne Löffler, Cornelia K. Müller, Carola Leipner, Stefan Schultze-Mosgau, Andreas Stallmach, Raimund W. Kinne, Gunter Wolf, TGF-β and fibrosis in different organs—molecular pathway imprints, Biochimica et Biophysica Acta (BBA)—Molecular Basis of Disease, Volume 1792, Issue 8, 2009, Pages 746-756).

Therefore, the present inventors used PDE (see below) to mimic the complex molecular milieu of the fibrotic tissues/organs ex vivo (Yu, F., Chen, J., Wang, X. et al. Establishment of a novel mouse peritoneal dialysis-associated peritoneal injury model. Clin Exp Nephrol (2022). PMID: 35353282). This ex vivo model, being a more complex fluid, provides an environment very near to an in vivo setting.

Effect of EVs of different algal origins on peritoneal dialysis effluent (PDE) induced proliferation, cytotoxicity, collagen production and migration of primary human colon fibroblast from a patient, patient A; (phCFB/A), primary human skin fibroblast from patient A (phSFB/A), human hepatocellular carcinoma cell line (HEPG2) and human colon carcinoma cell line (Caco-2). The results are shown in Tables 1-1 to 1-5 and Tables 2-1 to 2-5. All cell types were treated with PDE and with 3×10\hat{ }8 particles EVs of the different algal origins for 24 hours. PBS and peritoneal dialysis fluid (PDF) were used as vehicle controls. Cell proliferation was determined by MTT assay, cytotoxicity was determined by LDH assay, collagen accumulation was determined by Sirius Red assay and migration was determined by TAS assay. Analysis of significance was performed by unpaired t test, Mann-Whitney test or two-way ANOVA. p<0.05 1 means that the proliferation, cytotoxicity, collagen production or migration was enhanced in the EV, PDE, or PDE+EV treated groups compared to their proper controls (control or PDE treated groups, respectively), p<0.05 | means that the proliferation, cytotoxicity, collagen production or migration was decreased in the EV, PDE, or PDE+EV treated groups compared to their proper controls (control or PDE treated groups, respectively), p=NS means that the proliferation, cytotoxicity, collagen production or migration was not significantly different in the EV, PDE, or PDE+EV treated groups compared to their proper controls (control or PDE treated groups, respectively).

A brief information of the algae strains examined is provided herein in the Table of algae:

Table of algae

| | | Taxonomy | | |
|---|---|---|---|---|
| Name | Identifier | classification | Algae groups | Habitat |
| Chlorella sorokiniana (edible) | MACC-360 | Chlorophyta | Microalgae | Freshwater |
| Parachlorella kessleri (edible) | MACC-38 | Chlorophyta | Microalgae | Freshwater |
| Chlorella vulgaris (edible) | MACC-1 | Chlorophyta | Microalgae | Freshwater |
| Chlorella pyrenoidosa | MACC-3 | Chlorophyta | Microalgae | Freshwater |
| (Auxenochlorella pyrenoidosa) | | | | |
| (edible) | | | | |
| Chlamydomonas reinhardtii | MACC-215 | Chlorophyta | Microalgae | Freshwater |
| Hormidiospora verrucosa | MACC-1023 | Chlorophyta | Microalgae | Freshwater |
| Haematococcus lacustris (edible) | MACC-90 | Chlorophyta | Microalgae | Freshwater |
| Tetraselmis chui (edible) | | Chlorophyta | Microalgae | Seawater |
| Zygnema peliosporum | MACC-21 | Streptophyta | Microalgae | Freshwater |
| Klebsormidium nitens | MACC-1042 | Streptophyta | Microalgae | Freshwater |
| Spirogyra sp. | MACC-918 | Streptophyta | Microalgae | Freshwater |
| Vischeria polyphem | MACC-1022 | Ochrophyta | Microalgae | Freshwater |
| Ascophyllum nodosum (edible) | | Ochrophyta | Macroalgae | Seawater |
| Palmaria palmata (edible) | | Rhodophyta | Macroalgae | Seawater |
| Microcystis aeruginosa | MACC-888 | Cyanobacteriota | Cyanobacteria | Freshwater |
| Nostoc linckia | MACC-612 | Cyanobacteriota | Cyanobacteria | Freshwater |
| Spirulina platensis (Arthrospira | MACC-908 | Cyanobacteriota | Cyanobacteria | Freshwater |
| platensis) | | | | |
| Spirulina maxima (Limnospira | MACC-909 | Cyanobacteriota | Cyanobacteria | Freshwater |
| maxima) | | | | |
| Arthronema africanum | MACC-42 | Cyanobacteriota | Cyanobacteria | Freshwater |
| Synechococcus sp. | MACC-115 | Cyanobacteriota | Cyanobacteria | Freshwater |
| Aphanizomenon flos-aquae | | Cyanobacteriota | Cyanobacteria | Freshwater |
| (edible) | | | | |

Results with algae strains examined herein is provided in Tables 1-1 to 1-4 and 2-1 to 2-4:

TABLE 1-1

| | | | MACC-215 | MACC-1 | MACC-3 | MACC-1023 | MACC-21 |
|---|---|---|---|---|---|---|---|
| TREATMENTS | | Compared groups | EV | EV | EV | EV | EV |
| CELL/ASSAYS | colon FB/ | control vs. EV | p < 0.05 ↓ | p < 0.05 ↓ | p < 0.05 ↓ | p < 0.05 ↓ | p < 0.05 ↓ |
| | MTT | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | p < 0.05 ↓ | p < 0.05 ↓ | p < 0.05 ↓ |
| | colon FB/ | control vs. EV | p < 0.05 ↓ | NS. | NS. | p < 0.05 ↓ | NS. |
| | SR | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↑ | NS. | p < 0.05 ↓ | NS. |
| | colon FB/ | control vs. EV | NS. | NS. | p < 0.05 ↓ | NS. | p < 0.05 ↓ |
| | TAS | control vs. PDE | p < 0.05 ↑ | p < 0.05 ↑ | p < 0.05 ↑ | p < 0.05 ↑ | p < 0.05 ↑ |
| | | PDE vs. PDE + EV | NS. | p < 0.05 ↓ | NS. | NS. | p < 0.05 ↓ |
| ANTIFIBROTIC EFFECT - colon FB | | | YES | YES | YES | YES | YES |
| WOUND HEALING EFECT OF Evs | | | | YES | | | |
| CELL/ASSAYS | skin FB/ | control vs. EV | p < 0.05 ↓ | NS. | NS. | p < 0.05 ↓ | NS. |
| | MTT | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | p < 0.05 ↓ | p < 0.05 ↓ | NS. |
| | skin FB/ | control vs. EV | NS. | NS. | p < 0.05 ↓ | NS. | NS. |
| | LDH | control vs. PDE | NS. | NS. | NS. | NS. | NS. |
| | | PDE vs. PDE + EV | NS. | NS. | NS. | NS. | NS. |
| | skin FB/ | control vs. EV | NS. | p < 0.05 ↓ | p < 0.05 ↓ | p < 0.05 ↓ | NS. |
| | SR | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | NS. | NS. | NS. |
| | skin FB/ | control vs. EV | NS. | NS. | NS. | NS. | NS. |
| | TAS | control vs. PDE | p < 0.05 ↑ | p < 0.05 ↑ | p < 0.05 ↑ | p < 0.05 ↑ | p < 0.05 ↑ |
| | | PDE vs. PDE + EV | NS. | p < 0.05 ↓ | NS. | NS. | NS. |

| | | | | | |
|---|---|---|---|---|---|
| ANTIFIBROTIC EFFECT - skin FB | YES | YES | YES | YES | YES |
| WOUND HEALING EFECT | | | | | |

TABLE 1-2

| | | | MACC-1042 | MACC-918 | MACC-1022 | MACC-888 | MACC-612 |
|---|---|---|---|---|---|---|---|
| TREATMENTS | | Compared groups | EV | EV | EV | EV | EV |
| CELL/ASSAYS | colon FB/ | control vs. EV | p < 0.05 ↓ | p < 0.05 ↓ | NS. | p < 0.05 ↓ | NS. |
| | MTT | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | p < 0.05 ↓ | p < 0.05 ↓ | p < 0.05 ↓ |
| | colon FB/ | control vs. EV | NS. | p < 0.05 ↓ | NS. | p < 0.05 ↓ | NS. |
| | SR | control vs. PDE | p < 0.05 ↑ | p < 0.05 ↑ | p < 0.05 ↑ | p < 0.05 ↑ | p < 0.05 ↑ |
| | | PDE vs. PDE + EV | NS. | p < 0.05 ↓ | NS. | NS. | p < 0.05 ↓ |
| | colon FB/ | control vs. EV | NS. | p < 0.05 ↓ | no data | no data | p < 0.05 ↓ |
| | TAS | control vs. PDE | p < 0.05 ↑ | p < 0.05 ↑ | no data | no data | p < 0.05 ↑ |
| | | PDE vs. PDE + EV | NS. | p < 0.05 ↓ | no data | no data | p < 0.05 ↓ |
| ANTIFIBROTIC EFFECT - colon FB | | | YES | YES | YES | YES | YES |
| WOUND HEALING EFECT OF Evs | | | | | | | |
| [symbol] | skin FB/ | control vs. EV | p < 0.05 ↓ | p < 0.05 ↓ | NS. | p < 0.05 ↓ | NS. |
| | MTT | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | p < 0.05 ↓ | p < 0.05 ↓ | p < 0.05 ↓ |
| | skin FB/ | control vs. EV | p < 0.05 ↑ | NS. | p < 0.05 ↑ | NS. | NS. |
| | LDH | control vs. PDE | p < 0.05 ↓ | NS. | p < 0.05 ↓ | NS. | p < 0.05 ↓ |
| | | PDE vs. PDE + EV | NS. | NS. | NS. | NS. | NS. |
| | skin FB/ | control vs. EV | NS. | p < 0.05 ↑ | NS. | NS. | p < 0.05 ↑ |
| | SR | control vs. PDE | p < 0.05 ↑ | p < 0.05 ↑ | p < 0.05 ↑ | p < 0.05 ↑ | p < 0.05 ↑ |
| | | PDE vs. PDE + EV | NS. | p < 0.05 ↑ | NS. | p < 0.05 ↓ | NS. |
| | skin FB/ | control vs. EV | NS. | NS. | p < 0.05 ↓ | no data | NS. |
| | TAS | control vs. PDE | p < 0.05 ↑ | p < 0.05 ↑ | p < 0.05 ↑ | no data | p < 0.05 ↑ |
| | | PDE vs. PDE + EV | NS. | NS. | NS. | no data | NS. |

| | | | | | |
|---|---|---|---|---|---|
| ANTIFIBROTIC EFFECT - skin FB | YES | YES | YES | YES | YES |
| WOUND HEALING EFECT | | YES | | | |

[symbol]

indicates data missing or illegible when filed

TABLE 1-3

| | | | MACC-908 | MACC-909 | MACC-90 | MACC-42 | MACC-115 |
|---|---|---|---|---|---|---|---|
| TREATMENTS | | Compared groups | EV | EV | EV | EV | EV |
| CELL/ASSAYS | colon FB/ | control vs. EV | p < 0.05 ↓ | p < 0.05 ↓ | p < 0.05 ↓ | p < 0.05 ↓ | p < 0.05 ↓ |
| | MTT | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | p < 0.05 ↓ | p < 0.05 ↓ | NS. |
| | colon FB/ | control vs. EV | p < 0.05 ↓ | NS. | NS. | p < 0.05 ↓ | NS. |
| | SR | control vs. PDE | p < 0.05 ↑ | p < 0.05 ↑ | p < 0.05 ↑ | p < 0.05 ↑ | p < 0.05 ↑ |
| | | PDE vs. PDE + EV | NS. | p < 0.05 ↑ | p < 0.05 ↓ | p < 0.05 ↓ | p < 0.05 ↓ |
| | colon FB/ | control vs. EV | no data | NS. | no data | no data | no data |
| | TAS | control vs. PDE | no data | p < 0.05 ↑ | no data | no data | no data |
| | | PDE vs. PDE + EV | no data | p < 0.05 ↓ | no data | no data | no data |
| ANTIFIBROTIC EFFECT - colon FB | | | YES | YES | YES | YES | YES |
| WOUND HEALING EFECT | | | | YES | | | |
| CELL/ASSAYS | skin FB/ | control vs. EV | p < 0.05 ↓ | p < 0.05 ↓ | p < 0.05 ↓ | p < 0.05 ↓ | NS. |
| | MTT | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | NS. | NS. | NS. |
| | skin FB/ | control vs. EV | NS. | NS. | NS. | NS. | NS. |
| | LDH | control vs. PDE | NS. | p < 0.05 ↑ | NS. | NS. | p < 0.05 ↑ |
| | | PDE vs. PDE + EV | NS. | NS. | NS. | NS. | NS. |
| | skin FB/ | control vs. EV | p < 0.05 ↓ | NS. | p < 0.05 ↑ | p < 0.05 ↑ | NS. |
| | SR | control vs. PDE | p < 0.05 ↑ | p < 0.05 ↑ | p < 0.05 ↑ | p < 0.05 ↑ | p < 0.05 ↑ |
| | | PDE vs. PDE + EV | NS. | p < 0.05 ↑ | p < 0.05 ↑ | p < 0.05 ↑ | NS. |
| | skin FB/ | control vs. EV | no data | NS. | no data | no data | no data |
| | TAS | control vs. PDE | no data | p < 0.05 ↑ | no data | no data | no data |
| | | PDE vs. PDE + EV | no data | p < 0.05 ↓ | no data | no data | no data |

| | | | | | |
|---|---|---|---|---|---|
| ANTIFIBROTIC EFFECT - skin FB | YES | YES | YES | YES | YES |
| WOUND HEALING EFECT | | YES | YES | YES | |

TABLE 1-4

| | | | Aschophyllum | Palmaria | Tetraselmis | Aphanizomenon |
|---|---|---|---|---|---|---|
| TREATMENTS | | Compared groups | nodosum EV | palmata EV | chui EV | flos-aquae EV |
| CELL/ASSAYS | colon FB/ | control vs. EV | p < 0.05 ↓ | NS. | p < 0.05 ↑ | p < 0.05 ↑ |
| | MTT | control vs. PDE | p < 0.05 ↑ | p < 0.05 ↑ | p < 0.05 ↑ | p < 0.05 ↑ |
| | | PDE vs. PDE + EV | p < 0.05 ↓ | NS. | p < 0.05 | p < 0.05 ↓ |
| | colon FB/ | control vs. EV | no data | no data | no data | no data |
| | SR | control vs. PDE | no data | no data | no data | no data |
| | | PDE vs. PDE + EV | no data | no data | no data | no data |
| | colon FB/ | control vs. EV | p < 0.05 ↓ | p < 0.05 ↓ | no data | p < 0.05 ↓ |
| | TAS | control vs. PDE | p < 0.05 ↑ | p < 0.05 ↑ | no data | p < 0.05 ↑ |
| | | PDE vs. PDE + EV | p < 0.05 ↓ | p < 0.05 ↓ | no data | p < 0.05 ↓ |
| ANTIFIBROTIC EFFECT - colon FB | | | YES | YES | YES | YES |
| WOUND HEALING EFECT OF Evs | | | | | | |
| CELL/ASSAYS | skin FB/ | control vs. EV | NS. | NS. | no data | no data |
| | MTT | control vs. PDE | p < 0.05 ↑ | p < 0.05 ↑ | no data | no data |
| | | PDE vs. PDE + EV | NS. | NS. | no data | no data |
| | skin FB/ | control vs. EV | p < 0.05 ↑ | p < 0.05 ↑ | no data | no data |
| | LDH | control vs. PDE | NS. | NS. | no data | no data |
| | | PDE vs. PDE + EV | NS. | NS. | no data | no data |
| | skin FB/ | control vs. EV | NS. | no data | no data | no data |
| | SR | control vs. PDE | p < 0.05 ↑ | no data | no data | no data |
| | | PDE vs. PDE + EV | NS. | no data | no data | no data |
| | skin FB/ | control vs. EV | no data | no data | no data | no data |
| | TAS | control vs. PDE | no data | no data | no data | no data |
| | | PDE vs. PDE + EV | no data | no data | no data | no data |

| | | | | |
|---|---|---|---|---|
| ANTIFIBROTIC EFFECT - skin FB | YES | YES | no data | no data |
| WOUND HEALING EFECT | | | | |

Cancer

TABLE 2-1

| | | Compared | MACC-215 | MACC-1 | MACC-3 | MACC-1023 | MACC-21 |
|---|---|---|---|---|---|---|---|
| TREATMENTS | | groups | EV | EV | EV | EV | EV |
| CELL/ASSAYS | HepG2/ | control vs. EV | p < 0.05 ↓ | p < 0.05 ↓ | p < 0.05 ↓ | no data | p < 0.05 ↓ |
| | MTT | control vs. PDE | p < 0.05 ↑ | p < 0.05 ↑ | p < 0.05 ↑ | no data | p < 0.05 ↑ |
| | | PDE vs. PDE + EV | NS. | p < 0.05 ↓ | p < 0.05 ↓ | no data | p < 0.05 ↓ |
| | HepG2/ | control vs. EV | p < 0.05 ↓ | p < 0.05 ↓ | NS. | no data | NS. |
| | LDH | control vs. PDE | NS. | NS. | NS. | no data | NS. |
| | | PDE vs. PDE + EV | p < 0.05 ↓ | p < 0.05 ↓ | NS. | no data | NS. |
| CELL/ASSAYS | Caco-2/ | control vs. EV | no data | no data | no data | p < 0.05 ↓ | no data |
| | MTT | control vs. PDE | no data | no data | no data | p < 0.05 ↑ | no data |
| | | PDE vs. PDE + EV | no data | no data | no data | p < 0.05 ↓ | no data |
| | Caco-2/ | control vs. EV | no data | no data | no data | NS. | no data |
| | LDH | control vs. PDE | no data | no data | no data | p < 0.05 ↑ | no data |
| | | PDE vs. PDE + EV | no data | no data | no data | p < 0.05 ↑ | no data |

| | | | | | |
|---|---|---|---|---|---|
| ANTI TUMOR EFFECT | — | YES | YES | YES | YES |

TABLE 2-2

| | | | MACC-1042 | MACC-918 | MACC-1022 | MACC-888 | MACC-612 |
|---|---|---|---|---|---|---|---|
| TREATMENTS | | Compared groups | EV | EV | EV | EV | EV |
| CELL/ASSAYS | HepG2/ | control vs. EV | p < 0.05 ↓ | NS. | NS. | no data | p < 0.05 ↓ |
| | MTT | control vs. PDE | p < 0.05 ↑ | p < 0.05 ↑ | p < 0.05 ↑ | no data | p < 0.05 ↑ |
| | | PDE vs. PDE + EV | p < 0.05 ↓ | p < 0.05 ↓ | NS. | no data | p < 0.05 ↓ |
| | HepG2/ | control vs. EV | NS. | NS. | p < 0.05 | no data | NS. |
| | LDH | control vs. PDE | NS. | NS. | NS. | no data | NS. |
| | | PDE vs. PDE + EV | p < 0.05 ↓ | NS. | p < 0.05 ↓ | no data | NS. |
| CELL/ASSAYS | Caco-2/ | control vs. EV | no data | no data | NS. | no data | no data |
| | MTT | control vs. PDE | no data | no data | p < 0.05 ↑ | no data | no data |
| | | PDE vs. PDE + EV | no data | no data | p < 0.05 ↓ | no data | no data |
| | Caco-2/ | control vs. EV | no data | no data | NS. | no data | no data |
| | LDH | control vs. PDE | no data | no data | p < 0.05 ↑ | no data | no data |
| | | PDE vs. PDE + EV | no data | no data | NS. | no data | no data |

| | | | | | |
|---|---|---|---|---|---|
| ANTI TUMOR EFFECT | YES | YES | YES | no data | YES |

TABLE 2-3

| | | | MACC-908 | MACC-909 | MACC-90 | MACC-42 | MACC-115 |
|---|---|---|---|---|---|---|---|
| TREATMENTS | | Compared groups | EV | EV | EV | EV | EV |
| CELL/ASSAYS | HepG2/ | control vs. EV | p < 0.05 ↓ | p < 0.05 ↓ | p < 0.05 ↓ | p < 0.05 ↓ | NS. |
| | MTT | control vs. PDE | p < 0.05 ↑ | p < 0.05 ↑ | p < 0.05 ↑ | p < 0.05 ↑ | p < 0.05 ↑ |
| | | PDE vs. PDE + EV | p < 0.05 ↓ | p < 0.05 ↓ | NS. | p < 0.05 ↓ | p < 0.05 ↓ |
| | HepG2/ | control vs. EV | p < 0.05 ↓ | NS. | p < 0.05 ↓ | NS. | p < 0.05 ↓ |
| | LDH | control vs. PDE | NS. | NS. | NS. | NS. | p < 0.05 ↑ |
| | | PDE vs. PDE + EV | NS. | p < 0.05 ↓ | p < 0.05 ↓ | NS. | NS. |
| CELL/ASSAYS | Caco-2/ | control vs. EV | no data | no data | no data | NS. | no data |
| | MTT | control vs. PDE | no data | no data | no data | p < 0.05 ↑ | no data |
| | | PDE vs. PDE + EV | no data | no data | no data | p < 0.05 ↓ | no data |
| | Caco-2/ | control vs. EV | no data | no data | no data | p < 0.05 ↑ | no data |
| | LDH | control vs. PDE | no data | no data | no data | p < 0.05 ↑ | no data |
| | | PDE vs. PDE + EV | no data | no data | no data | NS. | no data |

| | | | | | |
|---|---|---|---|---|---|
| ANTI TUMOR EFFECT | YES | YES | — | YES | YES |

TABLE 2-4

| | | | Aschophyllum | Palmaria | Tetraselmis | Aphanizomenon |
|---|---|---|---|---|---|---|
| TREATMENTS | | Compared groups | nodosum EV | palmata EV | chui EV | flos-aquae EV |
| CELL/ASSAYS | HepG2/ | control vs. EV | no data | no data | p < 0.05 ↓ | NS. |
| | MTT | control vs. PDE | no data | no data | p < 0.05 ↑ | p < 0.05 ↑ |
| | | PDE vs. PDE + EV | no data | no data | p < 0.05 ↓ | p < 0.05 ↓ |
| | HepG2/ | control vs. EV | no data | no data | p < 0.05 ↓ | NS. |
| | LDH | control vs. PDE | no data | no data | p < 0.05 ↑ | p < 0.05 ↑ |
| | | PDE vs. PDE + EV | no data | no data | NS. | NS. |
| [symbol] | Caco-2/ | control vs. EV | no data | no data | no data | no data |
| | MTT | control vs. PDE | no data | no data | no data | no data |
| | | PDE vs. PDE + EV | no data | no data | no data | no data |
| | Caco-2/ | control vs. EV | no data | no data | no data | no data |
| | LDH | control vs. PDE | no data | no data | no data | no data |
| | | PDE vs. PDE + EV | no data | no data | no data | no data |

| | | | | |
|---|---|---|---|---|
| ANTI TUMOR EFFECT | no data | no data | YES | YES |

[symbol]

indicates data missing or illegible when filed

The mechanism of fibrosis is actually the same or very similar in any tissue or organ. Thus, taken together, experiments with multiple types of fibroblasts treated with profibrotic growth factors, or PDE and the in vivo model of peritoneal fibrosis support that algal EVs are useful in the treatment of fibrosis or of fibroproliferative disease in practically any tissue or organ of the subject.

Below, supportive experiments and specific embodiments of the invention are discussed.

### EVs are Capable of Penetrating (Mammalian) Cells

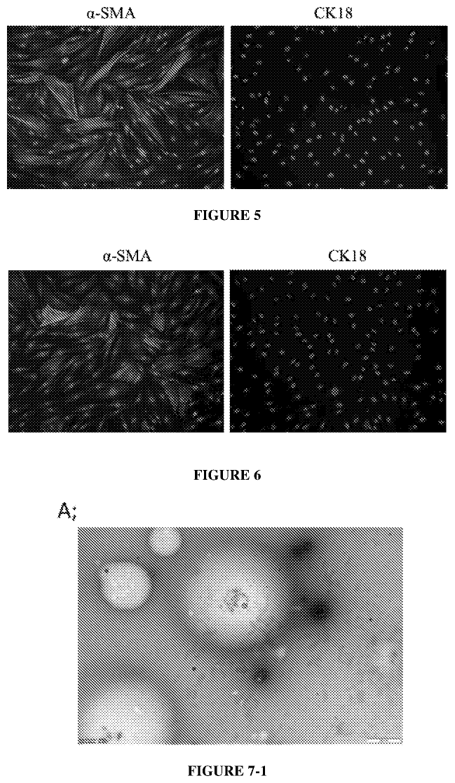

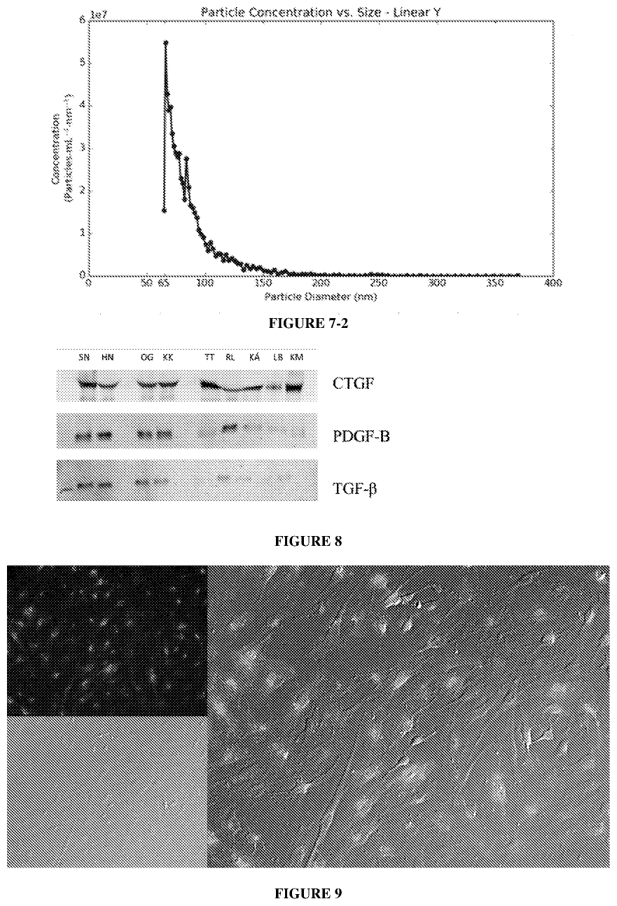

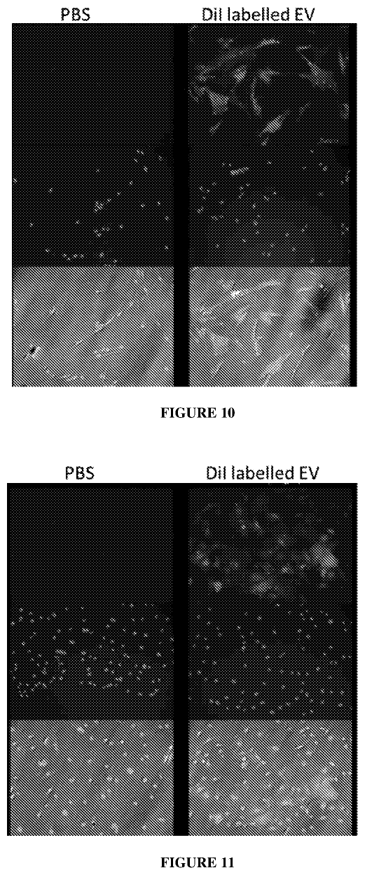

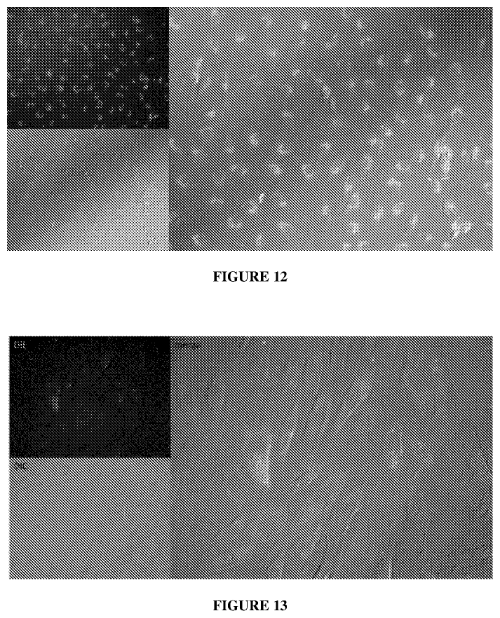

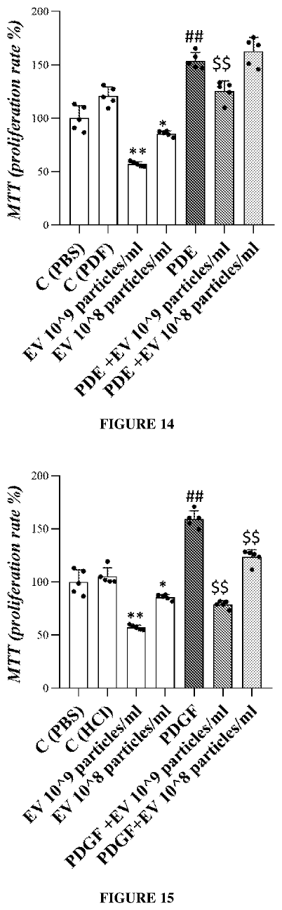

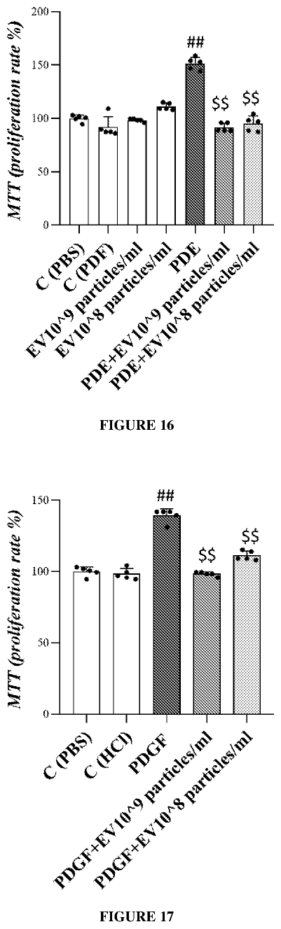

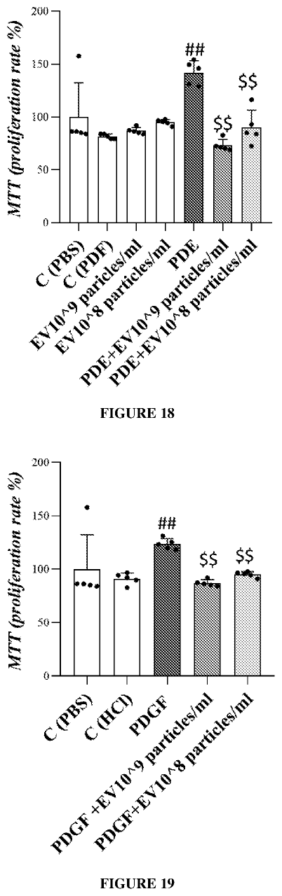

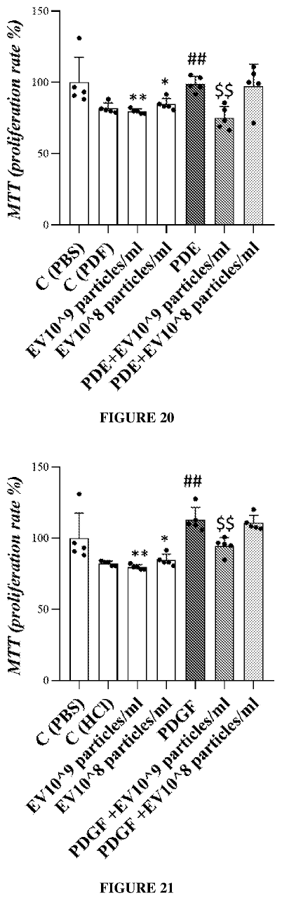

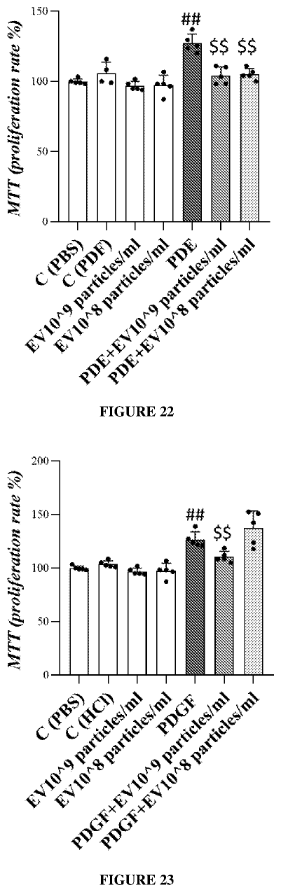

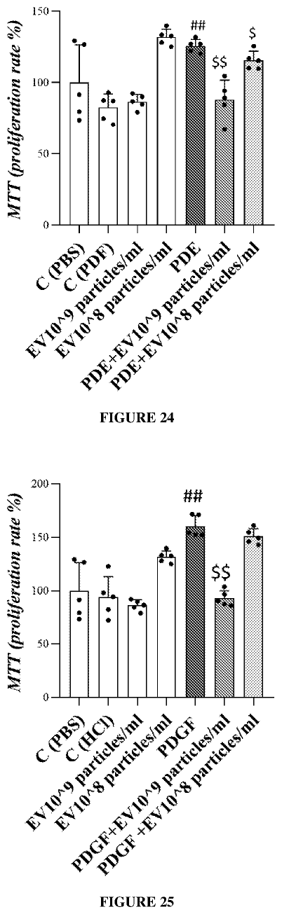

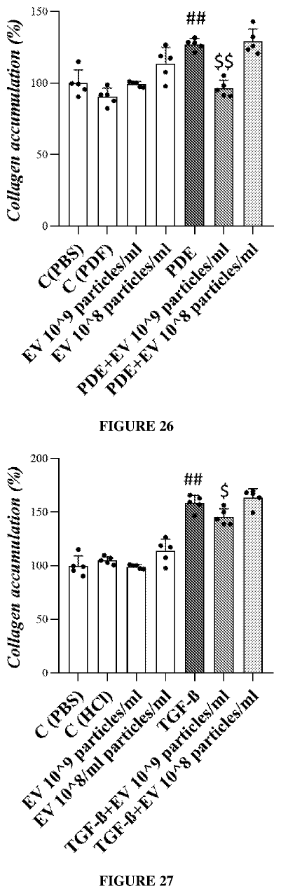

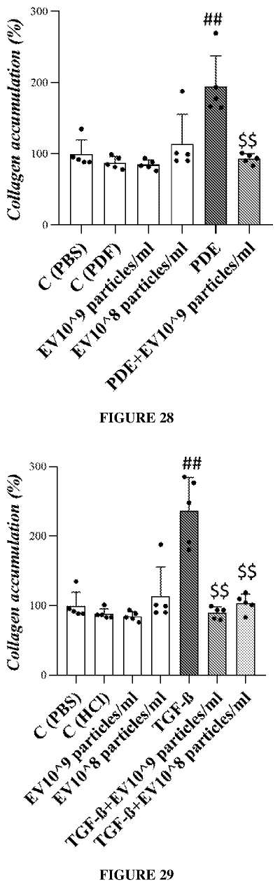

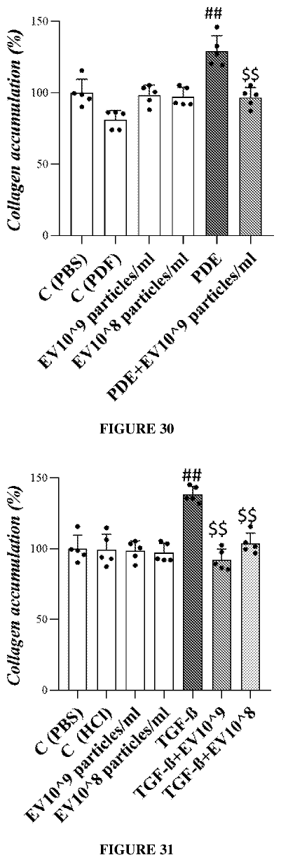

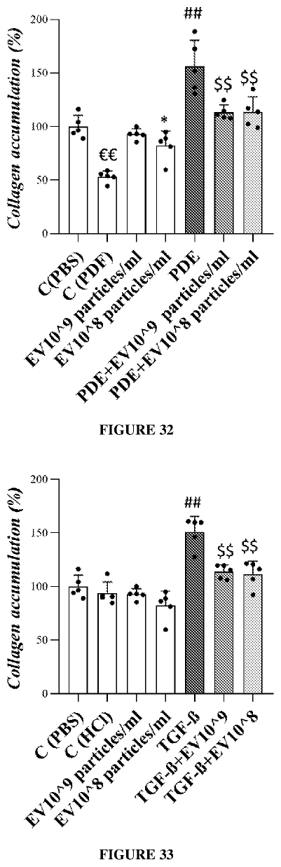

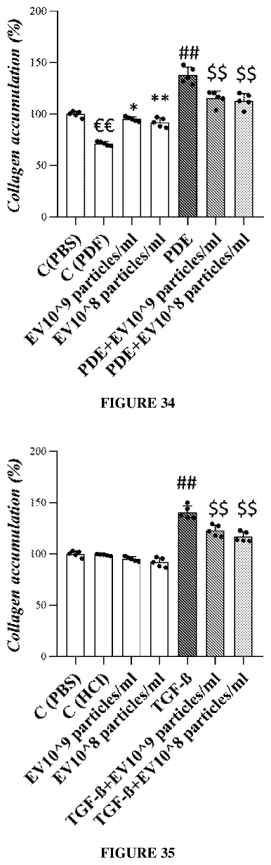

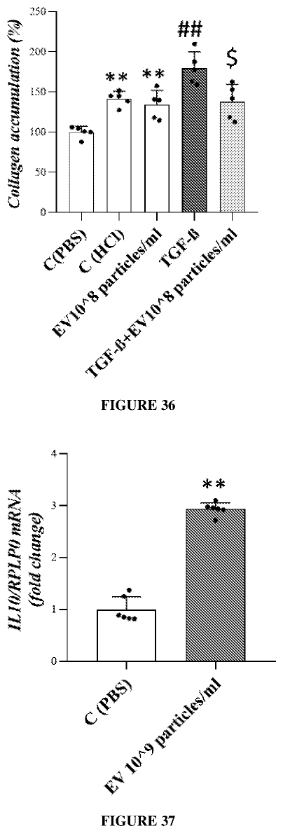

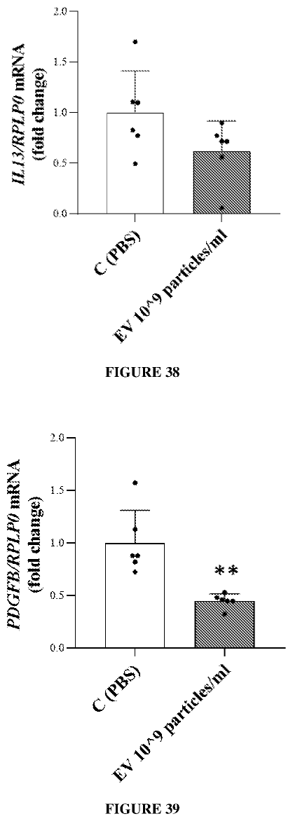

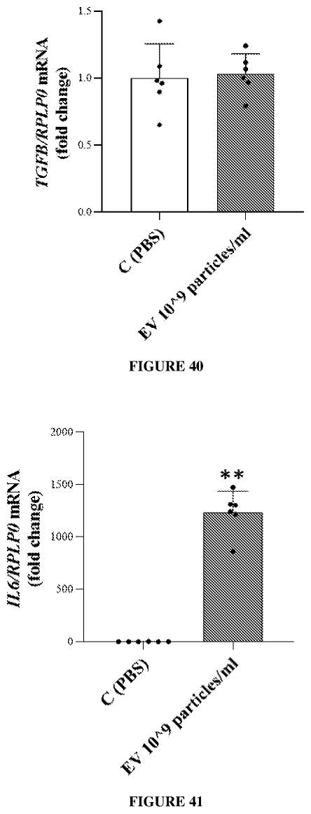

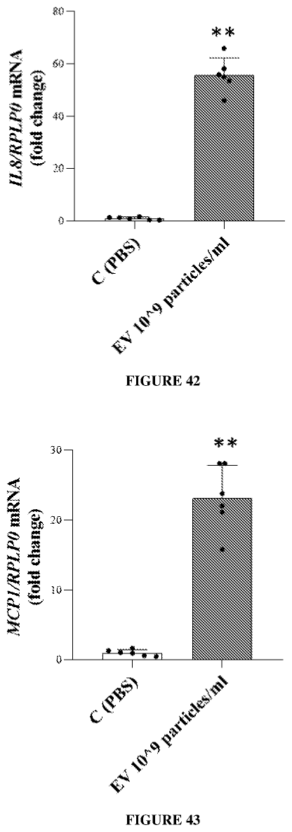

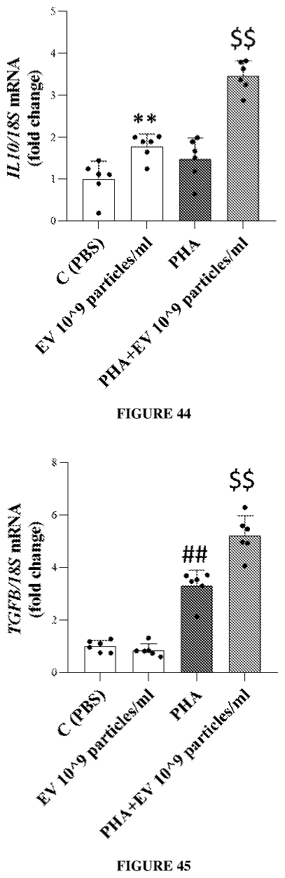

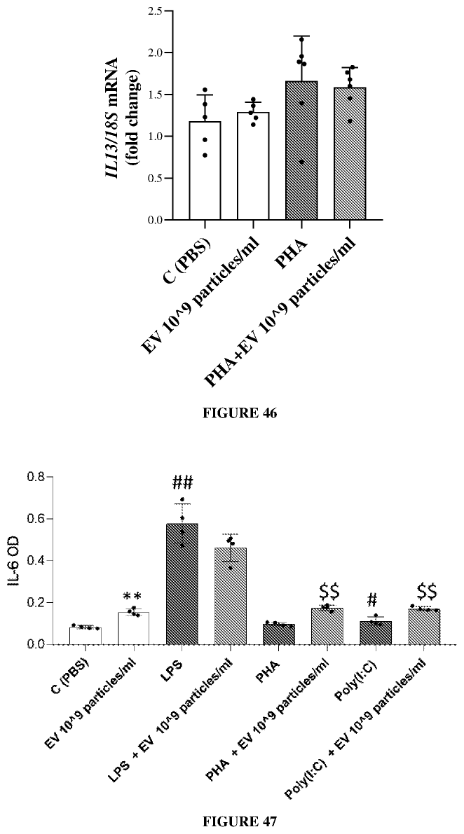

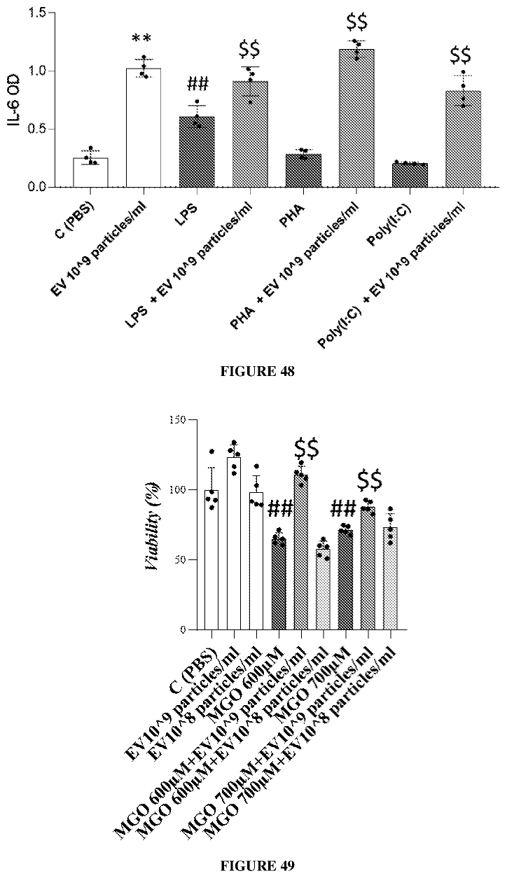

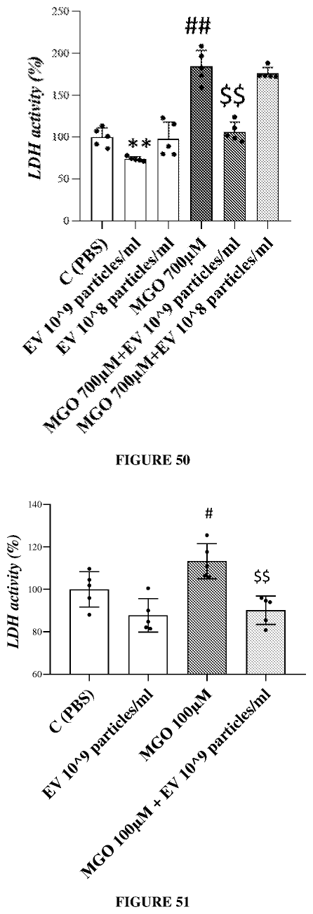

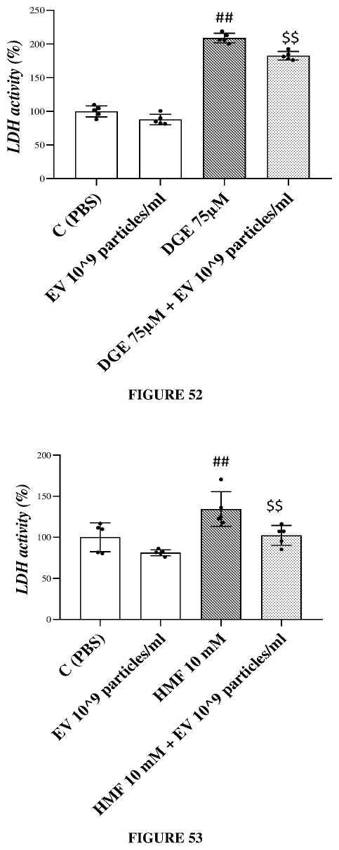

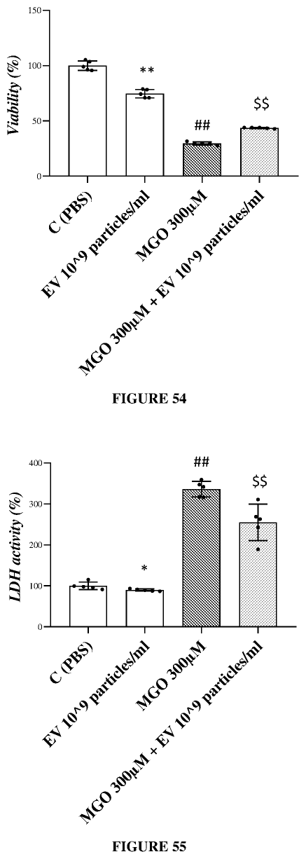

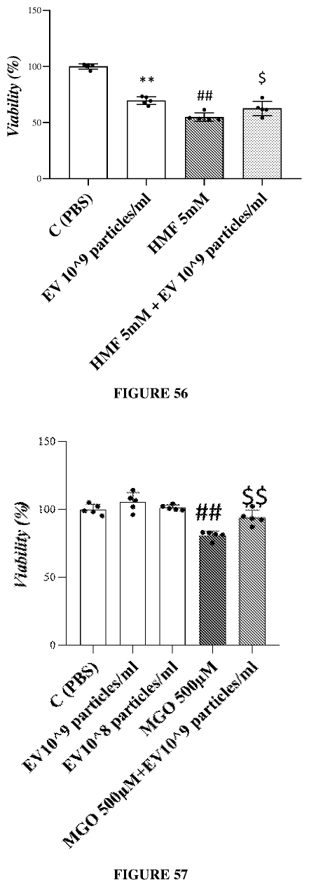

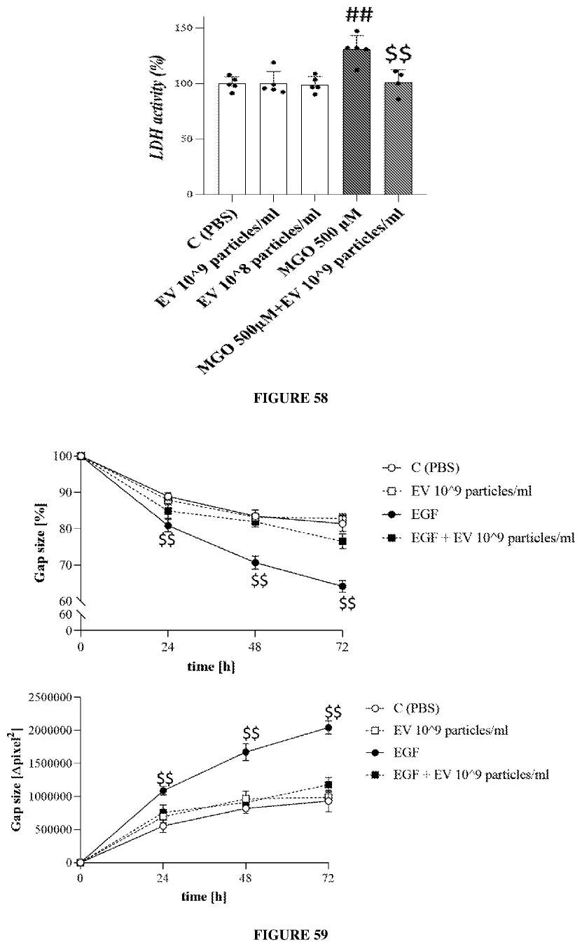

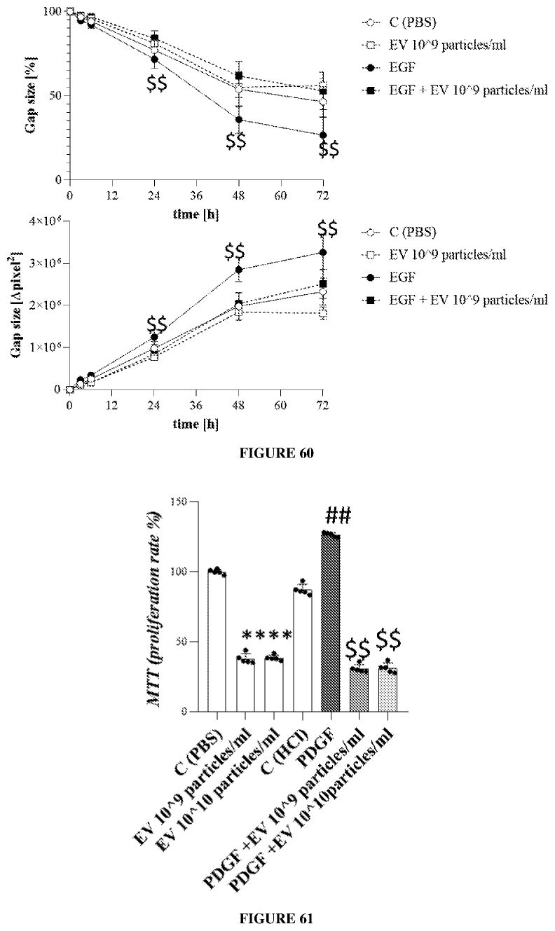

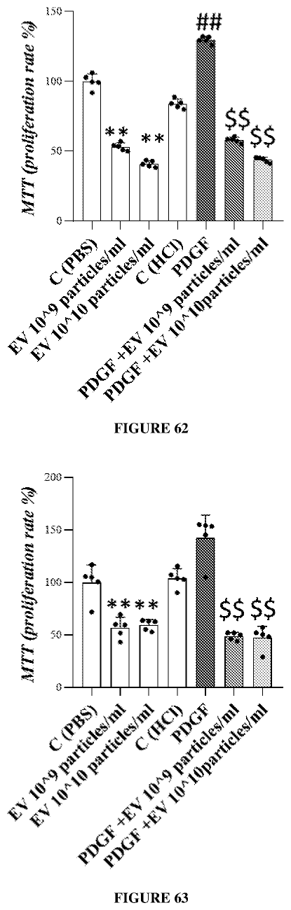

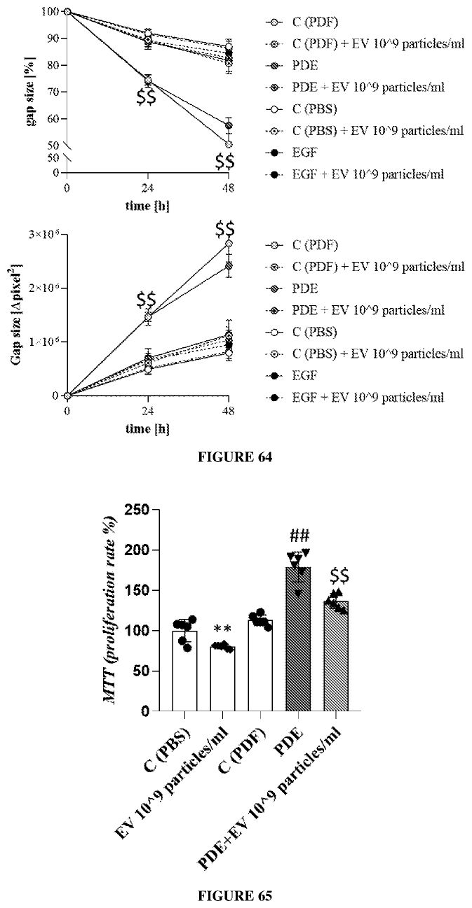

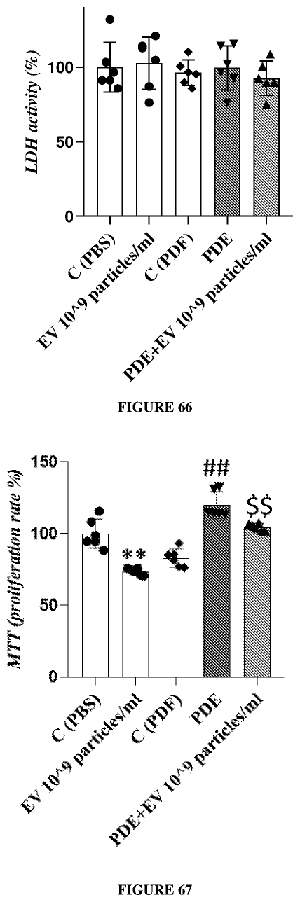

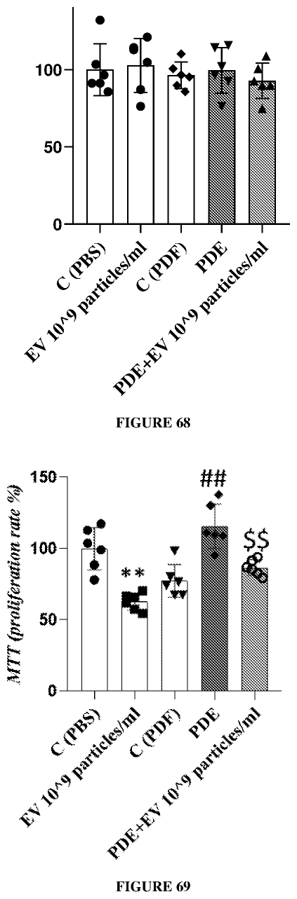

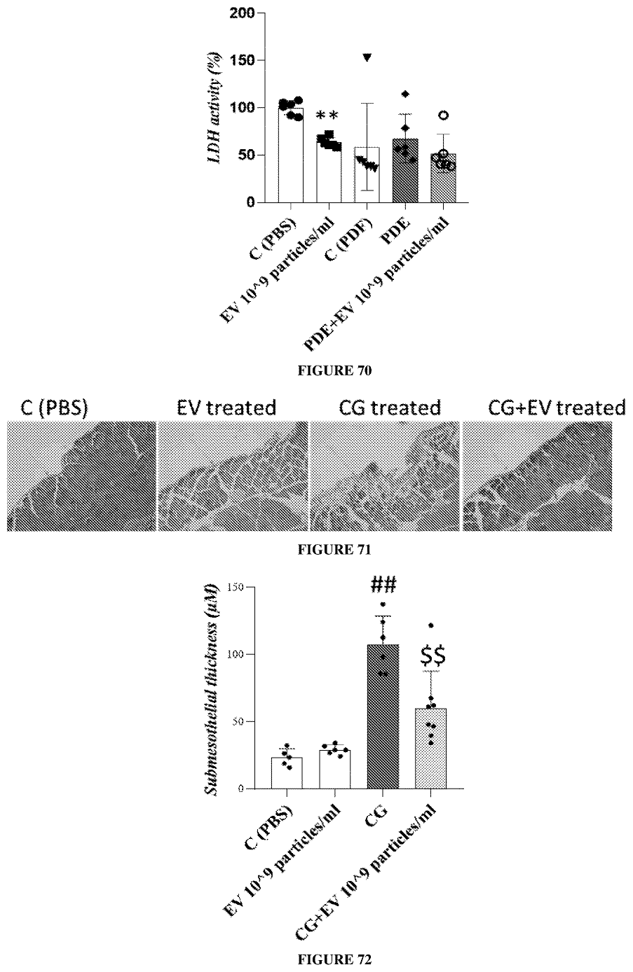

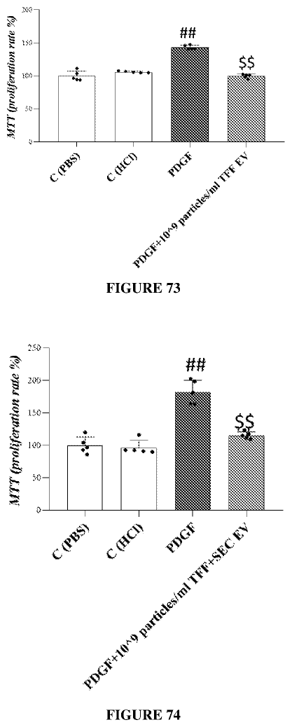

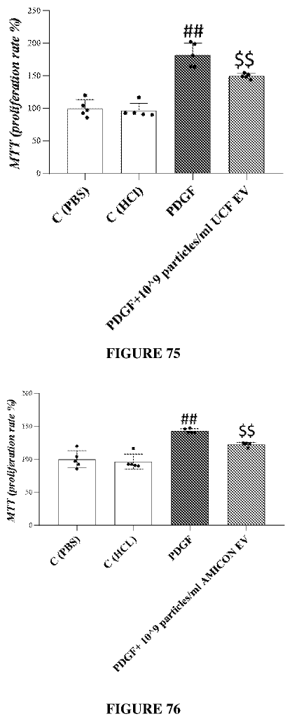

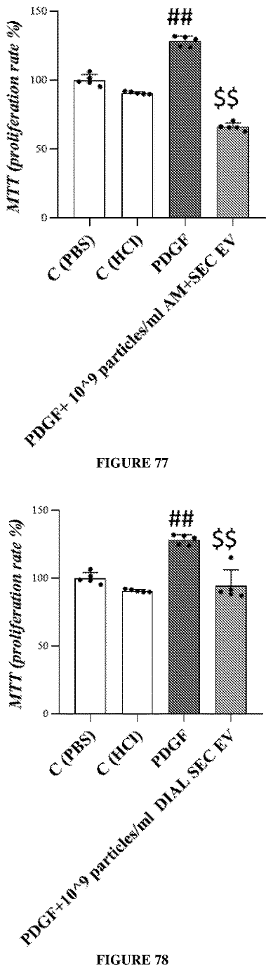

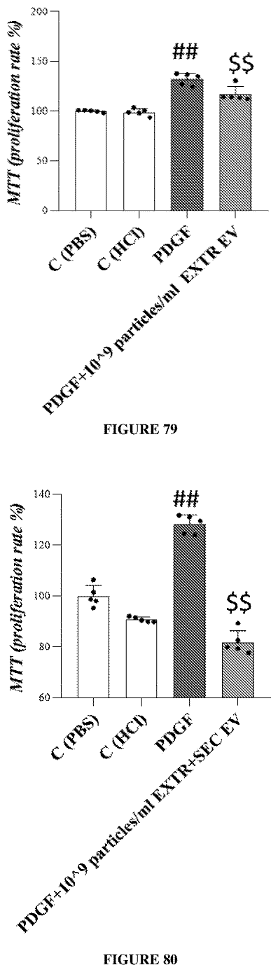

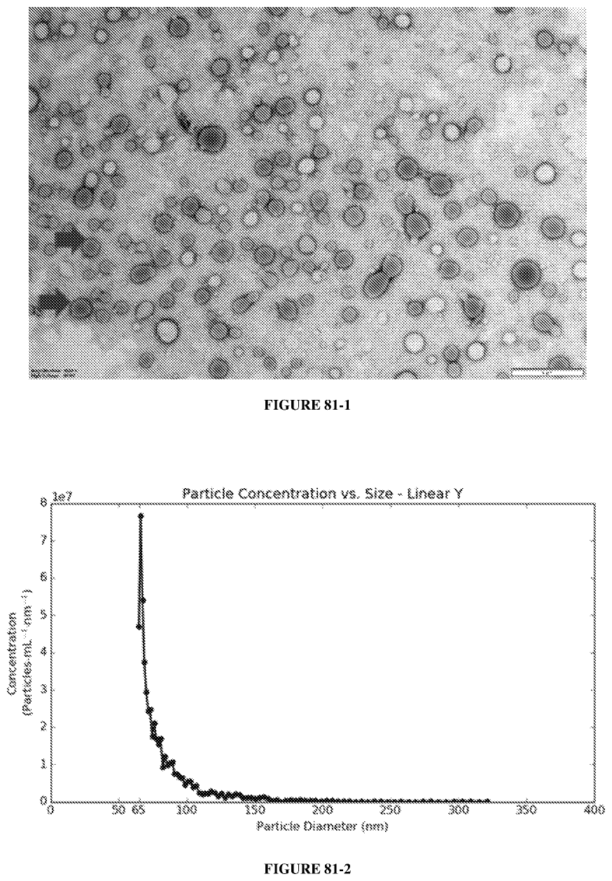

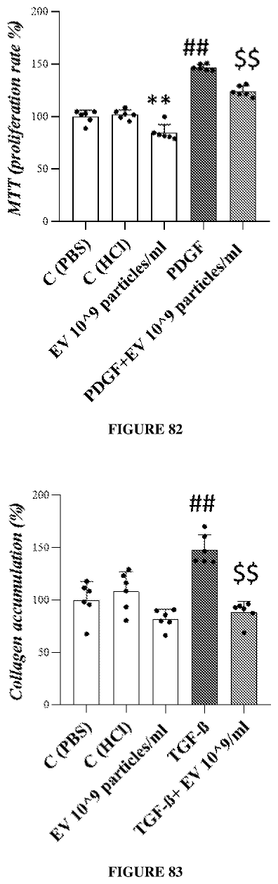

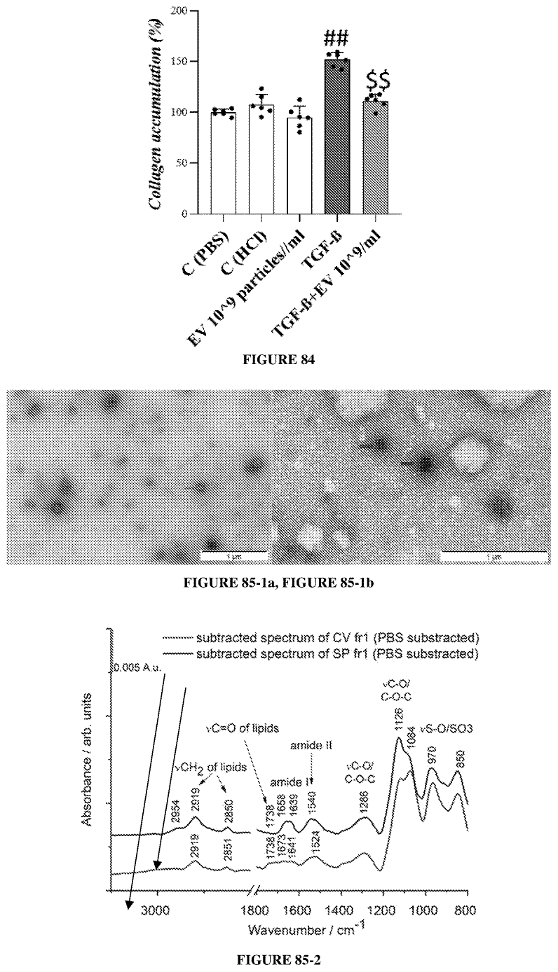

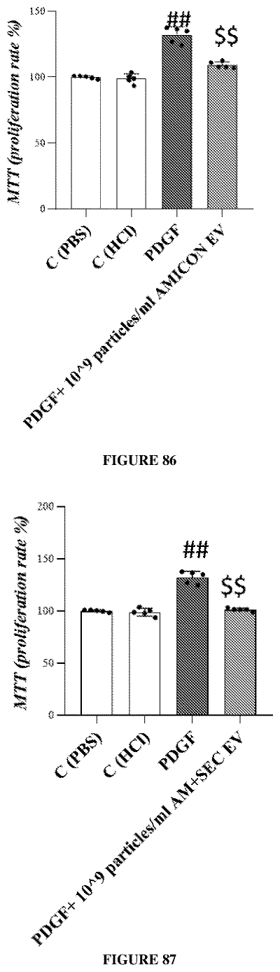

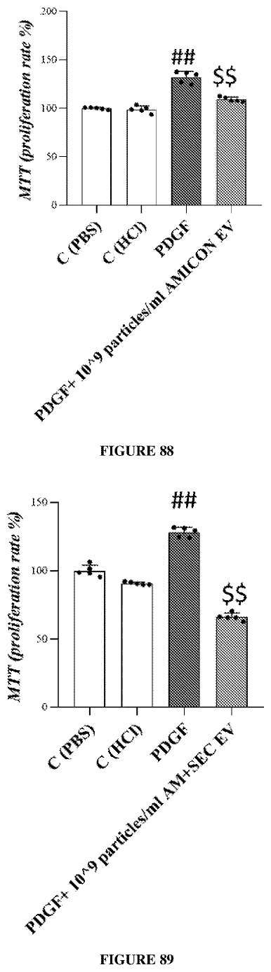

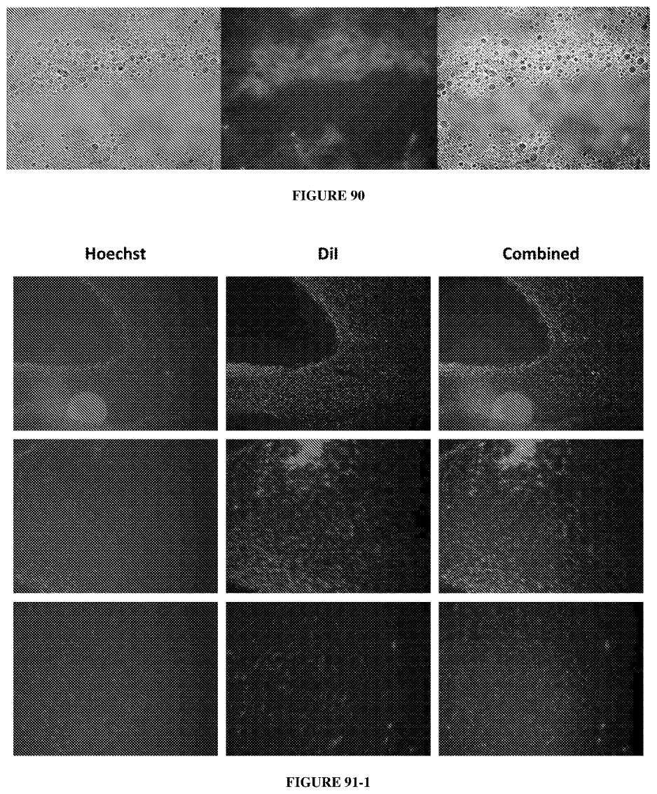

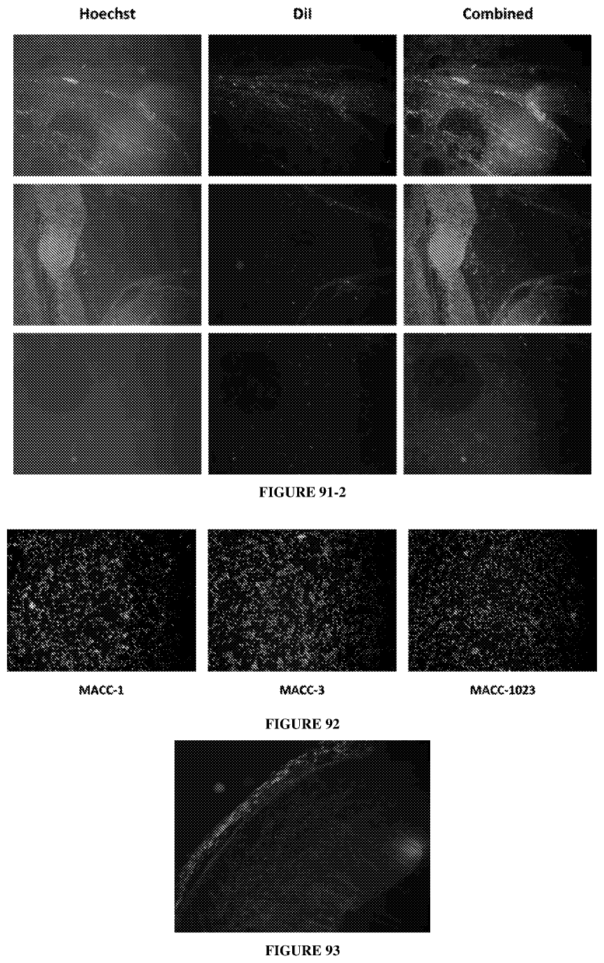

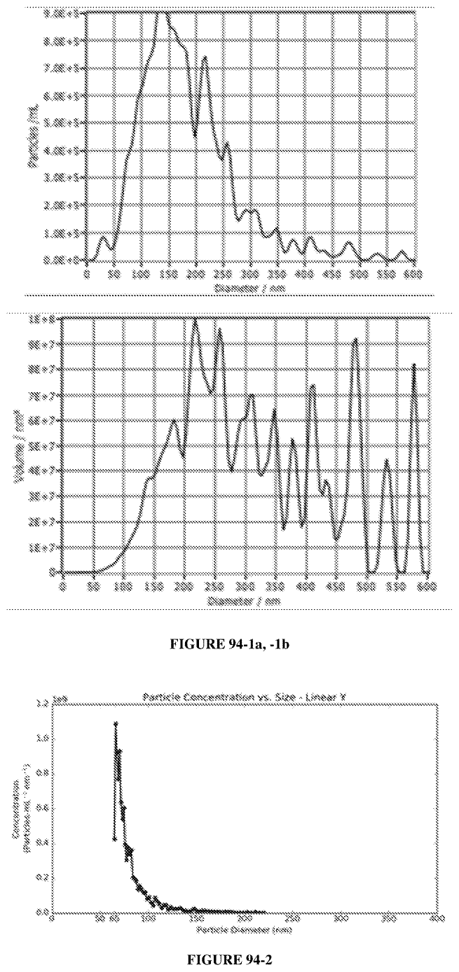

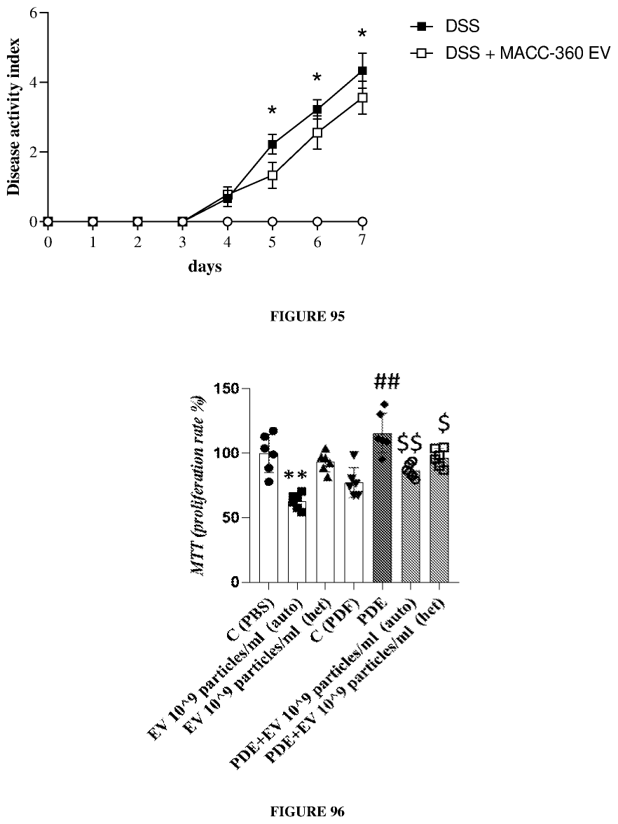

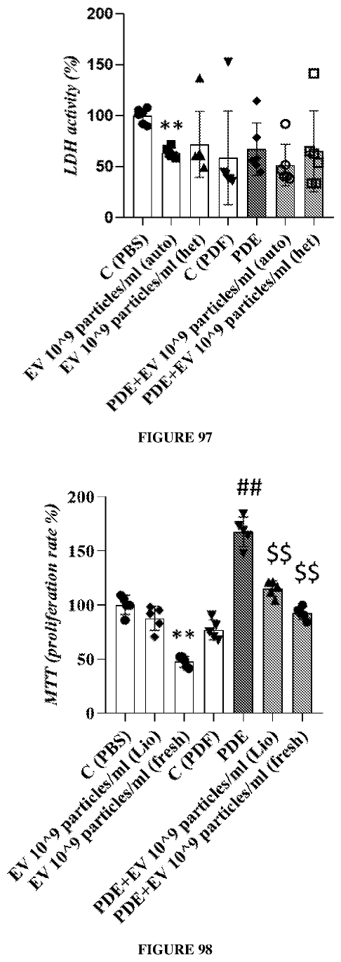

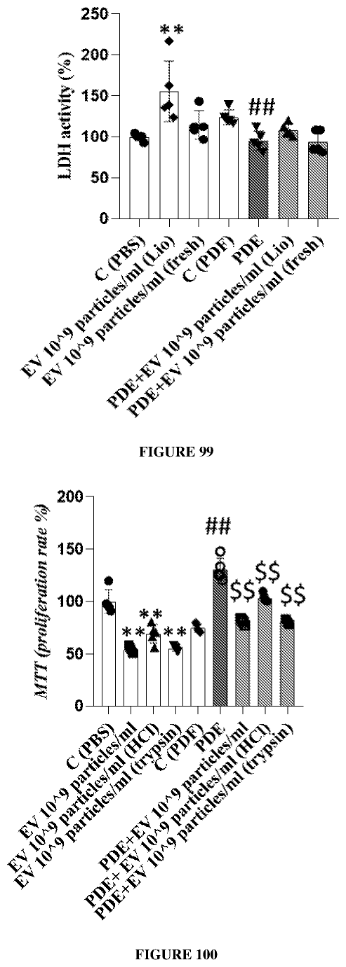

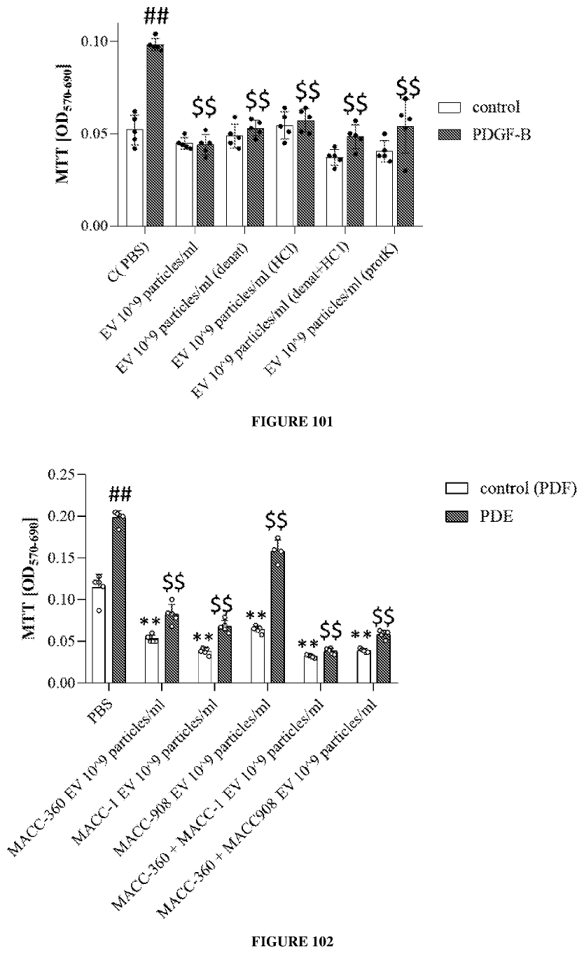

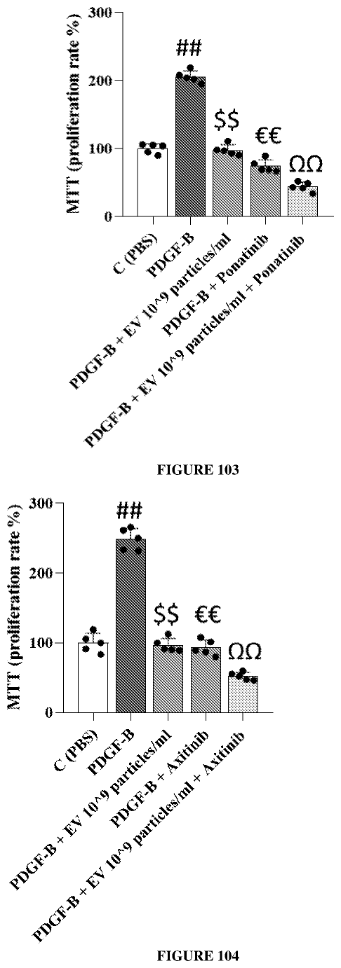

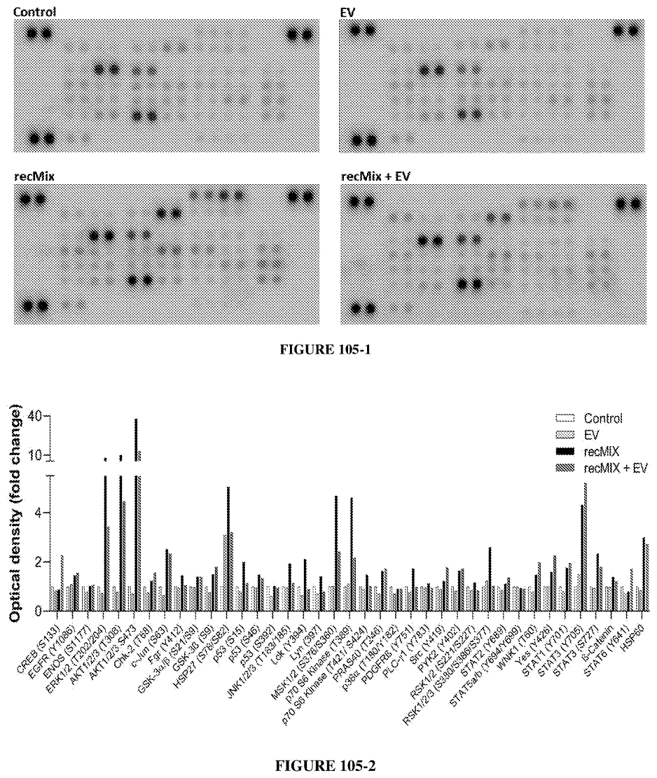

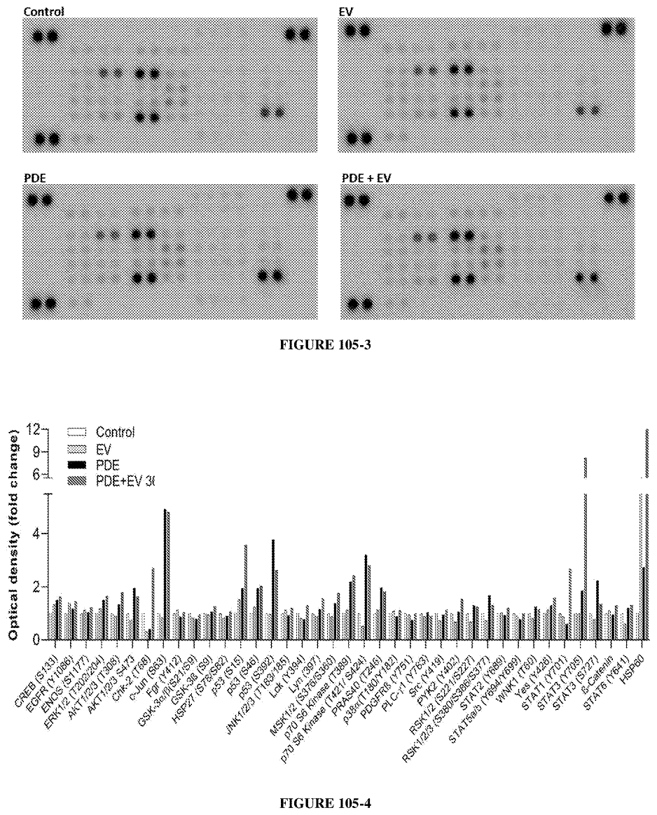

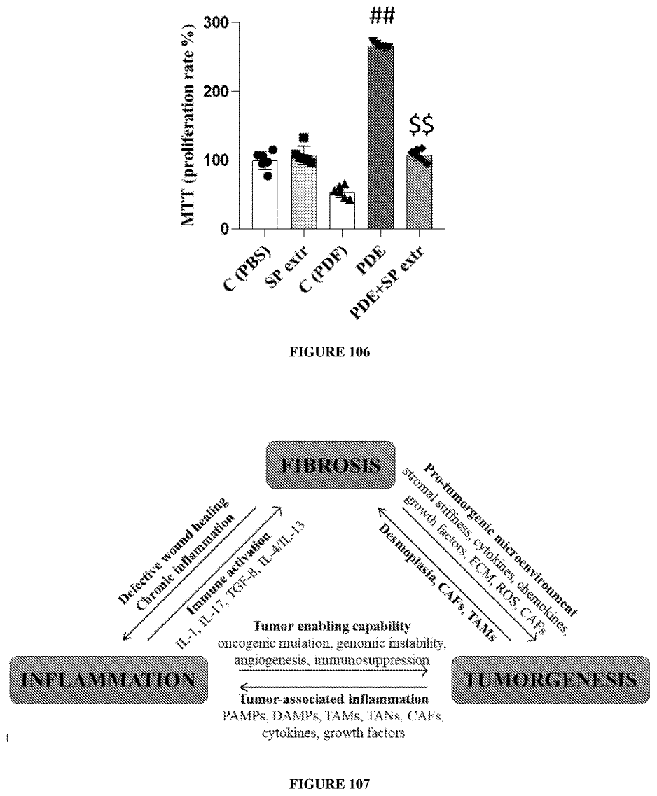

Primary cells were characterized by immunofluorescence staining (FIG. 1-6). The cytoplasm of fibroblasts showed positive staining for specific fibroblast marker α-SMA and negative for mesothelial marker CK18, while mesothelial cells showed CK18 immunopositivity.

EVs were found to penetrate into different cells (FIG. 9-13), making it possible to exert their effects inside and/or on the cells.

Moreover, EVs administered into the tail vain of (C57/BL6 J) mice internalized were targeted and internalized into the liver (FIG. 90). This result suggests that EVs may be particularly useful in the treatment of diseases e.g. fibroproliferative diseases or cancer of the liver.

EVs have been administered either intraperitoneally (FIG. 91-92) or intracutaneously, to the skin (FIG. 93). The effect of EVs on fibroblast migration

EVs were found to inhibit migration of primary colon and skin fibroblasts. The effect of EVs on cell migration was shown on different types of cells in a TAS migration assays. Migration of the various primary fibroblasts was induced by EGF (FIGS. 59 and 46). These experiments show that addition of EVs are capable of inhibiting cell migration in particular, EGF induced cell migration (FIG. 59, 60).

In a further set of experiments migration was initiated with PDE on primary colon and skin fibroblasts and measured by TAS assay. These experiments are shown on Tables 1-1 to 1-4. It can be seen that EVs from several algae migration was significantly reduced and in none of the cases was it increased.

### The Effect of EVs on Fibroblast Proliferation

EVs were found to inhibit cell proliferation. Proliferation of the various primary fibroblasts was induced by PDE (FIGS. 14, 16, 18, 20, 22, 24) or PDGF-BB (FIGS. 15, 17, 19, 21, 23, 25), and EVs of Chlorella MACC-360 Chlorella (Chlorella pyrenoidosa) (FIGS. 88-89), Spirulina (Spirulina platensis) (FIGS. 86-87) and Parachlorella (Parachlorella kessleri) (FIGS. 58-60) were able to inhibit this effect as measured in an MTT assay.

In a further set of experiments fibroblast proliferation was initiated with PDE on primary colon and skin fibroblasts and proliferation was measured by MTT assays. These experiments are shown on Tables 1-1 to 1-5. It can be seen that EVs from each algae significantly reduced fibroblast proliferation and thus provided an antifibrotic effect.

### Effect of EVs on ECM Production of Fibroblasts

EVs of different origin were found to exert a strong effect on the ECM production of various primary fibroblasts tested. Collagen production was induced by PDE (FIGS. 26, 28, 30, 32 and 34) or TGF-β (FIG. 27, 29, 31, 33, 35, 36, 63, 64). Different concentrations of EVs inhibited the PDE or TGF-β induced increase in collagen production. In a further set of experiments collagen accumulation was stimulated with PDE on primary colon and skin fibroblasts. These experiments are shown on Tables 1-1 to 1-5. It can be seen that EVs from each algae significantly reduced collagen production and thus provided a clear and general antifibrotic effect.

### The Effect of EVs on the Production of Profibrotic Factors of Immune Cells.

EVs inhibited PDGF-B gene expression (FIG. 39) of peripheral blood mononuclear cells (PBMC), indicating their antifibrotic potential.

### In Vivo Antifibrotic Effect of the EVs.

Moreover, the antifibrotic effect of EVs was also examined in vivo, in the chlorhexidine gluconate (CG) induced peritoneal fibrosis model of C57Bl/6 mice. EVs of MACC-360 origin significantly inhibited the increase of the submesothelial thickness induced by CG. Submesothelial thickness is in positive correlation with the severity of peritoneal fibrosis (FIG. 71)

### In Vivo Effect of the EVs in a DSS Induced Colitis Model.

The anti-inflammatory effect of EVs was also examined in vivo, in a DSS induced colitis model of C57Bl/6 mice. Changes in the disease activity index (DAI) were monitored during the whole experiment. At the end of treatment (after 7 days) mice were sacrificed and disease activity index has been measured and recorded. EV-treated mice have shown a markedly improved DAI over control (FIG. X 95)

The effect of EVs on proliferation of cancer cells.

EVs were found to inhibit cancer cell proliferation. Proliferation of the various cancerous cells (A549, HT29, CACO-2, Hs578-T, LCLC-103H, HepG2) was induced by PDGF-BB (FIGS. 47, 48, 49), and EVs of Chlorella MACC-360 were able to inhibit this effect as measured in MTT assays (FIGS. 61-63, 65, 67, 69) and did not induced cell death (FIGS. 66, 68, 70).

In a further set of experiments proliferation was initiated with PDE on HepG2 and Caco-2 cells. These experiments are shown on Tables 1-1 to 1-4. It can be seen that EVs from each algae significantly reduced proliferation of these cells and thus provided a clear and general anticancer effect.

### The Effect of EVs on Cancer Cell Migration.

EVs of Chlorella sp. MACC-360 origin were found to inhibit the migration of Caco-2 colon carcinoma cell cells in TAS migration assay (FIG. 50.). EVs inhibited the PDF and also the PDE induced migration of the Caco-2 cells.

### Anti-Inflammatory Effect of EVs

The anti-inflammatory effect of EVs of MACC-360 origin was demonstrated by the massive induction of IL-6 (FIG. 41), IL-10 (FIG. 37), gene expression of PBMC upon treatment with EVs. Furthermore IL-10 and TGFB gene expression of PBMC was even more evaluated after phytohemagglutinin and EV cotreatment (FIGS. 44-45). Additionally IL-6 protein level was even more emphasised after LPS, PHA, poly (I:C) and EV cotreated primary skin fibroblast (FIG. 48) and PHA, poly (I:C) and EV cotreated adenocarcinoma human alveolar basal epithelial cells (FIG. 47)

### In Vivo Anti-Inflammatory Effect of the EVs in a DSS Induced Colitis Model.

The anti-inflammatory effect of EVs of MACC-360 origin was also examined in vivo, in a DSS induced colitis model of C57Bl/6 mice. Changes in the disease activity index (DAI) were monitored during the whole experiment. At the end of treatment (after 7 days) mice were sacrificed and disease activity index has been measured and recorded. EV-treated mice have shown a markedly improved DAI over control (FIG. 95)

### Cytoprotective Effect of EVs

The cytoprotective effect of EVs was demonstrated in methylglyoxal (MGO) induced cytotoxicity experiments in primary mesothelial cells, primary colon and skin fibroblasts, and human umbilical vein endothelial cell line as well as 3,4-Dideoxyglucosone-3-ene (DGE) and 5-Hydroxymethyl-2-furaldehyde (HMF) induced cytotoxicity experiments in case of primary colon and skin fibroblasts. Viability assay (MTT) (FIGS. 49, 54, 56, 57) and LDH release (LDH assay, FIG. 50-53, 55, 58) both showed EVs to be cytoprotective.

### Use of Alga EVs in Combination

Furthermore, the EVs may be applied in combination, i.e. alga EVs isolated from multiple alga sources and applied together, e.g. in a mix or in a composition may be preferred.

For example, PDE induced proliferation of fibroblasts was more effectively inhibited in MTT assay by a combination of MACC-360 and further alga EVs than single type of EVs alone (see FIG. 102).

In an other embodiment alga EVs are combined with other active agents. It has been surprisingly found that both Ponatinib and Axitinib used together with EVs have reduced PDGF-B induced proliferation of primary human colon fibroblasts that the agents alone (FIGS. 103 and 104).

The Effect of EVs are Maintained after Harsh Environmental Effects

The inventors have also found that the EVs are surprisingly resistant to various environmental effects and method of preparation.

For example denaturated, hydrochloric acid (HCl) treated, denaturated and HCl treated and proteinase K treated samples still had a significant effect on platelet derived growth factor B (PDGF-B) induced proliferation of primary human colon fibroblast as measured in MTT assay (FIG. 101)

Similarly, hydrochloric acid treated and trypsin-EDTA treated samples still had a significant effect on platelet derived growth factor B (PDGF-B) induced proliferation of primary human colon fibroblast as measured in MTT assay (FIG. 100).

The method of cultivation (autotroph and heterotroph) may influence the effect of alga EVs, however, it is plausible that het cultivation may also be used (FIG. 97). Cytotoxicity measurement have not refuted this finding.

### The Method of Isolation has No Effect on the Biological Effects of EVs

EVs were isolated from the supernatant of the algae culture and that of rehydrated algae, and also from the algae itself. FIGS. 73-80 show that the method of isolation had no effect on the biological effects of EVs. Moreover, the antiproliferative effect of EV remained largely unchanged upon lyophilization as shown by on peritoneal dialysis effluent (PDE) induced cytotoxicity of of primary human colon fibroblasts (FIG. 99).

### Effect of EVs on a Kinase Panel

The effect of EV of Chlorella sp. MACC-360 origin on recombinant protein mix (recMix) and peritoneal dialysis effluent (PDE) induced protein kinase phosphorylation in primary human colon fibroblasts was also studied. Representative images of the array membranes are shown on FIG. 105. While different set of kinases have been effected it is clearly shown, as discussed above, that EVs are effective on various kinase pathways activated by growth factors as demonstrated herein in other experiments.

Thus, in the present invention EVs may affect
PDGF receptor alpha and/or PDGF receptor beta signaling pathways, preferably PDGF-induced signaling, preferably PDGF-BB induced signalling pathway,
TGFβ, preferably TGFbeta-1 induced signalling pathways,
EGF induced signalling pathway.

PDE comprises a number of pro-fibrotic growth factors.

| | |
|---|---|
| VEGF | Vascular endothelial growth factor-mediated peritoneal neoangiogenesis in peritoneal |
| | dialysis |
| | Yingfeng Shi 1, Yan Hu 1, Binbin Cui 1, Shougang Zhuang 1 2, Na Liu 1 |
| CTGF | Connective tissue growth factor (CTGF/CCN2) is increased in peritoneal dialysis patients |
| | with high peritoneal solute transport rate |
| | Makoto Mizutani 1, Yasuhiko Ito, Masashi Mizuno, Hayato Nishimura, Yasuhiro |
| | Suzuki. Ryohei Hattori, Yoshihisa Matsukawa, Masaki Imai, Noelynn Oliver, Roel |
| | Goldschmeding, Jan Aten, Raymond T Krediet, Yukio Yuzawa, Seiichi Matsuo |
| FGF21 | Fibroblast Growth Factor 21 (FGF-21) in Peritoneal Dialysis Patients: Natural History and |
| | Metabolic |
| | Implications |
| | Elena González 1, Juan J Díez 2, M Auxiliadora Bajo 1, Gloria del Peso 1, Cristina Grande |
| | 2, Olaia |
| | Rodríguez 3, Mariana Díaz-Almirón 4, Pedro Iglesias 2, Rafael Selgas 1 |
| FGF23 | Fibroblast Growth Factor 23 in Patients Undergoing Peritoneal Dialysis |
| | Tamara Isakova,* Huiliang Xie,* Allison Barchi-Chung,* Gabriela Vargas,* Nicole |
| | Sowden,* Jessica |
| | Houston,* Patricia Wahl.* Andrew Lundquist,† Michael Epstein,† Kelsey Smith,* Gabriel |
| | Contreras,* |
| | Luis Ortega,* Oliver Lenz,* Patricia Briones,* Phyllis Egbert,‡ T. Alp Ikizler,‡ Harald |
| | Jueppner,§ and |
| | Myles Wolfcorresponding author* |
| TGF-β1 | Growth factors VEGF and TGF-beta1 |
| | in peritoneal dialysis |
| | M M Zweers 1, D R de Waart, W Smit, D G Struijk, R T Krediet |
| HB-EGF | HB-EGF is produced in the peritoneal cavity and enhances mesothelial cell adhesion and |
| | migration |
| | Author links open overlay panelRandall J. Faull, Jodie M. Stanley, Scott Fraser. David |
| | A. Power, David I, Leavesley |
| PDGF, EGF | Growth factors in continuous ambulatory peritoneal dialysis effluent. Their relation with |
| | peritoneal transport of small solutes |
| | K N Lai 1, K B Lai, C C Szeto, C W Lam, J C Leung |
| IGF-I, IGF-II, | The insulin-like growth factor system in human peritoneal fluid: its effects on endometrial |
| IGFBP-I, II, III, IV | stromal cells and its potential relevance to endometriosis |
| | L C Giudice, B A Dsupin, S E Gargosky, R G Rosenfeld, J C Irwin |
| TGF-b, PDGF, EGF | Growth Factors in Continuous Ambulatory Peritoneal Dialysis Effluent |
| | Kar Neng Lai, Ka Bik Lai, Cheuk Chun Szeto, Christopher W. K. Lam, Joseph C. K |
| Galectin 3 | GALECTIN-3. IS IT A NEW PLAYER IN PERITONEAL INFLAMMATION AMONG |
| | PATIENTS UNDERGOING PERITONEAL DIALYSIS? |
| | Yael Einbinder, Keren Cohen-Hagai, Sydney Benchetrit, Tali Zitman-Gal |
| MMP2, MMP3, | Matrix metalloproteinase levels in the drained dialysate reflect the peritoneal solute |
| TIMP1 | transport rate: a multicentre study in Japan |
| | Ichiro Hirahara, Makoto Inoue, Tetsuo Umino, Osamu Saito, Shigeaki Muto, Eiji Kusano |

### EVs are Suitable for Use in the Prevention or Treatment of Fibrosis

Our result directly show that EVs are suitable for use in the prevention or treatment of a condition, disorder or disease associated with or characterized by fibrosis, such as a fibroproliferative condition, disorder or disease or a proliferation/migration condition, disorder or disease, including non-physiological ECM production/deposition e.g. in the prevention or treatment of a condition, disorder or disease listed below.

Examples of renal diseases associated with or characterized by fibrosis: diabetic nephropathy, hypertensive nephropathy, glomerular diseases including proliferative glomerulonephritis (mesangial proliferative, membranoproliferative, focal proliferative, diffuse proliferative, crescenic), glomerulonepritis associated with lupus nephritis, bacterial endocarditis, vasculitis, chronic hepatitis, infections (e.g. hantavirus), non-inflammatory glomerular diseases (minimal change nephritis, focal glomerular sclerosis, membranousus nephropathy, fibrillary glomerular disease), glomerular disease associated with Hodgkin's disease, antibiotic, drug (aspirin, ibuprofen, acetaminophen, tacrolimus, cyclosporine, contrast agents, chemotherapy, or heroin toxicity) HIV infection. Hereditary nephritis (Alport syndrome), vascular diseases including renal artery stenosis, sickle cell disease, hemolytic uremic syndrome, atypic hemolytic uremic syndrome. Tubulointerstitial diseases including pyelonephritis, analgesic nephritis, allergic interstitialnephritis, granulomatous interstitialnephritis, autoimmune interstitial nephritis, non-inflammatory diseases like reflux nephropathy, obstructive uropathies (anatomical abnormalities e.g. posterior urethra valve, or stones, or malignancy or prostatism) myeloma kidney, Diseases in the transplant like chronic rejection, drug toxicity, recurrent disease, transplant glomerulopathy,

Examples of lung diseases associated with or characterized by fibrosis: bronchitis, asthma, idiopathic pulmonary fibrosis, usual interstitial pneumonia, gas or ionizing radiation induced lung fibrosis, nitrofurantoin, tobacco smoke-induced lung fibrosis, emphysema, chronic obstructive pulmonary disease, tuberculosis, rheumatoid arthritis induced lung fibrosis, systemic lupus erythematosus induced lung fibrosis, sarcoidosis, Wegener's granulomatosis, nonspecific interstitial pneumonitis, Hamman-Rich Syndrome, diffuse fibrosing alveolitis, inhalation of environmental and occupational pollutants (fume silica, asbestos, nitrogen, and sulfur gases, fumes, vapors of detergents, cleaners, hydrochloric acid, herbicide, hairspray), Drugs-induced pulmonary fibrosis (bleomycin, amiodarone, busulfan, methotrexate, apomorhpine, nitrofuratoin, phenytoin) and radiotherapy, Torque teno virus, pneumoconiosis, etc.

Examples of pancreatic diseases associated with or characterized by fibrosis: alcoholic chronic pancreatitis, hereditary pancreatitis, autoimmune pancreatitis, obstructive chronic pancreatitis, tropical calcific pancreatitis, fibrocalculous pancreatic diabetes, chronic non-alcoholic pancreatitis, chronic atrophic pancreatitis, Groove pancreatitis,

Examples of intestinal diseases associated with or characterized by fibrosis: ulcerative colitis, Crohn's disease, Collagenous colitis, microscopic colitis, diversion colitis, necrotizing enterocolitis, chemical colitis, ischemic enterocolitis, Helicobacter pylori-induced gastritis, chronic gastritis, Oesophageal subepithelial fibrosis, Barrett's esophagus, gastroesophageal reflux disease, oral submucous fibrosis, oesophageal atresia,

Examples of hepatic diseases associated with or characterized by fibrosis: nonalcoholic steatohepatitis, autoimmune hepatitis, viral hepatitis (hepatitis A, hepatitis B, hepatitis C, hepatitis D), alcoholic hepatitis, toxic and drug-induced hepatitis, non-alcoholic fatty liver disease, liver cirrhosis, fascioliasis, schistosomiasis, liver fluke induced fibrosis, primary sclerosing cholangitis, Budd-Chiari syndrome, biliary atresia, Alagille syndrome, progressive familial intrahepatic cholestasis, serotonergic agonist drugs: weight loss drugs (fenfluramine, chlorphentermine, aminorex), anti-migraine drugs (ergotamine, methysergide), antiparkinsonian drugs (pergolide, cabergoline), recreational drugs (MDA, MDMA, DOI, mCPP),

Examples of eye diseases associated with or characterized by fibrosis: diabetic retinopathy, fibrosis of the cornea, neovascular glaucoma, retinopathy of prematurity, age-related macular degeneration, premacular fibrosis, herpetic keratitis, pingueculae, capsular fibrosis, fibrosis of the posterior lens capsule, fibrovascular scarring of the retina, gliosis in the retina, complication of surgery to treat retinal detachment, viral infection of the cornea, retinal injury due to hypoxia or inflammatory changes, trachoma, congenital fibrosis syndrome, levator muscle fibrosis, congenital fibrosis of the ocular muscles, congenital fibrosis of the extraocular muscles, proliferative retinopathy, macularfibrosis, talc retinopathy, subretinal fibrosis, syndrome, sub-conjunctival fibrosis, Examples of metabolic diseases associated with or characterized by fibrosis: type 2 diabetic complications atherosclerosis, arteriosclerosis, diabetic foot, metabolic syndrome, hyperlipidaemia, haemochromatosis, Wilson-disease, alfa-1-antitrypsin deficiency, galactosaemia, glycogen storage disease I-IV, VI, IX, XI, urate nephropathy, hyperlipoproteinaemia I.-V., familiar hypercholesterineaemia, mucopolysaccharidosis type I-VII., mucolipidosis III-IV, Fabry disease (angiokeratoma corporis diffusum), pseudoxanthoma elasticum, Examples of autoimmune diseases associated with or characterized by fibrosis: Type 1 diabetic complications, rheumatoid arthritis, ankylosing spondylitis (Bechterew's disease), systemic lupus erythematosus, systemic sclerosis, Sjögren's syndrome, CREST-syndrome, polymyositis, dermatomyositis, primary biliary cirrhosis, primary sclerotising cholangitis, vasculitis: giant cell arteritis, Takayasu's arteritis, polyarteritis nodosa, Wegener's granulomatosis, thromboangitis obliternas, sarcoidosis, Goodpasture syndrome, mixed connective tissue disease, Churg-Strauss-syndrome,

Examples of skin diseases associated with or characterized by fibrosisE keloid and scars associated with trauma, operations, piercing, acne, chicken pox, infections, cutting, haematoma, spontaneusly, granuloma, tick-granuloma, solaris atrophia, burn injury, pseudocicatrix stellata (Batman purpura), ulcus associated with anthrax, gonorrhoea, ulcus molle, tularaemia, decubitus, diabetic foot ulcer, diabetes skin, necrobiosis lipoidica diabeticorum, varicosits cruris, thrombophlebitis, infections: fascitis necrotisans, ecthyma simplex, ecthyma gangrenosum, phlegmone-abscessus, furunculus, carbunculus, anthrax, granuloma venereum, tularaemia, tbc (lupus vulgaris, scrofuloderma), lepra, Lyme-borreliosis, Tibola (Tick-Bone-Lymphadenopathy), syphilis, actinomycosis, every mycotic infection secundary infection scar tissue, HSV, VZV, erythema multiforme, dermatitis herpetiformis, scars associated with prurigo (infection, allergy, irritation, paraneopl.gravidarum, diabetes) acne: ecthyma simplex, acne inversa, acne vulg, rosacea, rinophima, dermatitis seborhoica, Cushing-syndrome), Operations, side effects of surgery (sec. infection, sponge, splintering),

Examples of diseases of the urogenital tract associated with or characterized by fibrosis: menstrual disorders: endometriosis, PCOS, adrenal diseases (CAH, Cushing, virilizing sy, acne, seborrhea), Asherman's syndrome (-iatrogen), endometritis, IUD), infections: perinephritis, paranephritis, pyelonephritis, pyelitis and pyelonephritis. chronica., pyelonephros, chonic. uretritis (gonorrhoea, E. coli, Proteus, HSV), retroperitoneal fibroma., cystitis chronica., cystitis after radiotherapy, ulcus simplex (Hunner), Trichomonases, tuberculosis (renis, vesicae urinariae, epididymitis, prostata), actinomycosisulcus, pelveopeitonitis, vulvovaginitis cand., herpes genitalis, genitalis HPV, chronic cervicitis, endometritis, salpingitis, abscessus, tuboovarii, syphilis, gonorrhoea, chlamidya, trichomonas, HPV, ulcus molle, HIV, tuberculosis,

Examples of fibroproliferative diseases associated with pathological pregnancy associated with or characterized by fibrosis: pruritus gravidarum, bullosus pemphigoid, impetigo herpetiformis, caesarian section (or other operation) rupture corporis uteri, ulcer puerperalis, endometritis, myometritis puerperalis, adnexitis puerperalis, pelveoperitonitis puerperalis, parametritis puerperalis, thrombophlebitis, mastitis puerperalis. in men: penis, prostata, orchis: cavernitisi, induratio penis plastica, prostatitis, abscessus, orchitis chronic. epididymitis. Obstructive uropathies associated with anatomical abnormalities posterior urethra valve, subvesical obstruction, vesicouretheral reflux nenhrolithiasis, inflammation, arthritis urica, hyperparathyreosis, hypercalcaemia, oxalosis, cystinuria, xantinuria.

Examples of cardiovascular diseases associated with or characterized by fibrosis:

Dilated and hypertrophic cardiomyopathies, myocardial infarction, valvular diseases, arrhythmia, cardiac hypertrophy, hypertension induced cardiac fibrosis, Marfan syndrome, left ventricular fibrosis, myocardial necrosis and apoptosis induced cardiac fibrosis, vascular fibrosis, arteriosclerosis, atherosclerosis, venosclerosis Examples of skeletal muscle system diseases associated with or characterized by fibrosis: myelofibrosis, muscle fibrosis.

Examples of central nervous system diseases associated with or characterized by fibrosis: stroke and tissue injury induced glial fibrosis, Parkinson's diseases, amyotrophic lateral sclerosis, multiple sclerosis, Alzheimer's disease.

The role of PDGFR inhibitors in the treatment of fibrotic diseases, e.g. lung fibrosis, myelofibrosis, systemic sclerosis, nephrogenic systemic fibrosis is clinically proven. Our results indicate that EVs may be used similarly to PDGFR inhibitors in the treatment of fibrotic diseases. For example pegpleranib, an anti-PDGF aptamer is implicated in the treatment of sub-retinal fibrosis and subfoveal fibrosis, E10030, another anti-PDGF aptamer in the treatment of macula degeneration. Our results indicate that EVs may be used similarly to anti-PDGF aptamer drugs in the treatment of fibrotic diseases.

The role of TGF-b neutralizing antibodies in the treatment of fibrotic diseases, e.g. that of fresolimumab in the treatment of systemic sclerosis is clinically tested. Our results indicate that EVs may be used similarly to TGF-b neutralizing antibodies in the treatment of fibrotic diseases.

The role of EGFR neutralizing antibodies in the treatment of fibrotic diseases, e.g. that of nimotuzumab in the treatment of COVID-19 induced lung fibrosis is clinically tested. Our results indicate that EVs may be used similarly to EGFR neutralizing antibodies in the treatment of fibrotic diseases.

### PDGF Neutralizing? . . . .

Methylglyoxal (MGO) is a potent reactive carbonyl species and precursor for the formation of advanced glycation end products (AGEs) by reaction with free amino groups of lysine and arginine. Thus, experiments with MGO provide a model for effect of AGEs and possible reversal thereof. [Leone, Alessia et al. “The Dual-Role of Methylglyoxal in Tumour Progression—Novel Therapeutic Approaches” Frontiers in Oncology 2021 (11) DOI=10.3389/fonc.2021.645686]. AGEs induce excess accumulation of ECM and expression of inflammatory and profibrotic cytokines. In addition, studies on receptors for advanced glycation end products (RAGE) have shown that the ligand-RAGE interaction activates several intracellular signalling cascades associated with several fibrotic diseases. AGEs induce excessive deposition of ECM and enhance expression of profibrotic cytokines, including TGF-β (Kyung S Y, Byun K H, Yoon J Y, Kim Y J, Lee S P, Park J W, Lee B H, Park J S, Jang A S, Park C S, Jeong S H. Advanced glycation end-products and receptor for advanced glycation end-products expression in patients with idiopathic pulmonary fibrosis and NSIP. Int J Clin Exp Pathol. 2013 Dec. 15; 7 (1): 221-8. PMID: 24427342; PMCID: PMC3885476.). EVs were found to exert a protective effect against MGO induced cytotoxicity in our experiments, and therefore are good candidates for use in the treatment of AGE induced/related diseases including fibrotic diseases.

Inflammation is an important trigger for fibrosis, and many other diseases including infection/inflammatory/autoimmune diseases (Mack: Inflammation and fibrosis. Matrix Biol. (2018) 68-69, 106-121). The anti-inflammatory effect demonstrated in our experiments also support that EVs are suitable for use in the prevention or treatment of fibrosis and inflammation (in vivo DSS modell)? These experiments suggest that the effect of AGEs may be reversed by algal EVs and their use may be beneficial in diseases developed by AGEs via AGE receptors, such as fibrosis, cancer and inflammation, as well as possibly in autoimmune diseases, diabetes and atherosclerosis, chronic degenerative diseases, such as Alzheimer's disease, Parkinson's disease, and alcoholic brain. [Byunab, Kyunghee et al., “Advanced glycation end-products produced systemically and by macrophages: A common contributor to inflammation and degenerative diseases” Pharmacology & Therapeutics Volume 177, September 2017, Pages 44-55].

### Antitumour Effect of EVs

Our findings that EVs inhibit cell proliferation and cell migration indicate that EVs can be used in anticancer therapy. EVs may be used in a method for the prevention, treatment/inhibition of tumour growth or metastases, and by decreasing the activation of CAF and therefore decrease the pressure within the tumour may also facilitate the antitumour drugs to reach the tumour cells, thus potentiate the effect of anticancer treatments.

EVs may be used in a method for the prevention or treatment of cancer, preferably wherein the cancer is selected from: Acoustic neuroma, Acute lymphoblastic leukaemia, Acute myeloid leukaemia, cancer, melanoma, prostate cancer, Anal cancer, Anaplastic large cell lymphoma, Anaplastic thyroid cancer, Angioimmunoblastic, T-cell lymphoma, Anorectal melanoma, Astrocytoma, Bile duct cancer (cholangiocarcinoma), Bladder cancer, Blood cancer, Bone cancer, Bowel cancer, Brain cancer, Breast cancer, Breast cancer in men, Burkitt Lymphoma, Cancer of unknown primary Cervical cancer, Chondrosarcoma Chordoma, Chronic, lymphocytic leukaemia, Chronic myeloid leukaemia, Colon cancer, Craniopharyngioma, Cutaneous T-cell lymphoma Diffuse large B-cell lymphoma Ductal carcinoma in situ, Early (localised) prostate cancer Ependymoma Essential thrombocythaemia Ewing sarcoma, Eye cancer (ocular melanoma), Fallopian tube cancer, Follicular lymphoma, Gallbladder cancer, Gastrointestinal stromal tumour, Germ cell ovarian cancer, Glioma, Haemangioblastoma, Head and neck cancer, Hodgkin lymphoma, Inflammatory breast cancer, Laryngeal (larynx) cancer, Leiomyosarcoma, Leukaemia, Liver cancer, Locally prostate cancer, Lung cancer, Lymph node cancer, secondary Lymphoblastic lymphoma, Lymphoma MALT lymphoma, Mantle cell lymphoma, Medullary thyroid cancer, Medulloblastoma, Melanoma, Meningioma, Mesothelioma, Mouth cancer, Myelodysplasia, Myelofibrosis, Myeloma, Nasal and sinus cancer, Nasopharyngeal cancer, Neuroendocrine tumours, Nodal marginal zone b-cell lymphoma, Non-Hodgkin lymphoma, Non-small cell lung cancer, Oesophageal cancer, Oligodendroglioma, Oropharyngeal cancer, Osteosarcoma, Ovarian cancer, Paget's disease of the breast, Pancreatic cancer, Papillary and follicular thyroid cancer, Parathyroid cancer, Penile cancer, Peripheral T-cell lymphoma, Phyllodes tumours, Pineal region tumours,

Pituitary gland tumours, Polycythaemia vera, Primary CNS lymphoma, Primary mediastinal large B-cell lymphoma, Primary peritoneal cancer, Prostate cancer, Pseudomyxoma peritonei, Rare cancers, Rectal cancer, Recurrent melanoma, Rhabdomyosarcoma, Salivary gland cancer, Sarcoma, Secondary bone cancer, Secondary breast cancer, Secondary cancer, Secondary liver cancer, Secondary lung cancer, Skin cancer, Small bowel cancer, Small cell lung cancer, Small lymphocytic lymphoma, Soft tissue sarcoma, Spinal cord tumours, Splenic marginal zone lymphoma, Stomach cancer, Testicular cancer, Throat cancer, Thymus gland cancer, Thyroid cancer, Tongue cancer, Tracheal cancer. Triple negative breast cancer, Ureter and renal pelvis cancers, Vaginal cancer, Vulval cancer, Waldenström's macroglobulinaemia, Womb cancer.

By inhibiting PDGFR activation induced tumour cell proliferation and migration, EVs may inhibit both tumour growth and the formation of metastases.

The role of PDGFR inhibitors in the treatment of cancer is well-documented in the art. Our results indicate that EVs may be used similarly to PDGFR inhibitor anticancer drugs, ie. in the treatment or prevention of cancers. Cancer types wherein PDFR inhibitor drugs are clinically tested are e.g. glioblastoma multiforme, chordoma, meningeoma, dermatofibrosarcoma protuberance, gastrointestinal stromal tumour, soft tissue sarcoma, osteosarcoma, chronic myeloproliferative diseases, hypereosinophilic syndrome, prostate cancer, non-small cell lung cancer, neuroblastoma, liver cancer, colorectal cancer, mesothelioma, breast cancer, ovarian cancer, Leydig cell tumour. Known PDGFR targeting (inhibitor) drugs include Nilotinib, Dasatinib, Ponatinib, Sunitinib, Axitinib, Midostaurin, Pazopanib, Regorafenib, Sorafenib, Nintedanib, Lenvatinib, Masitinib, Crenolanib.

By inhibiting the PDGF induced activation (e.g. proliferation, migration, ECM production, tissue contraction) of fibroblasts (cancer associated fibroblasts) present in the tumour stroma, EVs may be used to enhance the effect of other anticancer drugs as well.

PDGF induces the contraction of fibroblasts present in the tumour stroma, which leads to the contraction of the tumour tissue of solid tumours. As a result, intratumoural pressure increases, reducing capillary flow thereby limiting the amount of anticancer drug reaching the tumour cells. EVs may be used to treat solid tumours by inhibiting the activity, in particular by inhibiting the PDGF induced contraction of fibroblasts present in the tumour stroma, selected from the group consisting of acoustic neuroma, melanoma, prostate cancer, anal cancer, anaplastic thyroid cancer, anorectal melanoma, astrocytoma, bile duct cancer (cholangiocarcinoma), bladder cancer, bone cancer, bowel cancer, brain cancer, breast cancer, breast cancer in men, solid cancer of unknown primary, cervical cancer, chondrosarcoma chordoma, colon cancer, craniopharyngioma, ductal carcinoma in situ, early (localised) prostate cancer, ependymoma, ewing sarcoma, eye cancer (ocular melanoma), fallopian tube cancer, gallbladder cancer, gastrointestinal stromal tumour, germ cell ovarian cancer, glioma, haemangioblastoma, head and neck cancer, inflammatory breast cancer, laryngeal (larynx) cancer, leiomyosarcoma, liver cancer, locally prostate cancer, lung cancer, secondary, medullary thyroid cancer, medulloblastoma, meningioma, mesothelioma, mouth cancer, nasal and sinus cancer, nasopharyngeal cancer, neuroendocrine tumours, non-small cell lung cancer, oesophageal cancer, oligodendroglioma, oropharyngeal cancer, osteosarcoma, ovarian cancer, paget's disease of the breast, pancreatic cancer, papillary and follicular thyroid cancer, parathyroid cancer, penile cancer, phyllodes tumours, pineal region tumours, pituitary gland tumours, primary peritoneal cancer, prostate cancer, pseudomyxoma peritonei, rectal cancer, recurrent melanoma, rhabdomyosarcoma, salivary gland cancer, sarcoma, secondary bone cancer, secondary breast cancer, secondary solid cancer, skin cancer, small bowel cancer, small cell lung cancer, soft tissue sarcoma, spinal cord tumours, stomach cancer, testicular cancer, throat cancer, thymus gland cancer, thyroid cancer, tongue cancer, tracheal cancer, triple negative breast cancer, ureter and renal pelvis cancers, vaginal cancer, vulval cancer, womb cancer.

Our findings that EVs inhibit the activation of TGF induced signalling pathways and PDGFB gene expression (see above) also indicate that EVs may be used in anticancer therapy. The role of TGFβ signalling pathway inhibitors in the treatment of cancer is well-documented in the art. Our results indicate that EVs may be used similarly to TGFβ inhibitor anticancer drugs, ie. in the treatment or prevention of cancer.

Clinical use of inhibitors of the TGFβ signalling pathways has already been tested in e.g. gastric cancer, non-small cell lung cancer, ovarian cancer, colon cancer, Ewing's sarcoma, Hepatocellular carcinoma, prostate cancer, uterus carcinoma. Known TGFβ targeting drugs include Vactosertib, Galunisertib, Fresolimumab, Bintrafusp alfa, Trabedersen, Belagenpumatucel-L, Luspatercept, Tasisulam, AVID200, LY3200882, A83-01, LY2109761, SB-431542.

Our results indicate that EVs may be used similarly to EGFR inhibitor anticancer drugs, ie. in the treatment or prevention of cancer.

Clinical use of inhibitors of the EGFR inhibitors has already been tested in e.g. acute myeloid leukemia, colorectal cancer, head and neck cancer, lung cancer, breast and pancreatic cancer. Known EGFR inhibitor drugs include erlotinib, osimetinib, neratinib, gefitinib, cetuximab, panitumumab, dacomitinib, lapatinib, necitumumab, mobocertinib, vandetanib, almonertinib, brigatinib, olmutinib, pyrotinib, afatinib, acomitinib, icotinib, simotinib. Our results demonstrate that EVs may be used in modulating, preferably inhibiting the activity of PDGF signalling pathways. Preferably, EVs are for use in modulating the activity of any one of the PDGFR related signal transduction molecules in Table 1. Preferably, EVs are for use in the prevention or treatment of a condition, disorder or disease associated with the activity of any one of the signal transduction molecules in Table 1.

Signal transduction molecules that might be affected by EVs are shown it Table 1.

| | |
|---|---|
| AKT1 | AKT serine/threonine kinase 1 |
| AKT2 | AKT serine/threonine kinase 2 |
| AKT3 | AKT serine/threonine kinase 3 |
| ARAF | A-Raf proto-oncogene. serine/threonine kinase |
| AXL | AXL receptor tyrosine kinase |
| BAD | BCL2 associated agonist of cell death |
| BAX | BCL2 associated X, apoptosis regulator |
| BCL2 | BCL2 apoptosis regulator |
| BCL2L1 | BCL2 like 1 |
| BCL2L11 | BCL2 like 11 |
| BIM | BCL2 like 11 |
| BRAF | B-Raf proto-oncogene, serine/threonine kinase |
| CCND1 | cyclin D1 |
| EGF | epidermal growth factor |
| EGFR | epidermal growth factor receptor |
| EIF4E | eukaryotic translation initiation factor 4E |
| EIF4E2 | eukaryotic translation initiation factor 4E family member 2 |
| EIF4EBP1 | eukaryotic translation initiation factor 4E binding protein 1 |
| ELK1 | ETS transcription factor ELK1 |
| ERBB2 | erb-b2 receptor tyrosine kinase 2 |
| ERBB3 | erb-b2 receptor tyrosine kinase 3 |
| ERK1 | mitogen-activated protein kinase 3 |
| FGF2 | fibroblast growth factor 2 |
| FGFR2 | fibroblast growth factor receptor 2 |
| FGFR3 | fibroblast growth factor receptor 3 |
| FOS | Fos proto-oncogene, AP-1 transcription factor subunit |
| FOXO3 | forkhead box O3 |
| GAB1 | GRB2 associated binding protein 1 |
| GAS6 | growth arrest specific 6 |
| GRB1 | phosphoinositide-3-kinase regulatory subunit 1 |
| GRB2 | growth factor receptor bound protein 2 |
| GRB2 | growth factor receptor bound protein 2 |
| GSK3B | glycogen synthase kinase 3 beta |
| HGF | hepatocyte growth factor |
| HRAS | HRas proto-oncogene, GTPase |
| HRAS | HRas proto-oncogene, GTPase |
| IGF1 | insulin like growth factor 1 |
| IGF1R | insulin like growth factor 1 receptor |
| IL6 | interleukin 6 |
| IL6R | interleukin 6 receptor |
| JAK1 | Janus kinase 1 |
| JAK2 | Janus kinase 2 |
| JNK1 | mitogen-activated protein kinase 8 |
| JNKK1 | mitogen-activated protein kinase kinase 4 |
| JUN | Jun proto-oncogene, AP-1 transcription factor subunit |
| KDR | kinase insert domain receptor |
| KRAS | KRAS proto-oncogene, GTPase |
| MAP2K1 | mitogen-activated protein kinase kinase 1 |
| MAP2K2 | mitogen-activated protein kinase kinase 2 |
| MAPK1 | mitogen-activated protein kinase 1 |
| MAPK3 | mitogen-activated protein kinase 3 |
| MEK1 | mitogen-activated protein kinase kinase 1 |
| MEKK | mitogen-activated protein kinase kinase kinase 1 |
| MET | MET proto-oncogene, receptor tyrosine kinase |
| MRAS | muscle RAS oncogene homolog |
| MTOR | mechanistic target of rapamycin kinase |
| MYC | MYC proto-oncogene, bHLH transcription factor |
| NF1 | neurofibromin 1 |
| NRAS | NRAS proto-oncogene, GTPase |
| NRG1 | neuregulin 1 |
| NRG2 | neuregulin 2 |
| PDGFA | platelet derived growth factor subunit A |
| PDGFB | platelet derived growth factor subunit B |
| PDGFC | platelet derived growth factor C |
| PDGFD | platelet derived growth factor D |
| PDGFRA | platelet derived growth factor receptor alpha |
| PDGFRB | platelet derived growth factor receptor beta |
| PDGFRB | platelet derived growth factor receptor beta |
| PDPK1 | 3-phosphoinositide dependent protein kinase 1 |
| PIK3CA | phosphatidylinositol-4,5-bisphosphate 3-kinase catalytic subunit alpha |
| PIK3CA | phosphatidylinositol-4,5-bisphosphate 3-kinase catalytic subunit alpha |
| PIK3CB | phosphatidylinositol-4,5-bisphosphate 3-kinase catalytic subunit beta |
| PIK3CD | phosphatidylinositol-4,5-bisphosphate 3-kinase catalytic subunit delta |
| PIK3R1 | phosphoinositide-3-kinase regulatory subunit 1 |
| PIK3R2 | phosphoinositide-3-kinase regulatory subunit 2 |
| PIK3R3 | phosphoinositide-3-kinase regulatory subunit 3 |
| PKR | eukaryotic translation initiation factor 2 alpha kinase 2 |
| PLCG1 | phospholipase C gamma 1 |
| PLCG1 | phospholipase C gamma 1 |
| PLCG2 | phospholipase C gamma 2 |
| PRKCA | protein kinase C alpha |
| PRKCA | protein kinase C alpha |
| PRKCB | protein kinase C beta |
| PRKCB | protein kinase C beta |
| PRKCG | protein kinase C gamma |
| PTEN | phosphatase and tensin homolog |
| RAF1 | Raf-1 proto-oncogene, serine/threonine kinase |
| RAF1 | Raf-1 proto-oncogene, serine/threonine kinase |
| RASA1 | RAS p21 protein activator 1 |
| RPS6 | ribosomal protein S6 |
| RPS6KB1 | ribosomal protein S6 kinase B1 |
| RPS6KB2 | ribosomal protein S6 kinase B2 |
| RRAS | RAS related |
| RRAS2 | RAS related 2 |
| SHC1 | SHC adaptor protein 1 |
| SHC1 | SHC adaptor protein 1 |
| SHC2 | SHC adaptor protein 2 |
| SHC3 | SHC adaptor protein 3 |
| SHC4 | SHC adaptor protein 4 |
| SOS1 | SOS Ras/Rac guanine nucleotide exchange factor 1 |
| SOS1 | SOS Ras/Rac guanine nucleotide exchange factor 1 |
| SOS2 | SOS Ras/Rho guanine nucleotide exchange factor 2 |
| SRC | SRC proto-oncogene, non-receptor tyrosine kinase |
| SRF | serum response factor |
| STAT1 | signal transducer and activator of transcription 1 |
| STAT3 | signal transducer and activator of transcription 3 |
| STAT3 | signal transducer and activator of transcription 3 |
| STAT5A | signal transducer and activator of transcription 5A |
| STAT5B | signal transducer and activator of transcription 5B |
| STAT6 | signal transducer and activator of transcription 6 |
| TGFA | transforming growth factor alpha |
| VEGFA | vascular endothelial growth factor A |

Our results demonstrate that EVs may be used in modulating, preferably inhibiting the activity of the EGFR related signalling pathways. Preferably, EVs are for use in modulating the activity of any one of the signal transduction molecules in Table 2. Preferably, EVs are for use in the prevention or treatment of a condition, disorder or disease associated with the activity of any one of the signal transduction molecules in Table 2. Signal transduction molecules that might be affected by EVs are shown in Table 2.

TABLE 2

| | |
|---|---|
| ABI1 | abl interactor 1 |
| ABL1 | ABL proto-oncogene 1, non-receptor tyrosine kinase |
| AKT1 | AKT serine/threonine kinase 1 |
| AP2A1 | adaptor related protein complex 2 subunit alpha 1 |
| AP2B1 | adaptor related protein complex 2 subunit beta 1 |
| AP2M1 | adaptor related protein complex 2 subunit mu 1 |
| AP2S1 | adaptor related protein complex 2 subunit sigma 1 |
| ARF6 | ADP ribosylation factor 6 |
| ARHGEF1 | Rho guanine nucleotide exchange factor 1 |
| ASAP1 | ArfGAP with SH3 domain. ankyrin repeat and PH domain 1 |
| ATF1 | activating transcription factor 1 |
| ATXN2 | ataxin 2 |
| AURKA | aurora kinase A |
| BCAR1 | BCAR1 scaffold protein, Cas family member |
| BRAF | B-Raf proto-oncogene, serine/threonine kinase |
| CAMK2A | calcium/calmodulin dependent protein kinase II alpha |
| CAV1 | caveolin 1 |
| CAV2 | caveolin 2 |
| CBL | Cbl proto-oncogene |
| CBLB | Cbl proto-oncogene B |
| CBLC | Cbl proto-oncogene C |
| CDC42 | cell division cycle 42 |
| CFL1 | cofilin 1 |
| COX2 | cytochrome c oxidase subunit II |
| CREB1 | CAMP responsive element binding protein 1 |
| CRK | CRK proto-oncogene, adaptor protein |
| CRKL | CRK like proto-oncogene, adaptor protein |
| CSK | C-terminal Src kinase |
| DNM1 | dynamin 1 |
| DOK2 | docking protein 2 |
| E2F1 | E2F transcription factor 1 |
| EGF | epidermal growth factor |
| EGFR | epidermal growth factor receptor |
| EIF4EBP1 | eukaryotic translation initiation factor 4E binding protein 1 |
| ELK1 | ETS transcription factor ELK1 |
| ELK4 | ETS transcription factor ELK4 |
| EPN1 | epsin 1 |
| EPS15 | epidermal growth factor receptor pathway substrate 15 |
| EPS15L1 | epidermal growth factor receptor pathway substrate 15 like 1 |
| EPS8 | epidermal growth factor receptor pathway substrate 8 |
| ERBB2 | erb-b2 receptor tyrosine kinase 2 |
| ERRFI1 | ERBB receptor feedback inhibitor 1 |
| FOS | Fos proto-oncogene, AP-1 transcription factor subunit |
| FOSB | FosB proto-oncogene, AP-1 transcription factor subunit |
| FOXO1 | forkhead box O1 |
| FOXO4 | forkhead box O4 |
| GAB1 | GRB2 associated binding protein 1 |
| GAB2 | GRB2 associated binding protein 2 |
| GJA1 | gap junction protein alpha 1 |
| GRB10 | growth factor receptor bound protein 10 |
| GRB2 | growth factor receptor bound protein 2 |
| HGS | hepatocyte growth factor-regulated tyrosine kinase substrate |
| HRAS | HRas proto-oncogene, GTPase |
| INPP5D | inositol polyphosphate-5-phosphatase D |
| INPPL1 | inositol polyphosphate phosphatase like 1 |
| IQGAP1 | IQ motif containing GTPase activating protein 1 |
| IQSEC1 | IQ motif and Sec7 domain ArfGEF 1 |
| ITCH | itchy E3 ubiquitin protein ligase |
| JAK1 | Janus kinase 1 |
| JAK2 | Janus kinase 2 |
| JUN | Jun proto-oncogene, AP-1 transcription factor subunit |
| JUND | JunD proto-oncogene, AP-1 transcription factor subunit |
| KRAS | KRAS proto-oncogene. GTPase |
| LIMK2 | LIM domain kinase 2 |
| MAP2K1 | mitogen-activated protein kinase kinase 1 |
| MAP2K2 | mitogen-activated protein kinase kinase 2 |
| MAP2K5 | mitogen-activated protein kinase kinase 5 |
| MAP3K1 | mitogen-activated protein kinase kinase kinase 1 |
| MAP3K2 | mitogen-activated protein kinase kinase kinase 2 |
| MAP3K3 | mitogen-activated protein kinase kinase kinase 3 |
| MAP3K4 | mitogen-activated protein kinase kinase kinase 4 |
| MAP4K1 | mitogen-activated protein kinase kinase kinase kinase 1 |
| MAPK1 | mitogen-activated protein kinase 1 |
| MAPK14 | mitogen-activated protein kinase 14 |
| MAPK3 | mitogen-activated protein kinase 3 |
| MAPK7 | mitogen-activated protein kinase 7 |
| MAPK8 | mitogen-activated protein kinase 8 |
| MAPK9 | mitogen-activated protein kinase 9 |
| MEF2A | myocyte enhancer factor 2A |
| MEF2C | myocyte enhancer factor 2C |
| MEF2D | myocyte enhancer factor 2D |
| MTOR | mechanistic target of rapamycin kinase |
| MYBL2 | MYB proto-oncogene like 2 |
| NCK1 | NCK adaptor protein 1 |
| NCK2 | NCK adaptor protein 2 |
| NCOA3 | nuclear receptor coactivator 3 |
| GRIM19 | NADH:ubiquinone oxidoreductase subunit A13 |
| NEDD4 | NEDD4 E3 ubiquitin protein ligase |
| NEDD8 | NEDD8 ubiquitin like modifier |
| NOS3 | nitric oxide synthase 3 |
| PAK1 | p21 (RAC1) activated kinase 1 |
| PCNA | proliferating cell nuclear antigen |
| PDPK1 | 3-phosphoinositide dependent protein kinase 1 |
| PEBP1 | phosphatidylethanolamine binding protein 1 |
| PIAS3 | protein inhibitor of activated STAT 3 |
| PIK3C2B | phosphatidylinositol-4-phosphate 3-kinase catalytic subunit type 2 beta |
| PIK3R1 | phosphoinositide-3-kinase regulatory subunit 1 |
| PIK3R2 | phosphoinositide-3-kinase regulatory subunit 2 |
| PLCE1 | phospholipase C epsilon 1 |
| PLCG1 | phospholipase C gamma 1 |
| PLD1 | phospholipase D1 |
| PLD2 | phospholipase D2 |
| PLSCR1 | phospholipid scramblase 1 |
| PRKCA | protein kinase C alpha |
| PRKCB | protein kinase C beta |
| PRKCD | protein kinase C delta |
| PRKCI | protein kinase C iota |
| PRKCZ | protein kinase C zeta |
| PTEN | phosphatase and tensin homolog |
| PTK2 | protein tyrosine kinase 2 |
| PTK2B | protein tyrosine kinase 2 beta |
| PTK6 | protein tyrosine kinase 6 |
| PTPN11 | protein tyrosine phosphatase non-receptor type 11 |
| PTPN12 | protein tyrosine phosphatase non-receptor type 12 |
| PTPN5 | protein tyrosine phosphatase non-receptor type 5 |
| PTPRR | protein tyrosine phosphatase receptor type R |
| PXN | paxillin |
| RAB5A | RAB5A. member RAS oncogene family |
| RAC1 | Rac family small GTPase 1 |
| RAF1 | Raf-1 proto-oncogene, serine/threonine kinase |
| RALA | RAS like proto-oncogene A |
| RALB | RAS like proto-oncogene B |
| RALBP1 | ralA binding protein 1 |
| RALGDS | ral guanine nucleotide dissociation stimulator |
| RAP1A | RAP1A, member of RAS oncogene family |
| RASA1 | RAS p21 protein activator 1 |
| REPS2 | RALBP1 associated Eps domain containing 2 |
| RICTOR | RPTOR independent companion of MTOR complex 2 |
| RIN1 | Ras and Rab interactor 1 |
| ROCK1 | Rho associated coiled-coil containing protein kinase 1 |
| RPS6KA1 | ribosomal protein S6 kinase A1 |
| RPS6KA2 | ribosomal protein S6 kinase A2 |
| RPS6KA3 | ribosomal protein S6 kinase A3 |
| RPS6KA5 | ribosomal protein S6 kinase A5 |
| RPS6KB1 | ribosomal protein S6 kinase B1 |
| SH2D2A | SH2 domain containing 2A |
| SH3GL2 | SH3 domain containing GRB2 like 2, endophilin A1 |
| SH3GL3 | SH3 domain containing GRB2 like 3, endophilin A3 |
| SH3KBP1 | SH3 domain containing kinase binding protein 1 |
| SHC1 | SHC adaptor protein 1 |
| SOS1 | SOS Ras/Rac guanine nucleotide exchange factor 1 |
| SOS2 | SOS Ras/Rho guanine nucleotide exchange factor 2 |
| SP1 | Sp1 transcription factor |
| SPRY2 | sprouty RTK signalling antagonist 2 |
| SRC | SRC proto-oncogene. non-receptor tyrosine kinase |
| STAM2 | signal transducing adaptor molecule |
| STAM | signal transducing adaptor molecule 2 |
| STAMBP | STAM binding protein |
| STAT1 | signal transducer and activator of transcription 1 |
| STAT3 | signal transducer and activator of transcription 3 |
| STAT5A | signal transducer and activator of transcription 5A |
| STAT5B | signal transducer and activator of transcription 5B |
| STMN1 | stathmin 1 |
| STXBP1 | syntaxin binding protein 1 |
| SYNJ1 | synaptojanin 1 |
| TNK2 | tyrosine kinase non receptor 2 |
| TWIST1 | twist family bHLH transcription factor 1 |
| USP6NL | USP6 N-terminal like |
| USP8 | ubiquitin specific peptidase 8 |
| VAV1 | vav guanine nucleotide exchange factor 1 |
| VAV2 | vav guanine nucleotide exchange factor 2 |
| VAV3 | vav guanine nucleotide exchange factor 3 |

Our results demonstrate that EVs may be used in modulating, preferably inhibiting the activity of the TGFR related signalling pathways. Preferably, EVs are for use in modulating the activity of any one of the signal transduction molecules in Table 3. Preferably, EVs are for use in the prevention or treatment of a condition, disorder or disease associated with the activity of any one of the signal transduction molecules in Table 3. Signal transduction molecules that might be affected by EVs are shown it Table 3.

TABLE 3

| | |
|---|---|
| AKT1 | AKT serine/threonine kinase 1 |
| APP | amyloid beta precursor protein |
| ATF2 | activating transcription factor 2 |
| ATF3 | activating transcription factor 3 |
| AXIN1 | axin 1 |
| BCAR1 | BCAR1 scaffold protein, Cas family member |
| BMP4 | bone morphogenetic protein 4 |
| BTRC | beta-transducin repeat containing E3 ubiquitin protein ligase |
| CAV1 | caveolin 1 |
| CCNB2 | cyclin B2 |
| CCND1 | cyclin D1 |
| CDC42 | cell division cycle 42 |
| CDK1 | cyclin dependent kinase 1 |
| CDKNIA | cyclin dependent kinase inhibitor 1A |
| CDKN2B | cyclin dependent kinase inhibitor 2B |
| CITED1 | Cbp/p300 interacting transactivator with Glu/Asp |
| | rich carboxy-terminal domain 1 |
| COL1A2 | collagen type I alpha 2 chain |
| COPS5 | COP9 signalosome subunit 5 |
| CREBBP | CREB binding protein |
| CREBBP | CREB binding protein |
| CTNNB1 | catenin beta 1 |
| CUL1 | cullin 1 |
| DAB2 | DAB adaptor protein 2 |
| DCP1A | decapping mRNA 1A |
| E2F4 | E2F transcription factor 4 |
| E2F5 | E2F transcription factor 5 |
| EGF | epidermal growth factor |
| EID2 | EP300 interacting inhibitor of differentiation 2 |
| ENG | endoglin |
| EP300 | E1A binding protein p300 |
| EP300 | E1A binding protein p300 |
| ETS1 | ETS proto-oncogene 1, transcription factor |
| FKBP1A | FKBP prolyl isomerase 1A |
| FN1 | fibronectin 1 |
| FOS | Fos proto-oncogene, AP-1 transcription factor subunit |
| FOSB | FosB proto-oncogene, AP-1 transcription factor subunit |
| FOXH1 | forkhead box H1 |
| FOXH1 | forkhead box H1 |
| FOXP3 | forkhead box P3 |
| FST | follistatin |
| GRB2 | growth factor receptor bound protein 2 |
| HDAC1 | histone deacetylase 1 |
| HGS | hepatocyte growth factor-regulated tyrosine kinase substrate |
| HRAS | HRas proto-oncogene, GTPase |
| IFNG | interferon gamma |
| INHBA | inhibin subunit beta A |
| ITCH | itchy E3 ubiquitin protein ligase |
| ITGA2 | integrin subunit alpha 2 |
| ITGB1 | integrin subunit beta 1 |
| ITGB3 | integrin subunit beta 3 |
| ITGB4 | integrin subunit beta 4 |
| ITGB6 | integrin subunit beta 6 |
| JAK1 | Janus kinase 1 |
| JUN | Jun proto-oncogene, AP-1 transcription factor subunit |
| JUNB | JunB proto-oncogene, AP-1 transcription factor subunit |
| JUND | JunD proto-oncogene, AP-1 transcription factor subunit |
| KLF10 | Kruppel like factor 10 |
| KLF11 | Kruppel like factor 11 |
| KLF6 | Kruppel like factor 6 |
| LEF1 | lymphoid enhancer binding factor 1 |
| LEFTY1 | left-right determination factor 1 |
| LEFTY2 | left-right determination factor 2 |
| LIF | LIF interleukin 6 family cytokine |
| LIMK2 | LIM domain kinase 2 |
| LTBP1 | latent transforming growth factor beta binding protein 1 |
| MAP2K1 | mitogen-activated protein kinase kinase 1 |
| MAP2K2 | mitogen-activated protein kinase kinase 2 |
| MAP2K3 | mitogen-activated protein kinase kinase 3 |
| MAP2K4 | mitogen-activated protein kinase kinase 4 |
| MAP2K6 | mitogen-activated protein kinase kinase 6 |
| MAP3K7 | mitogen-activated protein kinase kinase kinase 7 |
| MAP4K1 | mitogen-activated protein kinase kinase kinase kinase 1 |
| MAPK1 | mitogen-activated protein kinase 14 |
| MAPK14 | mitogen-activated protein kinase 1 |
| MAPK3 | mitogen-activated protein kinase 3 |
| MAPK3 | mitogen-activated protein kinase 3 |
| MAPK8 | mitogen-activated protein kinase 8 |
| MAPK9 | mitogen-activated protein kinase 9 |
| MAPK9 | mitogen-activated protein kinase 9 |
| MEF2A | myocyte enhancer factor 2A |
| MEF2C | myocyte enhancer factor 2C |
| MET | MET proto-oncogene, receptor tyrosine kinase |
| MIR302A | microRNA 302a |
| MMP1 | matrix metallopeptidase 12 |
| MMP12 | matrix metallopeptidase 1 |
| MTOR | mechanistic target of rapamycin kinase |
| MYC | MYC proto-oncogene, bHLH transcription factor |
| NEDD4L | NEDD4 like E3 ubiquitin protein ligase |
| NEDD9 | neural precursor cell expressed, developmentally down-regulated 9 |
| NFKB1 | nuclear factor kappa B subunit 1 |
| NOG | noggin |
| NUP153 | nucleoporin 153 |
| NUP214 | nucleoporin 214 |
| PAK2 | p21 (RAC1) activated kinase 2 |
| PARD6A | par-6 family cell polarity regulator alpha |
| RUNX2 | RUNX family transcription factor 2 |
| RUNX3 | RUNX family transcription factor 3 |
| SERPINE1 | serpin family E member 1 |
| SKI | SKI proto-oncogene |
| SKIL | SKI like proto-oncogene |
| SMAD1 | SMAD family member 1 |
| SMAD2 | SMAD family member 2 |
| SMAD3 | SMAD family member 3 |
| SMAD4 | SMAD family member 4 |
| SMAD5 | SMAD family member 5 |
| SMAD6 | SMAD family member 6 |
| SMAD7 | SMAD family member 7 |
| SMAD9 | SMAD family member 9 |
| SPP1 | secreted phosphoprotein 1 |
| STAT1 | signal transducer and activator of transcription 1 |
| STAT3 | signal transducer and activator of transcription 3 |
| TFE3 | transcription factor binding to IGHM enhancer 3 |
| TGFB1 | transforming growth factor beta 1 |
| TGFBR1 | transforming growth factor beta receptor 1 |
| TGFBR2 | transforming growth factor beta receptor 2 |
| TGFBR3 | transforming growth factor beta receptor 3 |
| TGIF | TGFB induced factor homeobox 1 |
| THBS1 | thrombospondin 1 |
| TNF | tumour necrosis factor |
| WNT1 | Wnt family member 1 |
| ZFHX1B | zinc finger E-box binding homeobox 2 |
| ZFYVE9 | zinc finger FYVE-type containing 9 |
| ZNF423 | zinc finger protein 423 |

Our results demonstrate that EVs may be used in modulating, preferably inhibiting the activity of the common elements of PDGFR and EGFR signalling pathways. Preferably, EVs are for use in modulating the activity of any one of the signal transduction molecules in Table 4. Preferably, EVs are for use in the prevention or treatment of a condition, disorder or disease associated with the activity of any one of the signal transduction molecules in Table 4.

Signal transduction molecules that might be affected by EVs are shown in Table 4.

TABLE 4

| | |
|---|---|
| BRAF | B-Raf proto-oncogene, serine/threonine kinase |
| EGFR | epidermal growth factor receptor |
| EIF4EBP1 | eukaryotic translation initiation factor 4E binding protein 1 |
| ELK1 | ETS transcription factor ELK1 |
| ERBB2 | erb-b2 receptor tyrosine kinase 2 |
| GAB1 | GRB2 associated binding protein 1 |
| JAK2 | Janus kinase 2 |
| KRAS | KRAS proto-oncogene, GTPase |
| PDPK1 | 3-phosphoinositide dependent protein kinase 1 |
| PIK3R1 | phosphoinositide-3-kinase regulatory subunit 1 |
| PIK3R2 | phosphoinositide-3-kinase regulatory subunit 2 |
| PLCG1 | phospholipase C gamma 1 |
| PRKCA | protein kinase C alpha |
| PRKCB | protein kinase C beta |
| PTEN | phosphatase and tensin homolog |
| RAF1 | Raf-1 proto-oncogene, serine/threonine kinase |
| RASA1 | RAS p21 protein activator 1 |
| RPS6KB1 | ribosomal protein S6 kinase B1 |
| SHC1 | SHC adaptor protein 1 |
| SOS1 | SOS Ras/Rac guanine nucleotide exchange factor 1 |
| SOS2 | SOS Ras/Rho guanine nucleotide exchange factor 2 |
| SRC | SRC proto-oncogene, non-receptor tyrosine kinase |
| STAT5A | signal transducer and activator of transcription 5A |
| STAT5B | signal transducer and activator of transcription 5B |
| AKT1 | AKT serine/threonine kinase 1 |
| EGF | epidermal growth factor |
| FOS | Fos proto-oncogene, AP-1 transcription factor subunit |
| GRB2 | growth factor receptor bound protein 2 |
| HRAS | HRas proto-oncogene, GTPase |
| JAK1 | Janus kinase 1 |
| JUN | Jun proto-oncogene, AP-1 transcription factor subunit |
| MAP2K1 | mitogen-activated protein kinase kinase 1 |
| MAP2K2 | mitogen-activated protein kinase kinase 2 |
| MAPK1 | mitogen-activated protein kinase 1 |
| MAPK3 | mitogen-activated protein kinase 3 |
| MTOR | mechanistic target of rapamycin kinase |
| STAT1 | signal transducer and activator of transcription 1 |
| STAT3 | signal transducer and activator of transcription 3 |

Our results demonstrate that EVs may be used in modulating, preferably inhibiting the activity of the common elements of TGF and PDGF induced signalling pathways. Preferably, EVs are for use in modulating the activity of any one of the signal transduction molecules in Table 5. Preferably, EVs are for use in the prevention or treatment of a condition, disorder or disease associated with the activity of any one of the signal transduction molecules in Table 5.

Signal transduction molecules that might be affected by EVs are shown in Table 5.

TABLE 5

| | |
|---|---|
| CCND1 | cyclin D1 |
| MET | MET proto-oncogene, receptor tyrosine kinase |
| MYC | MYC proto-oncogene, bHLH transcription factor |
| AKT1 | AKT serine/threonine kinase 1 |
| EGF | epidermal growth factor |
| FOS | Fos proto-oncogene, AP-1 transcription factor subunit |
| GRB2 | growth factor receptor bound protein 2 |
| HRAS | HRas proto-oncogene, GTPase |
| JAK1 | Janus kinase 1 |
| JUN | Jun proto-oncogene, AP-1 transcription factor subunit |
| MAP2K1 | mitogen-activated protein kinase kinase 1 |
| MAP2K2 | mitogen-activated protein kinase kinase 2 |
| MAPK1 | mitogen-activated protein kinase 1 |
| MAPK3 | mitogen-activated protein kinase 3 |
| MTOR | mechanistic target of rapamycin kinase |
| STAT1 | signal transducer and activator of transcription 1 |
| STAT3 | signal transducer and activator of transcription 3 |

Our results demonstrate that EVs may be used in modulating, preferably inhibiting the activity of the common elements of TGF and EGF induced signalling pathways. Preferably, EVs are for use in modulating the activity of any one of the signal transduction molecules in Table 6. Preferably, EVs are for use in the prevention or treatment of a condition, disorder or disease associated with the activity of any one of the signal transduction molecules in Table 6.

Signal transduction molecules that might be affected by EVs are shown in Table 6.

TABLE 6

| | |
|---|---|
| BCAR1 | BCAR1 scaffold protein. Cas family member |
| CAV1 | caveolin 1 |
| CDC42 | cell division cycle 42 |
| FOSB | FosB proto-oncogene, AP-1 transcription factor |
| | subunit |
| HGS | hepatocyte growth factor-regulated tyrosine kinase |
| | substrate |
| ITCH | itchy E3 ubiquitin protein ligase |
| JUND | JunD proto-oncogene, AP-1 transcription factor |
| | subunit |
| LIMK2 | LIM domain kinase 2 |
| MAP4K1 | mitogen-activated protein kinase kinase |
| | kinase kinase 1 |
| MAPK14 | mitogen-activated protein kinase 14 |
| MAPK8 | mitogen-activated protein kinase 8 |
| MAPK9 | mitogen-activated protein kinase 9 |
| MEF2A | myocyte enhancer factor 2A |
| MEF2C | myocyte enhancer factor 2C |
| AKT1 | AKT serine/threonine kinase 1 |
| EGF | epidermal growth factor |
| FOS | Fos proto-oncogene, AP-1 transcription factor subunit |
| GRB2 | growth factor receptor bound protein 2 |
| HRAS | HRas proto-oncogene, GTPase |
| JAK1 | Janus kinase 1 |
| JUN | Jun proto-oncogene, AP-1 transcription factor subunit |
| MAP2K1 | mitogen-activated protein kinase kinase 1 |
| MAP2K2 | mitogen-activated protein kinase kinase 2 |
| MAPK1 | mitogen-activated protein kinase 1 |
| MAPK3 | mitogen-activated protein kinase 3 |
| MTOR | mechanistic target of rapamycin kinase |
| STAT1 | signal transducer and activator of transcription 1 |
| STAT3 | signal transducer and activator of transcription 3 |

Our results demonstrate that EVs may be used in modulating, preferably inhibiting the activity of the TGF and EGFR and PDGF induced signalling pathways, preferably inhibiting the activity of the common elements of the TGF and EGFR and PDGF induced signalling pathways). Preferably, EVs are for use in modulating the activity of any one of the signal transduction molecules in Table 7. Preferably, EVs are for use in the prevention or treatment of a condition, disorder or disease associated with the activity of any one of the signal transduction molecules in Table 7.

Signal transduction molecules that might be affected by EVs are shown in Table 7.

TABLE 7

| | |
|---|---|
| AKT1 | AKT serine/threonine kinase 1 |
| EGF | epidermal growth factor |
| FOS | Fos proto-oncogene, AP-1 transcription factor subunit |
| GRB2 | growth factor receptor bound protein 2 |
| HRAS | HRas proto-oncogene, GTPase |
| JAK1 | Janus kinase 1 |
| JUN | Jun proto-oncogene, AP-1 transcription factor subunit |
| MAP2K1 | mitogen-activated protein kinase kinase 1 |
| MAP2K2 | mitogen-activated protein kinase kinase 2 |
| MAPK1 | mitogen-activated protein kinase 1 |
| MAPK3 | mitogen-activated protein kinase 3 |
| MTOR | mechanistic target of rapamycin kinase |
| STAT1 | signal transducer and activator of transcription 1 |
| STAT3 | signal transducer and activator of transcription 3 |

Our results demonstrate that EVs may be used in modulating IL-10 signalling through a receptor complex consisting of two IL-10 receptor-1 and two IL-10 receptor-2 proteins (Berman R M, Suzuki T, Tahara H, Robbins P D, Narula S K, Lotze M T (July 1996). “Systemic administration of cellular IL-10 induces an effective, specific, and long-lived immune response against established tumours in mice”. Journal of Immunology. 157 (1): 231-8. PMID 8683120). IL-10 has pleiotropic effects in immunoregulation and inflammation. Indeed, it has been sown to downregulate the expression of Th1 cytokines, MHC class II antigens, and co-stimulatory molecules on macrophages. Moreover it enhances B cell proliferation, survival, and antibody production. IL-10 can block NF-κB activity, and is involved in the regulation of the JAK-STAT signalling pathway. IL-10 also leads to control of primary tumour growth and decreased metastatic burden (Zheng L M, Ojcius D M, Garaud F, Roth C, Maxwell E, Li Z, Rong H, Chen J, Wang X Y, Catino J J, King I (August 1996). “Interleukin-10 inhibits tumour metastasis through an NK cell-dependent mechanism”. The Journal of Experimental Medicine. 184 (2): 579-84. doi: 10.1084/jem.184.2.579. PMC 2192723. PMID 8760811; Fujii S, Shimizu K, Shimizu T, Lotze M T (October 2001). “Interleukin-10 promotes the maintenance of antitumour CD8 (+) T-cell effector function in situ”. Blood. 98 (7): 2143-51. doi: 10.1182/blood.v98.7.2143. PMID 11568001; Berman R M, Suzuki T, Tahara H, Robbins P D, Narula S K, Lotze M T (July 1996). “Systemic administration of cellular IL-10 induces an effective, specific, and long-lived immune response against established tumours in mice”. Journal of Immunology. 157 (1): 231-8. PMID 8683120).

Endogenous metabolites, such as MGO, Glyoxal, 3,4 DGE, HMF etc, generated by the oxidation of glucose and other saccharides, reacting with proteins, lipides or nucleic acids form advanced glycation end products (AGEs). Accumulation of AGEs and their binding with their receptors promote oxidative stress and inflammation. Oxidative stress can disturb intracellular signals to become pathological states, particularly insulin-mediated metabolic responses and insulin resistance (Shen C Y, Lu C H, Wu C H, Li K J, Kuo Y M, Hsieh S C, Yu C L. The Development of Maillard Reaction, and Advanced Glycation End Product (AGE)-Receptor for AGE (RAGE) Signalling Inhibitors as Novel Therapeutic Strategies for Patients with AGE-Related Diseases. Molecules. 2020 Nov. 27; 25 (23): 5591. doi: 10.3390/molecules25235591. PMID: 33261212; PMCID: PMC7729569). Protein glycation and formation of AGEs play an important role in the pathogenesis of diabetic complications like retinopathy, nephropathy, neuropathy, cardiomyopathy (Singh V P, Bali A, Singh N, Jaggi A S. Advanced glycation end products and diabetic complications. Korean J Physiol Pharmacol. 2014; 18 (1): 1-14. doi: 10.4196/kjpp.2014.18.1.1). AGEs have a pathogenetic role in the development and progression of different oxidative-based diseases including diabetes (Vistoli G, De Maddis D, Cipak A, Zarkovic N, Carini M, Aldini G. Advanced glycoxidation and lipoxidation end products (AGEs and ALEs): an overview of their mechanisms of formation. Free Radic Res. 2013 August; 47 Suppl 1:3-27. doi: 10.3109/10715762.2013.815348. PMID: 23767955.). Based on our results (FIG. 41-44) EVs are suitable for use in the prevention or treatment of disorders or diseases associated with impaired glucose metabolism, such as insulin resistance, diabetes, diabetic complications or disorders or diseases associated with diabetes and in the prevention or treatment of conditions, disorders or diseases associated with the formation of MGO, such as insulin resistance, type I diabetes mellitus, type II diabetes mellitus, diabetic nephropathy, diabetic ulcer, diabetic retinopathy, macular degeneration, myocardial infarction, hypertension, arteriosclerosis, atherosclerosis, venosclerosis, irritable Bowel Syndrome, rheumatoid arthritis, osteoarthritis, Parkinson's diseases, Alzheimer's diseases, dementia, depression, anxiety disorders, schizophrenia and various cancers.

Our results indicate that EVs may be used in therapy similarly to PDGFR, EGFR or TGFR inhibitors or agents inhibiting any of the signalling pathways activated by PDGF, EGF or TGF or preferably inhibiting the activity of the common elements of the TGF and EGFR induced, or the TGF and PDGF induced, or the EGFR and PDGF induced, preferably the TGF and EGFR and PDGF induced signalling pathways. Accordingly, EVs may be used in the therapy of conditions, disorders or diseases wherein a PDGFR, EGFR or TGFR inhibitor or an agent inhibiting any of the signalling pathways mediated by PDGF, EGF or TGF is useful.

Our results also indicate that EVs may be used in the non-medical (e.g., cosmetic) treatment of fibroproliferative or fibrotic conditions, such as age-related tissue fibrosis. Therefore, food products, cosmetic compositions comprising or essentially consisting of algal EVs for the treatment or inhibition of fibroproliferative or fibrotic conditions are also provided.

Formulation of EVs into composition may be carried out as disclosed herein upon preparation of EVs. In fact providing EVs in an appropriate amount and concentration and in any appropriate medium is described in the Examples. EVs are usually stable and the description provides methods for their culture, concentration, pelleting and change of medium. Moreover, providing EVs in the form of compositions and developing them into medicaments are known in the art.

The following publications provide examples for developing EVs into formulations. The skilled person will understand that formulation methods and compositions developed for EVs can be applied, mutatis mutandis to algal EVs as well.

Karamanidou, T.; Tsouknidas, A. Plant-Derived Extracellular Vesicles as Therapeutic Nanocarriers. Int. J. Mol. Sci. 2022, 23, 191. https://doi.org/10.3390/ijms23010191

Klyachko, Natalia L et al. “Extracellular Vesicle-Based Therapeutics: Preclinical and Clinical Investigations.” Pharmaceutics vol. 12,12 1171. 1 Dec. 2020, doi: 10.3390/pharmaceutics12121171

Wiklander, Oscar P B et al. “Advances in therapeutic applications of extracellular vesicles.” Science translational medicine vol. 11,492 (2019): eaav8521. doi: 10.1126/scitranslmed.aav8521

Claridge B et al. Development of Extracellular Vesicle Therapeutics: Challenges, Considerations, and OpportunitiesCell Dev. Biol., 20 Sep. 2021 | https://doi.org/10.3389/fcell.2021.734720

Ana Rita Marques Pereira Trindade, “FORMULATION STRATEGIES FOR EXTRACELLULAR VESICLE DELIVERY” PHD thesis, 2018.

### Examples—Methods

Algae Cultivation: Chlorella sp. MACC-360, Parachlorella kessleri were grown in TAP media (UTEX, USA), MACC-215 (Chlamydomonas reinhardtii), MACC-1 (Chlorella vulgaris), MACC-3 (Chlorella pyrenoidosa), MACC-1023 (Hormidiospora verrucosa), MACC-21 (Zygnema peliosporum), MACC-1022 (Vischeria polyphem), MACC-888 (Microcystis aeruginosa) and MACC-90 (Haematococcus lacustris) were grown in Bristol (UTEX, USA) media, MACC-612 (Nostoc linckia), and MACC-918 (Spirogyra sp) and MACC-42 (Arthronema africanum) were grown in Z-8 media (http://www.sccap.dk/media/freshwater/7.asp), MACC-908 (Spirulina platensis), MACC-909 (Spirulina maxima) were grown in Spirulina media (UTEX, USA), and MACC-115 (Synechococcus sp) and MACC-1042 (Klebsormidium nitens) were grown in BG-11 (N) media (UTEX, USA) in 250 ml Erlenmeyer flasks on an orbital shaker set at 150 rpm at room temperature. Unless otherwise indicated, autotrophic cultivation was provided by optimized conditions, ensuring 12 hours-day/12 hours-night photoperiods using white light bulb until cell count reached approximately 7×10\hat{ }7 cell/ml. In case of heterotrophic cultivation of Chlorella sp. MACC-360 was grown under dark conditions in flask, using RPMI 1640 medium (ATCC, USA) to ensure the sufficient source of nutrients. Algal strains were kindly provided by Molnár Zoltán (Mosonmagyaróvár Algal Culture Collection, Mosonmagyaróvár, Hungary). Chlorella pyrenoidosa (Hangzhou Natur Foods Co., China), Spirulina platensis (Hangzhou Natur Foods Co., China), Aschophyllum nodosum (Kelp, https://www.futunatura.hu), Palmaria palmate (Dulse, https://www.futunatura.hu), Tetraselmis chui (https://www.allmashop.com/produto/tetraselmis-chui-em-po), Aphanizomenon flos-aquae (https://www.futunatura.hu) were purchased as edible powder and rehydrated in phosphate buffered saline (PBS) at 20 mg/ml concentration for 1 hour at room temperature under gently agitation.

Extracellular vesicle (EV) isolation: To remove microbiological contaminants and cell debris, samples were centrifuged for 20 minutes at 2000 rpm, then the supernatants were filtrated with 0.22 μm pore size filter (Millipore Express® PLUS, Merck, Germany). Subsequently the EV content of the given sample was enriched and/or EVs were isolated by several ways (summarized in Table 8).
(1) Samples were ultrafiltered and concentrated at by tangential flow filtration, using TFF-easy filters (Hansa BioMed Life Sciences, BIOCENTER Laboratory Supplier Ltd, Hungary).
(2) Samples were ultrafiltered and concentrated by Amicon® Ultra-15 (100 kDa MWCO filters, Millipore Express® PLUS, Merck, Germany).
(3) Samples were ultracentrifuged by Beckman L7-55 Ultracentrifuge using Type 70.1 Ti Fixed-Angle Titanium Rotor (Beckman Coulter, USA) and 10 ml, Open-Top Thickwall Polycarbonate Tubes (Backman Coulter, USA) at 4° C. for 1 hour.
(4) Samples were dialyzed using SnakeSkin™ Dialysis Tubing (3.5K MWCO, ThermoFisher Scientific, USA) and 20% poly(ethylene glycol) solution (20K MW, Merck, Germany) for 24 hours.
(5) Samples were extruded after multiple steps of sonication (Kerry Steel Probe Sonicator Ultrasonic, immersed in liquid) or that of tissue homogenization in 2 ml Potter-Elvehjem Tissue Grinder in case of Spirulina platensis powder and freeze-thaw cycle (−80-60° C.) by LIPEX® Extruder (Evonic Industries, Germany) with polycarbonate membrane filters (Whatman, UK) at 60° C.
(6) A part of the isolated EV samples from (1), (2), (4) and (5) steps were further purified by size-exclusion chromatography (SEC) on IZON qEVoriginal/70 nm pore size columns (IZON, USA).

Unless otherwise indicated, EVs isolated from Chlorella sp. MACC-360 by TFF (1)+SEC (6) were used during the in vitro experiments.

TABLE 8
EV isolation methods from various algae sources.

| Species | Source | EV isolation techniques |
|---|---|---|
| Chlorella sp. MACC-360 | culture | TFF (1) |
| | | TFF (1) + SEC (6) |
| | | AMICON (2) |
| | | AMICON (2) + SEC (6) |
| | | UC (3) |
| | | DIAL (4) |
| | | DIAL (4) + SEC (6) |
| | | EXTR (5) |
| | | EXTR (5) + SEC (6) |
| Parachlorella kessleri | culture | TFF (1) + SEC (6) |
| Chlamydomonas | culture | TFF (1) + SEC (6) |
| reinhardtii | | |
| (MACC 215) | | |
| Chlorella vulgaris | culture | TFF (1) + SEC (6) |
| (MACC-1) | | |
| Chlorella pyrenoidosa | culture | TFF (1) + SEC (6) |
| (MACC-3) | | |
| Hormidiospora verrucosa | culture | TFF (1) + SEC (6) |
| (MACC-1023) | | |
| Zygnema peliosporum | culture | TFF (1) + SEC (6) |
| (MACC-21) | | |
| Vischeria polyphem | culture | TFF (1) + SEC (6) |
| (MACC-1022) | | |
| Microcystis aeruginosa | culture | TFF (1) + SEC (6) |
| (MACC-888) | | |
| Haematococcus lacustris | culture | TFF (1) + SEC (6) |
| (MACC-90) | | |
| Nostoc linckia (MACC-612) | culture | TFF (1) + SEC (6) |
| Spirogyra sp | culture | TFF (1) + SEC (6) |
| (MACC-918) | | |
| Arthronema africanum | culture | TFF (1) + SEC (6) |
| (MACC-42) | | |
| Spirulina platensis | culture | TFF (1) + SEC (6) |
| (MACC-908) | | |
| Spirulina maxima | culture | TFF (1) + SEC (6) |
| (MACC-909) | | |
| Synechococcus sp (MACC-115) | culture | TFF (1) + SEC (6) |
| Klebsormidium nitens | culture | TFF (1) + SEC (6) |
| (MACC-1042) | | |
| Chlorella pyrenoidosa | powder | AMICON (2) + SEC (6) |
| Spirulina platensis | powder | AMICON (2) + SEC (6) |
| Aschophyllum nodosum | powder | AMICON (2) + SEC (6) |
| Palmaria palmata | powder | AMICON (2) + SEC (6) |
| Tetraselmis chui | powder | AMICON (2) + SEC (6) |
| Aphanizomenon flos-aquae | powder | AMICON (2) + SEC (6) |

Fractions were collected and analysed by the following methods according to the ISEV (International Society for Extracellular Vesicles) recommendations to demonstrate and validate these samples EV's content.

Protein content, spectrophotometry: Protein content of samples was determined by NanoDrop ND-100 spectrophotometer (BCM, USA) based on their absorbance at 280 nm. Protein concentrations above 100 μg/ml predicted the success of EV isolation. In an alternative method, protein content of EV samples was determined by Qubit™ Protein Assay Kit (Q33211, Thermofisher Scientific, USA) following the manufacturer's instruction. Microfluidic resistive pulse sensing (MRPS): Microfluidic resistive pulse sensing (MRPS) is a novel non-optical method utilizing the Coulter principle for the size and concentration determination of various nano- and microparticles. MRPS measurements were performed with a nCS1 instrument (Spectradyne LLC, USA). The samples were diluted 10-fold with bovine serum albumin (BSA, Merck Kft, Hungary) solution at 1 mg/ml in PBS buffer (Merck Kft, Hungary) according to the manufacturer's instructions. Measurements were performed using factory calibrated TS-400, TS-900, and TS-2000 cartridges, which covers a measurement range from 65 nm to 2000 nm.

Nanoparticle tracking analysis (NTA): The size distribution, median size, and particle concentration of EVs were also determined using NTA. The measurement was performed with a ZetaView PMX-120 (Particle Metrix GmbH, Meerbusch, Germany) using Zeta VIEW software.

Dynamic light scattering (DLS): Hydrodynamic diameter of EVs were measured by a W130i dynamic light scattering (DLS) instrument (AvidNano, UK). Low-volume disposable plastic cuvettes were used for the DLS measurements (UVette; Eppendorf Austria, Austria), and data evaluation was performed with iSize 3.0 software (AvidNano).

Fourier-transform infrared spectroscopy (FTIR): Fourier-transform infrared spectroscopy (FTIR) was used to characterize the protein and lipid content of EVs. FTIR measurements were carried using a Varian 2000 spectrometer (Scimitar Series, USA), fitted with a diamond attenuated total reflection cell (‘Golden Gate’ single reflection ATR unit, Specac, United Kingdom). Approximately 5 μl of the sample was pipetted onto the diamond ATR surface and a thin dry film was obtained by slowly evaporating the solvent under ambient conditions (approx. 10 min). Typically, 64 scans were collected at a nominal resolution of 2 cm⁻¹. ATR correction, buffer background spectral subtraction and other spectral evaluations were performed with the Grams/32 software package (Galactic Inc., USA).

Freeze-fracture combined transmission electron microscopy (FF-TEM): Freeze-fracture combined transmission electron microscopy (FF-TEM) was used to reveal the morphology of EVs. The EV sample was mixed with glycerol (Sigma-Aldrich, Hungary), which is used as cryoprotectant at 3:1 sample-to-glycerol volume ratio. Approx. 2 μl vesicle sample was pipetted onto a gold sample holder and frozen by placing it immediately into partially solidified Freon for 20 seconds. Fracturing was performed at −100° C. in a Balzers freeze-fracture device (Balzers BAF 400D, Balzers A G, Liechtenstein). The replicas of the fractured surfaces were made by platinum-carbon evaporation and then cleaned with a water solution of surfactant and washed with distilled water. The platinum-carbon replicas were placed on 200 mesh copper grids and examined in a MORGAGNI 268D (FEI, The Netherlands) transmission electron microscope.

Lyophilisation: Peritoneal dialysis (PD) effluents (PDE) and EV samples were snap-frozen to the wall of a 50 ml Falcon centrifuge tube (PDE) or a 2 ml glass vial (EV samples) by spinning in liquid nitrogen in order to increase drying surface. Freeze-drying and setup of lyophilisation protocol was performed with a Scan Vac CoolSafe Touch Superior device (LaboGene A/S, Allerod, Denmark).

Human samples: Peritoneal dialysis (PD) effluents (PDE) were taken from patient receiving PD at the 1st Department of Paediatrics, Semmelweis University, Budapest, Hungary.

Peritoneal samples were collected at the time of the first insertion of Tenckhoff peritoneal catheter and at the time of PD catheter removal at the 1st Department of Paediatrics, Semmelweis University, Budapest, Hungary (loss of ultrafiltration capacity of the peritoneal membrane) (31224-5/2017/EKU).

Colon samples were taken at the 1st Department of Paediatrics, Semmelweis University Budapest, Hungary during routine endoscopy and the residues of these samples were used for our work (19048-4/2018/EKU).

Skin samples were kindly provided by Department of Dermatology, Venereology and Dermatooncology, Semmelweis University (Kende Lõrincz, PhD (IV/1707-6/2020/EKU)

Primary cells: Primary human peritoneal fibroblasts (phPFB), primary human colon fibroblasts (phCFB), primary human skin fibroblasts (phSFB), were prepared by enzymatic digestion of the related tissue using 1 mg/ml collagenase type II (Life Technologies Kft, Hungary), primary human peritoneal mesothelial cells (HPMC) were prepared by enzymatic digestion of the related tissue using 0.25% trypsin-EDTA (Life Technologies Kft, Hungary).

Primary fibroblasts (phFBs) were cultured at 37° C. in Dulbecco's modified Eagle's medium/Nutrient Mixture F-12 (DMEM-F12, Life Technologies Kft, Hungary) medium supplemented with 10% heat-inactivated foetal calf serum (FCS, Life Technologies Kft, Hungary), 100 μg/ml streptomycin and 100 U/ml penicillin (Life Technologies Kft, Hungary) at 37° C. in a humidified atmosphere of 5% CO₂ in air.

Primary HPMCs were cultured in M199 medium supplemented with 10% FCS, 400 nM hydrocortisone (Life Technologies Kft, Hungary), 870 nM insulin (Life Technologies Kft, Hungary), 20 mM HEPES (Life Technologies Kft, Hungary), 3.3 nM epithelial growth factor (EGF, R&D, UK), and 100 μg/ml streptomycin and 100 U/ml penicillin. Monolayers were identified as phFBs or HPMCs by their morphology and α-SMA (phFBs) or cytokeratin 18 positivity (HPMCs) by immunofluorescent staining.

HUVEC (#CRL-1730) human umbilical vein endothelial cells (ATCC, USA) were cultured in Vascular Cell Basal Medium (ATCC, USA), supplemented with Endothelial Cell Growth Kit-VEGF (ATCC, USA), 10% FCS, 100 μg/ml streptomycin and 100 U/mL penicillin at 37° C. in a humidified atmosphere of 5% CO₂ in air.

Peripheral blood mononuclear cells (PBMC) from control patient were isolated by density gradient centrifugation using Histopaque-1077 (Sigma-Aldrich, Hungary). After isolation, cells were placed into RPMI 1640 medium (ATCC, USA) supplemented with L-glutamine (Life Technologies Kft, Hungary), 10% FCS, 100 μg/ml streptomycin and 100 U/ml penicillin at 37° C. in a humidified atmosphere of 5% CO₂ in air.

Cell lines: MRC-5 (#CCL-171) human lung fibroblast, A549 (#CRM-CCL-185) human lung epithelial like adeno carcinoma cells, and Caco-2 (#HTB-37) human colon carcinoma cells (American Type Culture Collection (ATCC), USA) were cultured in Dulbecco's Modified Eagle Medium (Thermo Fisher Scientific, USA), HEPG2 human hepatocellular carcinoma cell line (American Type Culture Collection (ATCC), USA), were grown in in Dulbecco's modified Eagle's medium/Nutrient Mixture F-12 (DMEM-F12) medium. Hs578-T human breast carcinoma cell line (American Type Culture Collection (ATCC), USA) and LCLC-103H human large cell lung carcinoma cell line (German Collection of Microorganisms and Cell Cultures GmbH, Germany) were grown in RPMI 1640 medium (ATCC, USA). HT-29 (#HTB-38), human colon carcinoma cell line (ATCC) was cultured in McCoy's 5A Medium (Thermo Fisher Scientific, USA) supplemented with 10% FCS, 100 μg/ml streptomycin and 100 U/ml penicillin at 37° C. in a humidified atmosphere of 5% CO₂ in air.

Immunofluorescent staining: to characterize primary human cells isolated from tissue samples, immunofluorescent staining was performed. Monolayers were identified as phFBs or HPMCs by their morphology and α-SMA (phFBs) or CK18 immunopositivity (HPMCs). 6×10⁴ phPFB, phCFB, phSFB, and HPMC cells were seeded in 4-well cell culture slide (Corning Costar, Sigma-Aldrich, Hungary) at 80% confluence. After fixation step by methanol, chambers were incubated firstly with primary antibodies (1:500, Santa Cruz, USA) against the fibroblast marker smooth muscle actin alpha (α-SMA, Sigma-Aldrich, Hungary) and mesothelial marker cytokeratin 18 (CK18, Santa Cruz Biotechnology, Hungary) for 2 hours at room temperature. After multiple washing with PBS slides were incubated with the corresponding Alexa Fluor®-conjugated secondary antibodies (1:1000, Life Technologies Kft, Hungary) for 1 hour at room temperature. Appropriate controls were performed by omitting the primary antibodies to avoid autofluorescence. Nuclei were stained with Hoechst 33342 (1:1000, Sigma-Aldrich, Hungary) for 10 minutes at room temperature. Finally, slides were cover-slipped with ProLong™ Gold antifade mountant (Invitrogen, Hungary). Cell morphology, α-SMA, and CK18 immunopositivity was visualised using Olympus IX-81 fluorescent microscope system (Olympus Corporation, Japan).

EVs uptake experiments in vitro and in vivo: for testing the in vitro internalization of EVs, samples were labelled by using the liphophilic fluorescence dye, DiI (DiIC18 (3), 1,1′-dioctadecyl-3,3,3′,3′-tetramethylindocarbocyanine perchlorate, Molecular Probes, UK) following the manufacturer's instructions. Briefly, 100 μl EVs were incubated with 2 μl of 1 mg/ml DiI dye for 30 minutes at 37° C. Unbound dye was eliminated by SEC after washing the labelled EVs with 400 μl PBS. In parallel same volume of the dye was incubated in PBS (in the absence of EVs) and subjected to same incubation periods, then after wash step SEC was carried out as previously described. This latter sample was used as negative control.

6×10⁴ phPFB, phCFB, phSFB, and HPMC cells were seeded in 4-well cell culture slide (Corning Costar, Sigma-Aldrich, Hungary) at 80% confluence. Cells were then incubated with cell culture medium containing 10\hat{ }9 particles/ml of the DiI-labelled EVs for 24 hours at 37° C. in a humidified atmosphere of 5% CO₂. Subsequently, nuclei were stained with Hoechst 33342 (1:1000, Merck Kft, Hungary) for 10 minutes at room temperature. Finally, slides were cover-slipped with ProLong™ Gold antifade mountant (Invitrogen, Hungary). Internalized EVs were visualised using Olympus IX-81 fluorescent microscope system (Olympus Corporation, Japan). To test in vivo internalization of EVs of Chlorella sp. MACC-360 into the liver and skin, as well as MACC-1, MACC-3 and MACC-1023 into the visceral peritoneum and peritoneal lavage cells 1049 particles of the DiI labelled EVs was intravenously administered to the tail vain of C57/BL6 J mice or was intraperitoneally or intracutaneously injected. After 24 hours mice were sacrificed, thereafter liver, visceral peritoneum samples, peritoneal lavage cells and skin samples were harvested, 3 μm-thick sections were prepared—in case of liver and peritoneal samples- and finally slides were cover-slipped with ProLong™ Gold antifade mountant with DAPI (Invitrogen, Hungary). Internalized EVs were visualised using Olympus IX-81 fluorescent microscope system.

Western blot: PDE samples were lyophilised, dissolved in water-reaching equal protein concentrations-, then denatured by adding standard Leammli buffer and boiling for 5 minutes. Samples were separated by electrophoreses on 4-20% gradient SDS polyacrylamide gel (BioRad, USA), and transferred into nitrocellulose membranes (BioRad, USA). To verify the transfer, membranes were stained with Ponceau S (Sigma-Aldrich, Hungary), then washed and blocked with 5% non-fat milk in TRIS-buffered saline (TBS) for 1 hour at room temperature. Thereafter, membranes were incubated overnight at 4° C. with specific primary antibodies (1:1000) against the profibrotic factors (PDGF-BB, TGF-1/2/3 and CTGF, Santa Cruz, USA). After repeated washing with TBS containing 0.05% Tween-20 and 1% non-fat milk, membranes were incubated with the corresponding HRP-conjugated secondary antibodies (1:2000, Merck Kft, Hungary, Life Technologies Kft, Hungary) for 1 hour at room temperature. Immunoreactive bands of interest were detected using enhanced chemiluminescence detection (Western Blotting Luminol Reagent, GE Healthcare, USA).

MTT (cell proliferation and viability) assay: MTT assay was performed on platelet derived growth factor B (PDGF-B, 10 ng/ml, R&D Systems, USA) or peritoneal dialysate (PDE, 30%) treated phPFB, phCFB, phSFB, MRC-5, Caco2, HEPG2, Hs578-T, LCLC-103H, HT-29 and A549 cells, and on methylglyoxal (MGO, 300-700 μM, Merck Kft, Hungary) treated HPMC HUVEC, and phSFB/A cells as well as 5 mM hydroxymethylfurfural (HMF) treated phSFB/A cells in the presence or absence of 10\hat{ }7 or 10\hat{ }8 particles/ml EV. Vehicle treated cells (4 mM HCL in case of PDGF, 30% Fresenius 1.5% glucose-containing peritoneal dialysis fluid (PDF) in case of PDE, equal volume of PBS in case of MGO and EV) served as controls. 24 hours after treatments, cell proliferation/viability was determined by a colorimetric method, based on the intracellular mitochondrial dehydrogenase activity of the attached cells. Briefly, 10 μl of MTT reagent, containing 5 mg/ml thiazolyl blue tetrazolium bromide (diluted in sterile H₂O) was added into each well including cells and 100 μl of supernatant as well, then incubated at 37° C. for 4 hours. Thereafter, the supernatants were removed from cells using a pipette, and the intracellular MTT crystals were dissolved by adding 100 μl 1:1 mixture of DMSO and ethanol (all reagents were purchased from Merck, Germany). Absorbance was recorded at 570 nm and at 690 nm as background in a SPECTROstar Nano microplate reader using SPECTROstar Nano MARS v3.32 software (BMG Labtech, Germany). Results were normalized and determined as percentage ratio of control group values.

LDH (cytotoxicity) assay: LDH assay was performed on peritoneal dialysate (PDE, 30%) treated Hs578-T, LCLC-103H, HepG2, phCFB/A, and MGO (100-700 μM) treated HPMC, HUVEC and phCFB/A cells and HMF (10 mM) (Merck Kft, Hungary) or 3,4-Dideoxyglucosone-3-ene (DGE) (75 μM) (Biosynth Carbosynth, UK) treated phCFB/A cells in the presence or absence of 10\hat{ }7 or 10\hat{ }8 particles/ml EV. Vehicle treated cells (equal volume of PBS) served as controls. The extent of cell death was determined 24 hours after treatments by a colorimetric method, based on the lactate dehydrogenase (LDH) enzyme activity in the supernatant, released from damaged cells. Equal volumes (40 μl) of aspired media were mixed in a sterile 96-well plate with LDH reagent, containing 109 mM lactate, 3.3 mM β-nicotinamide-adenine-dinucleotide-hydrate, 2.2 U/ml diaphorase, 3 mM TRIS, 30 mM HEPES, 10 mM NaCl, 350 μM thiazolyl blue tetrazolium bromide (all reagents were purchased from Merck Kft, Hungary), then incubated at 37° C. for 1 hour. Absorbance was recorded at 570 nm and at 690 nm as background in a SPECTROstar Nano microplate reader using SPECTROstar Nano MARS v3.32 software (BMG Labtech, Germany). Results were normalized and determined as percentage ratio of control group values.

Sirius Red (collagen detection) assay: Sirius Red assay was performed on transforming growth factor beta (TGF-β, 1 nM, R&D Systems, USA) or PDE (30%) treated phPFB, phCFB, phSFB and MRC-5 lung cells in the presence or absence of 10\hat{ }7 or 10\hat{ }8 particles/ml EV. Vehicle treated (4 mM HCL in case of TGF-B, 30% PDF in case of PDE, PBS in case of EV) cells served as controls. 48 hours after treatments, collagen deposition was determined based on a basic histological dye SiriusRed, incorporating into the triple helical collagen molecules. After removing supernatants, cells were incubated in a fixative solution containing 26% EtOH, 3.7% formaldehyde, 2% glacial acetic acid for 15 minutes at room temperature. Samples were stained for 1 h at room temperature with 0.1% solution of SiriusRed (DirectRed80) dissolved in 1% acetic acid, then washed three times with 200 μl of 0.1 M HCl, and finally the bounded dye was dissolved by adding 100 μl of 0.1 M NaOH (all reagents were purchased from Merck, Germany). Absorbance was recorded at 544 nm and at 690 nm as background in a SPECTROstar Nano microplate reader using SPECTROstar Nano MARS v3.32 software (BMG Labtech, Germany). Results were normalized and determined as percentage ratio of control group values.

TAS (cell migration) assay: TAS assay was performed on epidermal growth factor (EGF, 10 ng/ml, R&D Systems, USA) treated phCFB, phSFB, and EGF or PDE (30%) treated Caco-2 cells in the presence or absence of 10\hat{ }8 particles/ml EV, according to Apor Veres-Székely et al. Vehicle treated (PBS in case of EGF and EV, 30% PDF in case of PDE) cells served as controls. Briefly, cells were seeded at near-full density into 96 well-plates, containing non-toxic gel barriers to create cell-free zones. After 24 hours of incubation, barriers were removed and wells were washed with PBS, thereafter cells were treated. Bright-field images of each well were taken using Olympus IX-81 microscope system (Olympus, Japan) at 0, 24, 48 and 72 hours after treatment. Cell-free gap area was measured using ImageJ 1.48v software and determined as a ratio of initial gap area at 0 hour.

Real-time reverse transcriptase polymerase chain reaction (qRT-PCR): Total RNA was isolated from EV-treated (10\hat{ }8 particles/ml for 24 hours) or from 1 μg/ml phytohemagglutinin (PHA) (Merck Kft, Hungary) and EV cotreated PBMCs by Geneaid Total RNA Mini Kit (Geneaid Biotech, Taiwan). Equal RNA was reverse-transcribed using Maxima First Strand cDNA Synthesis Kit for RT-qPCR (Thermo Fisher Scientific, USA) to generate first-stranded cDNA. The mRNA expression of inflammation or fibrosis related markers (IL10, IL13, PDGFB, TGFB, IL6, MCP1) was determined by real-time PCR using LightCycler 480 SYBR Green I Master enzyme mix on a LightCycler 96 system (Roche Diagnostics, USA). Results were analyzed using LightCycler 96 software v1.1.0.1320 (Roche Diagnostics). Relative mRNA expression was determined by comparison with ribosomal protein lateral stalk subunit PO (RPRPO) or 18s ribosomal RNA (RN18S) as internal control using the AACt method. Data were normalized and presented as the ratio of their control group values.

IL-6 protein level (ELISA): 104 A549 and phSFB/A cells were seeded into a 96 well plate and were treated with 100 ng/ml lipopolysaccharide (LPS, Merck Kft, Hungary), 1 μg/mlPHA, Merck Kft, Hungary), 10 μg/ml polyinosilic:polycytidilic acid [poly (I:C), Merck Kft, Hungary] in the presence or absence of 10\hat{ }8 particles/ml EVs of Chlorella sp. MACC-360 origin for 24 hours. Vehicle treated cells (PBS) served as controls. Protein level of IL-6 was measured by human IL-6 DuoSet ELISA (#DY206, R&D Systems, Inc, USA) according to the manufacturer's instructions.

Kinase array: 5×10⁵ phCFB/A cells were seeded into a 6 well plate and were treated with peritoneal dialysate (PDE, 30%) or recombinant mix (PDGF-B 10 ng/ml, TGF-β 0.5 nM, EGF 10 ng/ml, CTGF 100 ng/ml, R&D Systems, Inc, USA) in the presence or absence of 10\hat{ }8 particles/ml EVs of Chlorella sp. MACC-360 origin for 30 minutes. Vehicle treated cells [Fresenius 1.5% glucose-containing peritoneal dialysis fluid (PDF), PBS] served as controls. Proteom profiler human phospho-kinase array kit (#ARY003C, R&D Systems, Inc, USA) was used following the manufacturer's instruction to determinate of the relative levels of human protein kinase phosphorylation.

Testing the EV stability: Ultrafiltrated and concentrated samples—by TFF-easy filters—of Chlorella sp. MACC-360 origin was incubated with (1) hydrochloric acid (HCl, Merck Kft, Hungary) for 1 hour at pH 4,6 at room temperature, (2) was treated with 0.25% trypsin-EDTA (TFF: trypsin-EDTA=2:1 ratio) for 1 hour at room temperature, (3) was incubated with HCl at pH 2-3 at room temperature-, (4) or 95° C. for 1 hour, than neutralized with sodium hydroxide (Merck Kft, Hungary), (5) was incubated at 95° C. for 1 hour and (6) was incubated with 20 μg/ml proteinase K (Life Technologies Kft, Hungary) for 1 hour at 37° C. After the above mentioned treatments SEC was carried out in case of all groups to isolate homogenous EV fractions. To test the effectivity of the resulted

EVs MTT assay was performed on platelet derived growth factor B (PDGF-B, 10 ng/ml) or peritoneal dialysate (PDE, 30%) treated phCFB/A cells according to the protocol as we described above.

Testing the effect of EV-EV and EV-platelet derived growth factor receptor alpha and beta (PDGFR-α, PDGFR-β) inhibitor combinations on the proliferation: Effect of EV of Chlorella sp. MACC-360 origin in combination with MACC-1 and MACC-908 as well as PDGFR-α inhibitor Ponatinib (Merck Kft, Hungary) and PDGFR-β inhibitor Axitinib (Merck Kft, Hungary) on peritoneal dialysis effluent (PDE) induced proliferation was determined on primary human colon fibroblast from patient A (phCFB/A). Briefly, phCFB/A cells were treated with PDE and 3×10\hat{ }8 particles/ml EVs of MACC-360, MACC-1 and MACC-908 origin and their combination of 1 μM Ponatinib or Axitinib in the presence or absence of 3×10\hat{ }8 particles/ml EV of Chlorella sp. MACC-360 origin for 24 hours. Proliferation was determined by MTT assay according to the above mentioned protocol (see MTT block).

Effect of in vivo EV treatment in mice model of peritoneal fibrosis and DSS induced colitis: Animal experiments were performed on 6-8-week-old male C57Bl/6J mice (Charles River Labratories, Germany). Animals were housed in a temperature-controlled (22±1° C.) room with alternating light and dark cycles and had free access to standard laboratory rodent chow and water. The institutional committee on animal welfare approved all experiments (PEI/001/1731-9/2015). Mice were randomized into four groups:
(A) vehicle-treated (PBS) mice served as controls (n=6),
(B) mice were intraperitoneal (i.p.) injected at 2nd and 5th days with 300 μl of 10\hat{ }9 particles/ml EV in PBS (n=6).
(C) mice were i.p. injected daily with 300 μl of 0.1% chlorhexidine digluconate (CG) with 15% ethanol in PBS (n=6);
(D) mice received the same EV and CG treatments together (4 hours apart) as group B and C (n=8);

In case of the DSS induced colitis model C57BL/6J mice received 2.5% DSS in their drinking water for 7 days. During the DSS treatment mice were treated by daily oral gavage of 100 μl of about 1.5 10\hat{ }10 particles/ml EV of Chlorella sp. MACC-360 or vehicle (PBS) only (DSS or DSS+MACC-360 groups (n=8), respectively). Changes in the disease activity index (DAI) were monitored during the whole experiment. At the end of treatment (after 7 days) mice were sacrificed, parietal peritoneal samples were harvested and fixed in 4% buffered formaldehyde or snap-frozen and stored at −80° C. until further molecular biological measurements.

Masson's Trichrome staining: Formalin-fixed peritoneum were embedded in paraffin and prepared in 3 μm-thick sections. For evaluation the extent of peritoneal fibrosis, standard Masson's Trichrome histological staining was performed, then tissue sections were photographed using Olympus IX-81 microscope system (Olympus, Japan). The collagen-rich tissue area (blue colour) was quantitatively measured using ImageJ 1.48v software. The 5 thickness of the sub-mesothelial tissue was determined as the average of 10 independent measurements of non-overlapping areas in case of each section.

Statistical Analysis: Data were analyzed using GraphPad Prism 8.0. software (GraphPad Software Inc., USA). After testing normality by Kolmogorov-Smirnov test, analysis of significance was performed by unpaired t-test or Mann-Whitney test. In case of the EV-EV and EV-combination testing two-way ANOVA was used to analyse 10 the differences. Results were illustrated as mean±SD of the corresponding groups. The applied tests, significances, and number of elements (n) are indicated in each figure legend.

1. A composition consisting essentially of algal extracellular vesicles (EVs) as active agents and one or more additional substance useful as carrier,
for use in therapy of a disorder selected from the group consisting of tumours and fibroproliferative diseases, said disorder being associated with cell proliferation, cell migration and dysregulated extracellular matrix (ECM) formation,
wherein the EVs are for use in inhibiting cell migration and/or proliferation and/or the EVs are for use in inhibiting ECM production and/or deposition.

2. The composition for use in therapy according to claim 1 for use in the therapy of a fibroproliferative diseases by the inhibition of ECM production.

3. The composition for use in therapy according to claim 1 for use in the therapy of a tumour by the inhibition of cell migration and proliferation.

4. The composition for use in therapy according to claim 1 for use in the therapy of a fibroproliferative diseases by the inhibition of cell proliferation and migration.

5. The composition for use in therapy according to claim 1 for use in the therapy of a tumour by the inhibition of ECM production and/or deposition.

6. The composition for use in therapy according to claim 1, wherein the EVs are for use in inhibiting
EGF, PDGF-BB and/or TGFβ induced cell proliferation,
EGF, PDGF-BB and/or TGFβ induced cell migration, and/or
EGF, PDGF-BB and/or TGFβ induced ECM production/deposition.

7. The composition for use in therapy according to claim 1 wherein said EVs are for use in inhibiting the activity of a signaling pathway selected from the group consisting of
PDGF-BB induced signaling pathway,
TGFβ induced signaling pathways,
EGF induced signaling pathway and/or
a signalling pathway induced by a mixture of growth factors, in particular a comprising PDGF-BB, TGFβ and EGF.

8. The composition for use in therapy according to claim 1 wherein said EVs are for use in inhibiting the activity of
a signalling pathway induced by peritoneal dialysis effluent (PDE).

9. The composition for use in therapy according to claim 1 wherein
the EVs are for use in inhibiting cell migration, preferably EGF or PDE induced cell migration, and/or
the EVs are for use in inhibiting cell proliferation, preferably PDGF-BB or PDE induced cell proliferation.

10. The composition for use in therapy according to claim 1 wherein the EVs are for use in regulating ECM production/deposition, preferably inhibiting ECM production/deposition, preferably TGFβ or PDE induced ECM production.

11. The composition for use in therapy according to claim 1 wherein the EVs are green algae.

12. The composition for use in therapy according to claim 1 wherein the EVs are derived from prokaryotic photoautotroph algae.

13. The composition for use in therapy according to claim 1 wherein the EVs are derived from eukaryotic green algae.

14. The composition for use in therapy according to claim 11 wherein the EVs are derived
from the eukaryotic green algae Chlorophyta or Streptophyta phylum.

15. The composition for use in therapy according to claim 12 wherein the EVs are derived from freshwater algae, preferably from freshwater green algae and/or eukaryotic freshwater algae.

16. The composition for use in therapy according to claim 1 wherein the EVs are derived
from the Rhodophyta or Ochrophytaphylum.

17. The composition for use in therapy according to claim 1 wherein the EVs are derived
from the Cyanobacteriota phylum.

18. The composition for use in therapy according to claim 1, wherein the EVs are derived from Chlorella sorokiniana, Parachlorella kessleri, Chlorella vulgaris, Chlorella pyrenoidosa (Auxenochlorella pyrenoidosa), Chlamydomonas reinhardtii, Hormidiospora verrucosa, Haematococcus lacustris, Tetraselmis chui, Zygnema peliosporum, Klebsormidium nitens, Spirogyra sp., Vischeria polyphem, Ascophyllum nodosum, Palmaria palmata, Microcystis aeruginosa, Nostoc linckia, Spirulina platensis (Arthrospira platensis), Spirulina maxima (Limnospira maxima), Arthronema africanum, Synechococcus sp., Aphanizomenon flos-aquae.

19. The composition for use in therapy according to claim 1, wherein the disorder is a disorder selected from a gastrointestinal disorder, a disorder of the skin, a disorder of the lung, a disorder of the kidney, a disorder of the liver and cardiovascular disorder and a disorder of the peritoneum or the disorder is in the brain or in the eye or diseases of the urogenital tract.

20. The composition for use in therapy according to claim 19, wherein the disorder is a fibroproliferative disorder of any one of said tissues, characterized by disregulated ECM production/deposition in said tissue.

21. The composition for use in therapy according to claim 19, wherein the disorder is a neoplastic disorder of any one of said tissues.

22. The composition for use in therapy according to claim 1, said composition consisting essentially of algal extracellular vesicles (EVs) as active agent for use in method for treatment of a subject as defined in any of claims 1 to 15, and biologically acceptable carrier for a mammalian subject.

23. The composition for use in therapy according to claim 1, said composition being a pharmaceutical composition comprising algal EVs as an active agent and a pharmaceutically acceptable excipient.

24. The composition for use in therapy according to claim 1 said composition being a nutraceutical composition comprising algal EVs as an active agent and a nutraceutically acceptable excipient.

25. A composition according to claim 1 comprising algal extracellular vesicles (EVs) as active agent for use as active agent in method for treatment of a subject, wherein said composition comprising a combination of EVs from multiple algae.
